(19) United States
(12) Patent Application Publication
(10) Pub. No.: US 2023/0089255 A1
(43) Pub. Date: Mar. 23, 2023

(54) COMBINATIONS OF DGK INHIBITORS AND CHECKPOINT ANTAGONISTS

(71) Applicant: BRISTOL-MYERS SQUIBB COMPANY, Princeton, NJ (US)

(72) Inventors: Susan WEE, Moorestown, NJ (US); Joseph L. BENC, Princeton, NJ (US); Xinyu WANG, Pennington, NJ (US); Upender VELAPARTHI, Princeton Junction, NJ (US); Louis S. CHUPAK, Old Saybrook, CT (US); Chatan P. DARNE, Ewing, NJ (US); Min DING, Stow, MA (US); Robert G. GENTLES, Killingworth, CT (US); Yazhong HUANG, Wallingford, CT (US); Scott W. MARTIN, Middletown, CT (US); Ivar M. MCDONALD, Woodstock, CT (US); Richard E. OLSON, Cambridge, MA (US); Xiaofan ZHENG, Cheshire, CT (US); John S. TOKARSKI, Princeton, NJ (US); Bireshwar DASGUPTA, Doylestown, PA (US); Manjunatha Narayana Rao KAMBLE, Bangalore (IN); Raju MANNOORI, Bangalore (IN); Haslbur RAHAMAN, Bangalore (IN); Prasada Rao JALAGAM, Bangalore (IN); Saumya ROY, Bangalore (IN); Gopikishan TONUKUNURU, Bommasandra Bangalore Karnataka (IN); Sivasudar VELAIAH, Hosur Tamilnadu (IN); Jayakumar Sankara WARRIER, Bangalore (IN); Kotha Rathnakar REDDY, Warangal West Talangana State (IN); Thiruvenkadam RAJA, Tamil Nadu (IN); Denise GRUNENFELDER, Boston, MA (US); Michael WICHROSKI, Newbuy, MA (US)

(21) Appl. No.: 17/786,442

(22) PCT Filed: Dec. 18, 2020
(86) PCT No.: PCT/US2020/066198

§ 371 Date: Jun. 16, 2022

Related U.S. Application Data
(60) Provisional application No. 62/950,570, filed on Dec. 19, 2019.

(51) Int. Cl. A61K 31/519; A61K 31/496; A61K 39/395; A61P 35/00

(52) U.S. Cl. CPC A61K 31/519; A61K 31/496; A61K 39/3955; A61P 35/00

(57) ABSTRACT

Provided are inhibitors of diacylglycerol kinases (DGK) and methods for treating diseases that would benefit from the stimulation of the immune system, such as cancer and infections diseases, comprising administering a DGK inhibitor in combination with an antagonist of the PD 1/PD-L 1 axis and/or an antagonist of CTLA4.

### CROSS REFERENCE

This application claims the benefit of U.S. Provisional Application No. 62/950,570 filed Dec. 19, 2019, which is incorporated herein in its entirety.

### BACKGROUND

Human cancers harbor numerous genetic and epigenetic alterations, generating neoantigens potentially recognizable by the immune system (Sjoblom et al. (2006) Science 314:268-74). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system would make immunotherapy unique among all cancer treatment modalities. However, although an endogenous immune response to cancer is observed in preclinical models and patients, this response is ineffective, and established cancers are viewed as “self” and tolerated by the immune system. Contributing to this state of tolerance, tumors may exploit several distinct mechanisms to actively subvert anti-tumor immunity. These mechanisms include dysfunctional T-cell signaling (Mizoguchi et al., (1992) Science 258:1795-98), suppressive regulatory cells (Facciabene et al., (2012) Cancer Res. 72:2162-71), and the co-opting of endogenous “immune checkpoints”, which serve to down-modulate the intensity of adaptive immune responses and protect normal tissues from collateral damage, by tumors to evade immune destruction (Topalian et al., (2012) Curr. Opin. Immunol. 24:1-6; Mellman et al. (2011) Nature 480:480-489).

Diacylglycerol kinases (DGKs) are lipid kinases that mediate the conversion of diacylglycerol to phosphatidic acid thereby terminating T cell functions propagated through the TCR signaling pathway. Thus, DGKs serve as intracellular checkpoints and inhibition of DGKs are expected to enhance T cell signaling pathways and T cell activation. Supporting evidence include knock-out mouse models of either DGKa or DGKζ which show a hyper-responsive T cell phenotype and improved anti-tumor immune activity (Riese M. J. et al., Journal of Biological Chemistry, (2011) 7: 5254-5265; Zha Y et al., Nature Immunology, (2006) 12:1343; Olenchock B. A. et al., (2006) 11: 1174-81). Furthermore tumor infiltrating lymphocytes isolated from human renal cell carcinoma patients were observed to overexpress DGKa which resulted in inhibited T cell function (Prinz, P. U. et al., J Immunology (2012) 12:5990-6000). Thus, DGKa and DGKζ are viewed as targets for cancer immunotherapy (Riese M. J. et al., Front Cell Dev Biol. (2016) 4: 108; Chen, S. S. et al., Front Cell Dev Biol. (2016) 4: 130; Avila-Flores, A. et al., Immunology and Cell Biology (2017) 95: 549-563; Noessner, E., Front Cell Dev Biol. (2017) 5: 16; Krishna, S., et al., Front Immunology (2013) 4:178; Jing, W. et al., Cancer Research (2017) 77: 5676-5686.

### SUMMARY

Provided herein are methods of treating a disease or disorder comprising administering to a subject an inhibitor of DGKα, DGKζ, or both DGKα and DGKζ, such as a compound of Formula (I) or (II), such as a compound selected from compounds 1 to 34 or a pharmaceutically acceptable salt thereof in combination with an antagonist of the PD1/PD-L1 axis and/or an antagonist of CTLA4. Exemplary diseases or disorders include those that would benefit from the stimulation of the immune system, such as cancer and infectious diseases. Also provided are uses of an inhibitor of DGKα, DGKζ, or both DGKα and DGKζ, such as a compound of Formula (I) or (II), such as a compound selected from compounds 1 to 34 or a pharmaceutically acceptable salt thereof, for the manufacture of a medicament for the treatment of diseases or disorders, such as those that would benefit from the stimulation of the immune system, such as cancer and infectious diseases, and wherein the inhibitor is administered in combination with an antagonist of the PD1/PD-L1 axis and/or an antagonist of CTLA4. Provided herein are uses of an inhibitor of DGKα, DGKζ, or both DGKα and DGKζ, such as a compound of Formula (I) or (II), such as a compound selected from compounds 1 to 34 or a pharmaceutically acceptable salt thereof, for the manufacture of a medicament for the treatment of diseases or disorders, such as those that would benefit from the stimulation of the immune system, such as cancer and infectious diseases, and wherein the inhibitor is administered in combination with an antagonist of the PD1/PD-L1 axis and an antagonist of CTLA4.

Also provided are uses of an antagonist of the PD1/PD-L1 axis, for the manufacture of a medicament for the treatment of diseases or disorders, such as those that would benefit from the stimulation of the immune system, such as cancer and infectious diseases, and wherein the antagonist is administered in combination with an inhibitor of DGKα, DGKζ, or both DGKα and DGKζ, such as a compound of Formula (I) or (II), such as a compound selected from compounds 1 to 34, or a pharmaceutically acceptable salt thereof and/or an antagonist of CTLA4. Provided are uses of an antagonist of the PD1/PD-L1 axis, for the manufacture of a medicament for the treatment of diseases or disorders, such as those that would benefit from the stimulation of the immune system, such as cancer and infectious diseases, and wherein the antagonist is administered in combination with an inhibitor of DGKα, DGKζ, or both DGKα and DGKζ, such as a compound of Formula (I) or (II), such as a compound selected from compounds 1 to 34, or a pharmaceutically acceptable salt thereof and an antagonist of CTLA4.

Also provided are uses of an antagonist of CTLA4, for the manufacture of a medicament for the treatment of diseases or disorders, such as those that would benefit from the stimulation of the immune system, such as cancer and infectious diseases, and wherein the antagonist is administered in combination with an inhibitor of DGKα, DGKζ, or both DGKα and DGKζ, such as a compound of Formula (I) or (II), such as a compound selected from compounds 1 to 34, or a pharmaceutically acceptable salt thereof and/or an antagonist of the PD1/PD-L1 axis. Provided are uses of an antagonist of CTLA4, for the manufacture of a medicament for the treatment of diseases or disorders, such as those that would benefit from the stimulation of the immune system, such as cancer and infectious diseases, and wherein the antagonist is administered in combination with an inhibitor of DGKα, DGKζ, or both DGKα and DGKζ, such as a compound of Formula (I) or (II), such as a compound selected from compounds 1 to 34, or a pharmaceutically acceptable salt thereof and an antagonist of the PD1/PD-L1 axis.

Exemplary compounds, such as compounds of Formula I described herein and pharmaceutically acceptable salts thereof, are described in PCT/US2019/039131, filed Jun. 26, 2019, and PCT/US2019/039135, filed Jun. 26, 2019, the contents of both of which are specifically incorporated by reference herein. Exemplary compounds, such as compounds of Formula II described herein and pharmaceutically acceptable salts thereof, are described in PCT/US2020/048070, filed Aug. 27, 2020, the contents of which are specifically incorporated by reference herein.

These and other features of the new methods of treatments will be set forth in expanded form as the disclosure continues.

### BRIEF DESCRIPTION OF THE FIGURES

FIGS. 1 A and B show enhanced IFN-γ secretion from T cells incubated with increasing concentrations of DGKi and nivolumab (A) or ipilimumab (B) in an MLR assay relative to the same assays in the absence of nivolumab or ipilimumab.

FIGS. 2A-H show that the triple combination of DGKi with an anti-PD-1 antibody and an anti-CTLA4 antibody slows tumor growth relative to that in mice treated only with an anti-PD-1 antibody and an anti-CTLA4 antibody. FIGS. 2A-H show tumor size over time post-implant of mouse B16 melanoma cells to mice, and treated with vehicle alone (FIG. 2A), anti-PD-1 antibody alone (FIG. 2B), anti-PD-1 antibody and anti-CTLA4 antibody (FIG. 2C), DGKi and anti-PD-1 antibody (FIG. 2D), DGKi and anti-CTLA4 antibody (FIG. 2E), DGKi alone (FIG. 2F), DGKi and anti-PD-1 antibody and anti-CTLA4 antibody (FIG. 2G). FIG. 2H shows the average tumor size post-implant of the B16 cells in mice treated with (i) anti-PD-1 antibody and anti-CTLA4 antibody, (ii) DGKi and anti-PD1 antibody, (iii) DGKi and CTLA4 antibody and (iv) DGKi and anti-PD1 antibody and anti-CTLA4 antibody.

FIGS. 3 A-I show that combination treatments with an inhibitor of DGK and an anti-PD-1 antibody and/or an anti-CTLA4 antibody result in improved complete responses (FIG. 3A) and that the increased level of response correlates with increased AH1+CD8 T cells (FIG. 3B) in the CT26 mouse model.

FIGS. 4A-F show that inhibition of DGK lowers the antigen threshold required for TCR activation. FIGS. 4A-F show the levels of IL-2 secreted from OT1 CD8 T cells incubated with increasing levels of antigen and presenting one of the peptides OVA (A), A2 (B), Q4 (C), T4 (D) and Q4H7 (E), which shows that DGK inhibition will lower the concentration of tumor antigen required for T cell activation. FIG. 4F shows the level of IL-2 secreted at 1000 ng/ml of each of the peptides shown in FIGS. 4A-E, as well as that obtained with the scrambled peptide, which shows that DGK inhibition will potentiate the T cell response induced by weak tumor antigens.

FIGS. 5 A and B show that inhibition of DGK increases human CTL effector function and enhances tumor cell killing. FIG. 5A shows the level of IFN-γ secretion from T cells incubated with a peptide in the presence of increasing concentrations of DGKi. FIG. 5B shows increased tumor cell killing at day 3 upon incubation of the tumor cells with increased cognate peptide.

FIGS. 6 A and B, indicate that DGKi can overcome decreased B2M levels to restore T cell effector function. FIG. 6A shows the level of 02 microglobulin in CRISPR KO of B2M in HCT116 cells. FIG. 6 B shows that DGKi increases IFN-γ levels.

FIG. 7 shows the tumor volume as a function of days post implant of tumor cells in the CT26 animal model in mice treated with DGKi Compound 16 and an anti-PD-1 antibody in the presence or absence of a CD8 depleting antibody, showing that the presence of CD8 depleting antibody reduces tumor reduction.

FIG. 8 shows the tumor volume as a function of days post implant of tumor cells in the CT26 animal model in mice treated with DGKi Compound 16 and an anti-PD-1 antibody in the presence or absence of a CD4 depletion antibody, showing that the presence of CD4 depleting antibody stimulates tumor reduction.

FIG. 9 shows the tumor volume as a function of days post implant of tumor cells in the CT26 animal model in mice treated with DGKi Compound 16 and an anti-PD-1 antibody in the presence or absence of an NK cell depleting antibody, showing that the presence of NK cell depleting antibody reduces tumor reduction.

FIG. 10 shows that the combination of DGKi with either anti-PD-1 or anti-CTLA4 is capable of eliciting complete tumor regression (CR) in the MC38 tumor model. The tumor volume for individual animals is presented after treatment with only vehicle (FIG. 10A), DGKi (FIG. 10B), anti-PD-1 (FIG. 10C), anti-CTLA4 (FIG. 10D), DGKi and anti-PD-1 (FIG. 10E) or DGKi and anti-CTLA4 (FIG. 10F). DGKi, anti-PD-1 and anti-CTLA4 monotherapies are each capable of delaying tumor growth. The combination of DGKi and anti-PD-1 elicits CR of tumors in 100% of the animal tested while the combination of DGKi and anti-CTLA4 elicits CR in 70% of the mice tested.

FIG. 11 shows that the addition of DGKi to anti-PD-1 therapy can elicit complete regression (CR) of tumors in both the MC38 and CT26 animal models and that cured animals from these groups develop immunological memory sufficient to reject tumor re-challenge. The tumor volume for individual animals is presented after treatment with only vehicle (FIGS. 11A & 11E), anti-PD-1 (FIGS. 11B & 11F) or anti-PD-1 and DGKi (FIGS. 11C & 11G). DGKi elicits a robust combination effect with anti-PD-1 resulting in 100% and 60% CR of tumors in the MC38 and CT26 models, respectively. To assess immunological memory in cured animals, mice were re-challenged with 10× the number of tumor cells used for the initial implant and tumor volume was measured as a function of days post implant. All of the re-challenged animals in the MC38 cohort (FIG. 11D) and CT26 cohort (FIG. 11H) spontaneously rejected tumors confirming that DGKi and anti-PD-1 combination therapy elicits long-term immunological memory.

FIG. 12 shows that anti-PD-1, anti-CTLA4 and DGKi triple therapy can reduce tumor growth in the checkpoint inhibitor refractory B16F10 tumor model. The tumor volume for individual animals is presented after treatment with only vehicle (FIG. 12A), anti-PD-1 and anti-CTLA4 (FIG. 12B), anti-PD-1 and DGKi (FIG. 12C), anti-CTLA4 and DGKi (FIG. 12D) or anti-PD-1, anti-CTLA4 and DGKi (FIG. 12E). The mean tumor volumes for each group is presented in FIG. 12F.

### DETAILED DESCRIPTION

Provided herein are methods of treating a proliferative disease, such as cancer, or a viral infection, or more generally, a disease, disorder or condition that benefits from the stimulation of the immune system, as well as any disease, disorder or condition that can be prevented, ameliorated, or cured by inhibiting DGKα and/or DGKζ enzyme activity, comprising administering to a subject in need thereof, a therapeutically effective amount of an inhibitor of DGKα and/or DGKζ or a pharmaceutically acceptable salt thereof and (i) an antagonist of the PD1/PD-L1 axis (e.g., an antagonist of human PD1 or human PD-L1) and/or (ii) an antagonist of human CTLA4.

### Definitions

The features and advantages of the methods of treatment may be more readily understood by those of ordinary skill in the art upon reading the following detailed description. It is to be appreciated that certain features of the methods of treatment that are, for clarity reasons, described above and below in the context of separate embodiments, may also be combined to form a single embodiment. Conversely, various features of the methods of treatment that are, for brevity reasons, described in the context of a single embodiment, may also be combined so as to form sub-combinations thereof. Embodiments identified herein as exemplary or preferred are intended to be illustrative and not limiting.

Unless specifically stated otherwise herein, references made in the singular may also include the plural. For example, “a” and “an” may refer to either one, or one or more.

As used herein, the phrase “compounds and/or pharmaceutically acceptable salts thereof” refers to at least one compound, at least one salt of the compounds, or a combination thereof. For example, compounds of Formula (I) and/or pharmaceutically acceptable salts thereof includes a compound of Formula (I); two compounds of Formula (I); a pharmaceutically acceptable salt of a compound of Formula (I); a compound of Formula (I) and one or more pharmaceutically acceptable salts of the compound of Formula (I); and two or more pharmaceutically acceptable salts of a compound of Formula (I).

Unless otherwise indicated, any atom with unsatisfied valences is assumed to have hydrogen atoms sufficient to satisfy the valences.

The definitions set forth herein take precedence over definitions set forth in any patent, patent application, and/or patent application publication incorporated herein by reference.

Listed below are definitions of various terms used herein. These definitions apply to the terms as they are used throughout the specification (unless they are otherwise limited in specific instances) either individually or as part of a larger group.

Throughout the specification, groups and substituents thereof may be chosen by one skilled in the field to provide stable moieties and compounds.

In accordance with a convention used in the art,

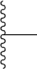

is used in structural formulas herein to depict the bond that is the point of attachment of the moiety or substituent to the core or backbone structure.

The terms “halo” and “halogen,” as used herein, refer to F, Cl, Br, and I.

The term “cyano” refers to the group —CN.

The term “amino” refers to the group —NH₂.

The term “oxo” refers to the group ═O.

The term “alkyl” as used herein, refers to both branched and straight-chain saturated aliphatic hydrocarbon groups containing, for example, from 1 to 12 carbon atoms, from 1 to 6 carbon atoms, and from 1 to 4 carbon atoms. Examples of alkyl groups include, but are not limited to, methyl (Me), ethyl (Et), propyl (e.g., n-propyl and i-propyl), butyl (e.g., n-butyl, i-butyl, sec-butyl, and t-butyl), and pentyl (e.g., n-pentyl, isopentyl, neopentyl), n-hexyl, 2-methylpentyl, 2-ethylbutyl, 3-methylpentyl, and 4-methylpentyl. When numbers appear in a subscript after the symbol “C”, the subscript defines with more specificity the number of carbon atoms that a particular group may contain. For example, “C₁₋₄ alkyl” denotes straight and branched chain alkyl groups with one to four carbon atoms.

The term “fluoroalkyl” as used herein is intended to include both branched and straight-chain saturated aliphatic hydrocarbon groups substituted with one or more fluorine atoms. For example, “C₁₋₄ fluoroalkyl” is intended to include C₁, C₂, C₃, and C₄ alkyl groups substituted with one or more fluorine atoms. Representative examples of fluoroalkyl groups include, but are not limited to, —CF₃ and —CH₂CF₃.

The term “cyanoalkyl” includes both branched and straight-chain saturated alkyl groups substituted with one or more cyano groups. For example, “cyanoalkyl” includes —CH₂CN, —CH₂CH₂CN, and C₁₋₄ cyanoalkyl.

The term “aminoalkyl” includes both branched and straight-chain saturated alkyl groups substituted with one or more amine groups. For example, “aminoalkyl” includes —CH₂NH₂, —CH₂CH₂NH₂, and C₁₋₄ aminoalkyl.

The term “hydroxyalkyl” includes both branched and straight-chain saturated alkyl groups substituted with one or more hydroxyl groups. For example, “hydroxyalkyl” includes —CH₂OH, —CH₂CH₂OH, and C₁₋₄ hydroxyalkyl.

The term “alkenyl” refers to a straight or branched chain hydrocarbon radical containing from 2 to 12 carbon atoms and at least one carbon-carbon double bond. Exemplary such groups include ethenyl or allyl. For example, “C₂₋₆ alkenyl” denotes straight and branched chain alkenyl groups with two to six carbon atoms.

The term “alkynyl” refers to a straight or branched chain hydrocarbon radical containing from 2 to 12 carbon atoms and at least one carbon to carbon triple bond. Exemplary such groups include ethynyl. For example, “C₂₋₆ alkynyl” denotes straight and branched chain alkynyl groups with two to six carbon atoms.

The term “cycloalkyl,” as used herein, refers to a group derived from a non-aromatic monocyclic or polycyclic hydrocarbon molecule by removal of one hydrogen atom from a saturated ring carbon atom. Representative examples of cycloalkyl groups include, but are not limited to, cyclopropyl, cyclopentyl, and cyclohexyl. When numbers appear in a subscript after the symbol “C”, the subscript defines with more specificity the number of carbon atoms that a particular cycloalkyl group may contain. For example, “C₃₋₆ cycloalkyl” denotes cycloalkyl groups with three to six carbon atoms.

The term “alkoxy,” as used herein, refers to an alkyl group attached to the parent molecular moiety through an oxygen atom, for example, methoxy group (—OCH₃). For example, “C₁₋₃ alkoxy” denotes alkoxy groups with one to three carbon atoms.

The terms “fluoroalkoxy” and “—O(fluoroalkyl)” represent a fluoroalkyl group as defined above attached through an oxygen linkage (—O—). For example, “C₁₋₄ fluoroalkoxy” is intended to include C₁, C₂, C₃, and C₄ fluoroalkoxy groups.

The term “alkalenyl” refers to a saturated carbon chain with two attachment points to the core or backbone structure. The alkalenyl group has the structure —(CH₂)ₙ— in which n is an integer of 1 or greater. Examples of alkalenyl linkages include —CH₂CH₂—, —CH₂CH₂CH₂—, and —(CH₂)₂₋₄—.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Compound, e.g., the compounds of Formula (I), can form pharmaceutically acceptable salts which can be used in the methods described herein. Unless otherwise indicated, reference to a compound is understood to include reference to one or more pharmaceutically acceptable salts thereof. The term “salt(s)” denotes acidic and/or basic pharmaceutically acceptable salts formed with inorganic and/or organic acids and bases. In addition, the term “salt(s) may include zwitterions (inner salts), e.g., when a compound of Formula (I) contains both a basic moiety, such as an amine or a pyridine or imidazole ring, and an acidic moiety, such as a carboxylic acid. Pharmaceutically acceptable (i.e., non-toxic, physiologically acceptable) salts are preferred, such as, for example, acceptable metal and amine salts in which the cation does not contribute significantly to the toxicity or biological activity of the salt. However, other salts may be useful, e.g., in isolation or purification steps which may be employed during preparation, and thus, are contemplated herein. Salts of compounds, e.g., the compounds of the formula (I), may be formed, for example, by reacting a compound, e.g., a compound of the Formula (I), with an amount of acid or base, such as an equivalent amount, in a medium such as one in which the salt precipitates or in an aqueous medium followed by lyophilization.

Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, maleates (formed with maleic acid), 2-hydroxyethanesulfonates, lactates, methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Exemplary basic salts include ammonium salts, alkali metal salts such as sodium, lithium, and potassium salts; alkaline earth metal salts such as calcium and magnesium salts; barium, zinc, and aluminum salts; salts with organic bases (for example, organic amines) such as trialkylamines such as triethylamine, procaine, dibenzylamine, N-benzyl-β-phenethylamine, 1-ephenamine, N,N′-dibenzylethylene-diamine, dehydroabietylamine, N-ethylpiperidine, benzylamine, dicyclohexylamine or similar pharmaceutically acceptable amines and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others. Preferred salts include monohydrochloride, hydrogensulfate, methanesulfonate, phosphate or nitrate salts.

Compounds, e.g., the compounds of Formula (I) can be provided as amorphous solids or crystalline solids. Lyophilization can be employed to provide the compounds, e.g., the compounds of Formula (I), as a solid.

It should further be understood that solvates (e.g., hydrates) of compounds, e.g., the compounds of Formula (I), can also be used in the methods described herein. The term “solvate” means a physical association of a compound, e.g., a compound of Formula (I), with one or more solvent molecules, whether organic or inorganic. This physical association includes hydrogen bonding. In certain instances the solvate will be capable of isolation, for example when one or more solvent molecules are incorporated in the crystal lattice of the crystalline solid. “Solvate” encompasses both solution-phase and isolable solvates. Exemplary solvates include hydrates, ethanolates, methanolates, isopropanolates, acetonitrile solvates, and ethyl acetate solvates. Methods of solvation are known in the art.

Various forms of prodrugs are well known in the art and are described in:
a) The Practice of Medicinal Chemistry, Camille G. Wermuth et al., Ch 31, (Academic Press, 1996);
b) Design of Prodrugs, edited by H. Bundgaard, (Elsevier, 1985);
c) A Textbook of Drug Design and Development, P. Krogsgaard-Larson and H. Bundgaard, eds. Ch 5, pgs 113-191 (Harwood Academic Publishers, 1991); and
d) Hydrolysis in Drug and Prodrug Metabolism, Bernard Testa and Joachim M. Mayer, (Wiley-VCH, 2003).

In addition, compounds, e.g., compounds of Formula (I), subsequent to their preparation, can be isolated and purified to obtain a composition containing an amount by weight equal to or greater than 99% of a compound, e.g., a compound of Formula (I), (“substantially pure”), which is then used or formulated as described herein. Such “substantially pure” compounds, e.g., compounds of Formula (I), are also contemplated herein.

“Stable compound” and “stable structure” are meant to indicate a compound that the compound is sufficiently robust to survive isolation to a useful degree of purity from a reaction mixture, and formulation into an efficacious therapeutic agent. The compounds for use herein are intended to embody stable compounds.

The compounds described herein are intended to include all isotopes of atoms occurring in the present compounds. Isotopes include those atoms having the same atomic number but different mass numbers. By way of general example and without limitation, isotopes of hydrogen include deuterium (D) and tritium (T). Isotopes of carbon include ¹³C and ¹⁴C. Isotopically-labeled compounds can generally be prepared by conventional techniques known to those skilled in the art or by processes analogous to those described herein, using an appropriate isotopically-labeled reagent in place of the non-labeled reagent otherwise employed.

“Treatment” as used herein, covers any administration or application of a therapeutic for disease in a human, and includes inhibiting disease progression of the disease or one or more disease symptoms, slowing the disease or its progression or one or more of its symptoms, arresting its development, partially or fully relieving the disease or one or more of its symptoms, or preventing a recurrence of one or more symptoms of the disease.

The terms “subject” and “patient” are used interchangeably herein to refer to a human unless specifically stated otherwise.

“Inhibitors of DGKα and/or DGKζ” refers to “inhibitors of DGKα and/or DGKζ enzyme activity,” both of which refer to inhibitors of human DGKα and/or human DGKζ, such as DGKα having the amino acid sequence shown in SEQ ID NO: 2, or the amino acid sequence shown in SEQ ID NO: 2 without the amino acids that are not naturally present in DGKα (e.g., the His tail or certain N-terminal amino acids) and DGKζ having the amino acid sequence shown in SEQ ID NO: 4, or the amino acid sequence shown in SEQ ID NO: 4 without the amino acids that are not naturally present in DGKζ (e.g., the His tail or certain N-terminal amino acids).

A target protein as used herein, e.g., DGK, PD-1, PD-L1 and CTLA4, refers to the human target protein, unless specifically indicated otherwise or the context clearly indicates otherwise. For example, “mouse DGK” refers to the mouse version of DGK, as it specifically indicates it.

“PD1” is used interchangeably with “PD-1.”

“CTLA4” is used interchangeably with “CTLA-4.” The term “effective amount” or “therapeutically effective amount” refers to an amount of a drug effective for treatment of a disease or disorder in a subject, such as to partially or fully relieve one or more symptoms. In some embodiments, an effective amount refers to an amount effective, at dosages and for periods of time necessary, to achieve the desired therapeutic or prophylactic result.

The term “cancer” is used herein to refer to a group of cells that exhibit abnormally high levels of proliferation and growth. A cancer may be benign (also referred to as a benign tumor), pre-malignant, or malignant. Cancer cells may be solid cancer cells or leukemic cancer cells. Examples of cancers applicable to methods of treatment herein include but are not limited to, carcinoma, lymphoma, blastoma, sarcoma, and leukemia. More particular nonlimiting examples of such cancers include squamous cell cancer, small-cell lung cancer, pituitary cancer, esophageal cancer, astrocytoma, soft tissue sarcoma, non-small cell lung cancer (including squamous cell non-small cell lung cancer), adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cell carcinoma, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, and various types of head and neck cancer (including squamous cell carcinoma of the head and neck).

The term “tumor growth” is used herein to refer to proliferation or growth by a cell or cells that comprise a cancer that leads to a corresponding increase in the size or extent of the cancer.

Administration “in combination with” one or more further therapeutic agents includes simultaneous (concurrent) and consecutive (sequential) administration in any order.

Methods of Treatment

Provided herein are methods of treating a proliferative disease, such as cancer, or a viral infection, or more generally, a disease, disorder or condition that benefits from the stimulation of the immune system, as well as any disease, disorder or condition that can be prevented, ameliorated, or cured by inhibiting DGKα and/or DGKζ enzyme activity, comprising administering to a subject in need thereof, a therapeutically effective amount of (i) an inhibitor of DGKα and/or DGKζ and (ii) an antagonist of the PD1/PD-L1 axis (e.g., an antagonist of human PD1 or human PD-L1) and/or an antagonist of human CTLA4. Provided herein are methods of treating a proliferative disease, such as cancer, or a viral infection, or more generally, a disease, disorder or condition that benefits from the stimulation of the immune system, as well as any disease, disorder or condition that can be prevented, ameliorated, or cured by inhibiting DGKα and/or DGKζ enzyme activity, comprising administering to a subject in need thereof, a therapeutically effective amount of (i) an inhibitor of DGKα and/or DGKζ and (ii) an antagonist of the PD1/PD-L1 axis (e.g., an antagonist of human PD1 or human PD-L1). Provided herein are methods of treating a proliferative disease, such as cancer, or a viral infection, or more generally, a disease, disorder or condition that benefits from the stimulation of the immune system, as well as any disease, disorder or condition that can be prevented, ameliorated, or cured by inhibiting DGKα and/or DGKζ enzyme activity, comprising administering to a subject in need thereof, a therapeutically effective amount of (i) an inhibitor of DGKα and/or DGKζ and an antagonist of human CTLA4. In certain embodiments, treating a proliferative disease, such as cancer, or a viral infection, or more generally, a disease, disorder or condition that benefits from the stimulation of the immune system, as well as any disease, disorder or condition that can be prevented, ameliorated, or cured by inhibiting DGKα and/or DGKζ enzyme activity, comprises administering to a subject in need thereof, a therapeutically effective amount of (i) an inhibitor of DGKα and/or DGKζ and (ii) an antagonist of the PD1/PD-L1 axis (e.g., an antagonist of human PD1 or human PD-L1) and an antagonist of human CTLA4.

Administration of (i) an inhibitor of DGKα and/or DGKζ and (ii) an antagonist of the PD1/PD-L1 axis (e.g., an antagonist of human PD1 or human PD-L1) and/or an antagonist of human CTLA4 can be simultaneous of sequential. For example, in certain embodiments, a method of treating cancer or a disease that can be treated by increasing an immune response, comprises administering to a subject in need thereof first an inhibitor of DGKα and/or DGKζ and then, later (e.g., 6 hours, 12 hours, 24 hours, 2 days, 3 days or more later), administering an antagonist of the PD1/PD-L1 axis (e.g., an antagonist of human PD1 or human PD-L1) and/or an antagonist of human CTLA4. A method, e.g., a method of treating cancer, may comprise treating cancer or a disease that can be treated by increasing an immune response, comprises administering to a subject in need thereof first an antagonist of the PD1/PD-L1 axis (e.g., an antagonist of human PD1 or human PD-L1) and/or an antagonist of human CTLA4, and then, later (e.g., 6 hours, 12 hours, 24 hours, 2 days, 3 days or more later), administering an inhibitor of DGKα and/or DGKζ.

A method, e.g., a method of treating cancer, may comprise administering first an inhibitor of DGKα and/or DGKζ, and then later (e.g., 6 hours, 12 hours, 24 hours, 2 days, 3 days or more later), administering an antagonist of the PD1/PD-L1 axis (e.g., an antagonist of human PD1 or human PD-L1) and at the same time, administering an antagonist of human CTLA4. A method, e.g., a method of treating cancer, may comprise administering first an inhibitor of DGKα and/or DGKζ, and then later (e.g., 6 hours, 12 hours, 24 hours, 2 days, 3 days or more later), administering an antagonist of the PD1/PD-L1 axis (e.g., an antagonist of human PD1 or human PD-L1) and at the same time, administering an antagonist of human CTLA4. A method, e.g., a method of treating cancer, may comprise administering first an antagonist of the PD1/PD-L1 axis (e.g., an antagonist of human PD1 or human PD-L1) and at the same time, administering an antagonist of human CTLA4, and then later (e.g., 6 hours, 12 hours, 24 hours, 2 days, 3 days or more later), administering an inhibitor of DGKα and/or DGKζ.

The methods described herein may be used for treating a cancer, such as an advanced cancer, metastatic cancer, solid tumors, advanced solid tumors, hematological tumors, cancers that are refractory to checkpoint inhibitors (or checkpoint antagonists), or those that have progressed after treatment with a checkpoint inhibitor.

Non-limiting examples of cancers for treatment include squamous cell carcinoma, small-cell lung cancer, non-small cell lung cancer, squamous non-small cell lung cancer (NSCLC), nonsquamous NSCLC, glioma, gastrointestinal cancer, renal cancer (e.g., clear cell carcinoma), ovarian cancer, liver cancer, colorectal cancer, endometrial cancer, kidney cancer (e.g., renal cell carcinoma (RCC)), prostate cancer (e.g., hormone refractory prostate adenocarcinoma), thyroid cancer, neuroblastoma, pancreatic cancer, glioblastoma (glioblastoma multiforme), cervical cancer, stomach cancer, bladder cancer, hepatoma, breast cancer, colon carcinoma, and head and neck cancer (or carcinoma), gastric cancer, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, T-cell lymphoma, environmentally-induced cancers including those induced by asbestos, virus-related cancers or cancers of viral origin (e.g., human papilloma virus (HPV-related or -originating tumors)), and hematologic malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CML), undifferentiated AML (MO), myeloblastic leukemia (M1), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NHL), B cell hematologic malignancy, e.g., B-cell lymphomas, T-cell lymphomas, lymphoplasmacytoid lymphoma, monocytoid B-cell lymphoma, mucosa-associated lymphoid tissue (MALT) lymphoma, anaplastic (e.g., Ki 1+) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukaemia (T-Lbly/T-ALL), peripheral T-cell lymphoma, lymphoblastic lymphoma, post-transplantation lymphoproliferative disorder, true histiocytic lymphoma, primary central nervous system lymphoma, primary effusion lymphoma, B cell lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of the central and peripheral nervous, including astrocytoma, schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) of the T-cell type; a/d T-NHL hepatosplenic lymphoma; peripheral/post-thymic T cell lymphoma (pleomorphic and immunoblastic subtypes); angiocentric (nasal) T-cell lymphoma; cancer of the head or neck, renal cancer, rectal cancer, cancer of the thyroid gland; acute myeloid lymphoma, as well as any combinations of said cancers. The methods described herein can also be used for treatment of metastatic cancers, unresectable, refractory cancers (e.g., cancers refractory to previous immunotherapy, e.g., with a blocking CTLA-4 or PD-1 antibody), and/or recurrent cancers.

In certain embodiments, a combination treatment described herein is administered to patients having a cancer that has exhibited an inadequate response to, or progressed on, a prior treatment, e.g., a prior treatment with an immuno-oncology or immunotherapy drug. In some embodiments, the cancer is refractory or resistant to a prior treatment, either intrinsically refractory or resistant (e.g., refractory to a PD-1 pathway antagonist), or a resistance or refractory state is acquired. For example, a combination treatment described herein may be administered to subjects who are not responsive or not sufficiently responsive to a first therapy or who have disease progression following treatment, e.g., anti-PD-1 pathway antagonist treatment, either alone or in combination with another therapy (e.g., with an anti-PD-1 pathway antagonist therapy). In other embodiments, a combination treatment described herein is administered to patients who have not previously received (i.e., been treated with) an immuno-oncology agent, e.g., a PD-1 pathway antagonist.

The combination treatments may further comprise one or more additional treatments, such as radiation, surgery or chemotherapy.

Methods described herein can also be used to treat patients that have been exposed to particular toxins or pathogens, such as those having an infectious disease. Accordingly, this disclosure also contemplates methods of treating an infectious disease in a subject comprising administering to the subject a combination treatment as described herein, such that the subject is treated for the infectious disease. Similar to its application to tumors as discussed above, the combination treatment can be used alone, or as an adjuvant, in combination with vaccines, to stimulate the immune response to pathogens, toxins, and self-antigens. Examples of pathogens for which this therapeutic approach might be particularly useful, include pathogens for which there is currently no effective vaccine, or pathogens for which conventional vaccines are less than completely effective. These include, but are not limited to HIV, Hepatitis (A, B, & C), Influenza, Herpes, Giardia, Malaria, Leishmania, Staphylococcus aureus, Pseudomonas aeruginosa. Combination treatment can be useful against established infections by agents such as HIV that present altered antigens over the course of the infections.

Some examples of pathogenic viruses causing infections that may be treatable by methods described herein include HIV, hepatitis (A, B, or C), herpes virus (e.g., VZV, HSV-1, HAV-6, HSV-II, and CMV, Epstein Barr virus), adenovirus, influenza virus, flaviviruses, echovirus, rhinovirus, coxsackie virus, coronavirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Some examples of pathogenic bacteria causing infections that may be treatable by methods described herein include chlamydia, rickettsial bacteria, mycobacteria, staphylococci, streptococci, pneumonococci, meningococci and gonococci, klebsiella, proteus, serratia, pseudomonas, legionella, diphtheria, salmonella, bacilli, cholera, tetanus, botulism, anthrax, plague, leptospirosis, and Lymes disease bacteria.

Some examples of pathogenic fungi causing infections that may be treatable by methods described herein include Candida (albicans, krusei, glabrata, tropicalis, etc.), Cryptococcus neoformans, Aspergillus (fumigatus, niger, etc.), Genus Mucorales (mucor, absidia, rhizopus), Sporothrix schenkii, Blastomyces dermatitidis, Paracoccidioides brasiliensis, Coccidioides immitis and Histoplasma capsulatum.

Some examples of pathogenic parasites causing infections that may be treatable by methods described herein include Entamoeba histolytica, Balantidium coli, Naegleriafowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondii, and Nippostrongylus brasiliensis.

In all of the above methods, the combination treatment can be combined with other forms of immunotherapy, e.g., those described herein, such as cytokine treatment (e.g., interferons, GM-CSF, G-CSF, IL-2), or bispecific antibody therapy, which may provide for enhanced presentation of tumor antigens (see, e.g., Holliger (1993) Proc. Natl. Acad. Sci. USA 90:6444-6448; Poljak (1994) Structure 2: 1121-1123).

In certain embodiments, a method comprises administering to a subject in need thereof, e.g., a subject having cancer, an inhibitor of DGKα and/or DGKζ in combination with an antagonist of the PD1/PD-L1 axis and/or an antagonist of CTLA4 and also an agent that inhibits CD4+ T cells and/or an agent that boosts CD8+ T cells. In certain embodiments, agents that inhibit CD4+ T cells and agents that boost CD8+ T cells may be agents that act locally in the tumor environment.

Exemplary Inhibitors of DGKα and/or DGKζ Enzyme Activity

In certain embodiments, an inhibitor of DGKα and/or DGKζ is an inhibitor of DGKα. In certain embodiments, an inhibitor of DGKα and/or DGKζ is an inhibitor of DGKζ. In certain embodiments, an inhibitor of DGKα and/or DGKζ inhibits both enzymes. The level of enzyme inhibition may be measured as further described herein. In certain embodiments, an inhibitor of DGKα and/or DGKζ is not a significant inhibitor of other DGK enzymes.

In certain embodiments, an inhibitor of DGKα and/or DGKζ increases an immune response, such as by increasing T cell activity. For example, an inhibitor of DGKα and/or DGKζ may increase primary T cell signaling, as evidenced, e.g., by an increase in pERK/pPKC signaling, which may be measured as further described herein. In certain embodiments, an inhibitor of DGKα and/or DGKζ has one or more of the following properties: (i) it lowers the threshold for antigen stimulation; (ii) increases CTL effector function; and (iii) enhances tumor cell killing. When an inhibitor of DGKα and/or DGKζ enhances tumor cell killing, this activity may be dependent on CD8+ T cells, as shown, e.g., in the CT26 animal model. When an inhibitor of DGKα and/or DGKζ enhances tumor cell killing, this activity may be dependent on NK cells, as shown, e.g., in the CT26 animal model. When an inhibitor of DGKα and/or DGKζ enhances tumor cell killing, this activity may be dependent on CD8+ T cells and NK cells, as shown, e.g., in the CT26 animal model. When an inhibitor of DGKα and/or DGKζ enhances tumor cell killing, this activity may be enhanced by CD4 cell depletion, e.g., in the CT-26 animal model. In certain embodiments, an inhibitor of DGKα and/or DGKζ enhances AH1+ Tetramer antigen presentation in the CT-26 animal model. An inhibitor of DGKα and/or DGKζ preferably includes one or more of the above cited properties, and may include all of them. These presence of these properties can be determined by conducting assays described herein, such as in the section entitled “Biological assays” in the Examples.

A method of treating a disease, such as cancer, may comprise administering to a subject in need thereof an antagonist of the PD1/PD-L1 axis and/or an antagonist of CTLA4 and an inhibitor of DGKα and/or DGKζ that is a compound of Formula (I):

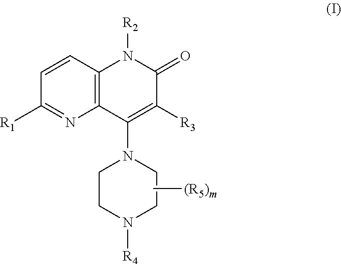

or a pharmaceutically acceptable salt thereof, wherein:
R₁ is H, F, Cl, Br, —CN, C₁₋₃ alkyl substituted with zero to 4 R₁ₐ, C₃₋₄ cycloalkyl substituted with zero to 4 R₁ₐ, C₁₋₃ alkoxy substituted with zero to 4 R₁ₐ, —NRₐRₐ, —S(O)ₙRₑ, or —P(O)RₑRₑ;
each R₁ₐ is independently F, Cl, —CN, —OH, —OCH₃, or —NRₐRₐ;
each Rₐ is independently H or C₁₋₃ alkyl;
each Rₑ is independently C₃₋₄ cycloalkyl or C₁₋₃ alkyl substituted with zero to 4 Ria;
R₂ is H, C₁₋₃ alkyl substituted with zero to 4 R₂ₐ, or C₃₋₄ cycloalkyl substituted with zero to 4 R₂ₐ;
each R₂ₐ is independently F, Cl, —CN, —OH, —O(C₁₋₂ alkyl), C₃₋₄ cycloalkyl, C₃₋₄ alkenyl, or C₃₋₄ alkynyl;
R₃ is H, F, Cl, Br, —CN, C₁₋₃ alkyl, C₁₋₂ fluoroalkyl, C₃₋₄ cycloalkyl, C₃₋₄ fluorocycloalkyl, or —NO₂;
R₄ is —CH₂R₄ₐ, —CH₂CH₂R₄ₐ, —CH₂CHR₄ₐR₄ₐ, —CHR₄ₐR_{4b}, or —CR₄ₐR_{4b}R_{4c}; R₄ₐ and R_{4b} are independently:
   (i) C₁₋₆ alkyl substituted with zero to 4 substituents independently selected from F, Cl, —CN, —OH, —OCH₃, —SCH₃, C₁₋₃ fluoroalkoxy, —NRₐRₐ, —S(O)₂Rₑ, or —NRₐS(O)₂Rₑ;
   (ii) C₃₋₆ cycloalkyl, heterocyclyl, phenyl, or heteroaryl, each substituted with zero to 4 substituents independently selected from F, Cl, Br, —CN, —OH, C₁₋₆ alkyl, C₁₋₃ fluoroalkyl, C₁₋₄ hydroxyalkyl, —(CH₂)₁₋₂O(C₁₋₃ alkyl), C₁₋₄ alkoxy, —O(C₁₋₄ hydroxyalkyl), —O(CH)₁₋₃O(C₁₋₃ alkyl), C₁₋₃ fluoroalkoxy, —O(CH)₁₋₃NR_{c}R_{c}, —OCH₂CH═CH₂, —OCH₂C═CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₃ alkyl)₂, —S(O)₂(C₁₋₃ alkyl), —O(CH₂)₁₋₂(C₃₋₆ cycloalkyl), —O(CH₂)₁₋₂(morpholinyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, and R_{d}; or
   (iii) C₁₋₄ alkyl substituted with one cyclic group selected from C₃₋₆ cycloalkyl, heterocyclyl, aryl, and heteroaryl, said cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C═CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₆ cycloalkyl;
or R₄ₐ and R_{4b} together with the carbon atom to which they are attached form a C₃₋₆ cycloalkyl or a 3- to 6-membered heterocyclyl, each substituted with zero to 3 R_{f};
each R_{f} is independently F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C═CH, —NR_{c}R_{c}, or a cyclic group selected from C₃₋₆ cycloalkyl, 3- to 6-membered heterocyclyl, phenyl, monocyclic heteroaryl, and bicyclic heteroaryl, each cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, and —NR_{c}R_{c};
R_{4c} is C₁₋₆ alkyl or C₃₋₆ cycloalkyl, each substituted with zero to 4 substituents independently selected from F, Cl, —OH, C₁₋₂ alkoxy, C₁₋₂ fluoroalkoxy, and —CN;
R_{4d} is —OCH₃;
each R_{c} is independently H or C₁₋₂ alkyl;
R_{d} is phenyl substituted with zero to 1 substituent selected from F, Cl, —CN, —CH₃, and —OCH₃;
each R₅ is independently —CN, C₁₋₆ alkyl substituted with zero to 4 R_{g}, C₂₋₄ alkenyl substituted with zero to 4 R_{g}, C₂₋₄ alkynyl substituted with zero to 4 R_{g}, C₃₋₄ cycloalkyl substituted with zero to 4 R_{g}, phenyl substituted with zero to 4 R_{g}, oxadiazolyl substituted with zero to 3 R_{g}, pyridinyl substituted with zero to 4 R_{g}, —(CH₂)₁₋₂(heterocyclyl substituted with zero to 4 R_{g}), —(CH₂)₁₋₂NR_{c}C(O)(C₁₋₄ alkyl), —(CH₂)₁₋₂NR_{c}C(O)O(C₁₋₄ alkyl), —(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl);
each R_{g} is independently F, Cl, —CN, —OH, C₁₋₃ alkoxy, C₁₋₃ fluoroalkoxy, —O(CH₂)₁₋₂O(C₁₋₂ alkyl), or —NR_{c}R_{c};
m is zero, 1, 2, or 3; and
n is zero, 1, or 2.

A method of treating a disease, such as cancer, may comprise administering to a subject in need thereof an antagonist of the PD1/PD-L1 axis and/or an antagonist of CTLA4 and an inhibitor of DGKα and/or DGKζ that is a compound of Formula (I) or a pharmaceutically acceptable salt thereof, wherein:
R₁ is H, F, Cl, Br, —CN, C₁₋₃ alkyl substituted with zero to 4 R₁ₐ, cyclopropyl substituted with zero to 3 R₁ₐ, C₁₋₃ alkoxy substituted with zero to 3 R₁ₐ, —NRₐRₐ, —S(O)ₙCH₃, or —P(O)(CH₃)₂;
each R₁ₐ is independently F, Cl, or —CN;
each Rₐ is independently H or C₁₋₃ alkyl;
R₂ is H or C₁₋₂ alkyl substituted with zero to 2 R₂ₐ;
each R₂ₐ is independently F, Cl, —CN, —OH, —O(C₁₋₂ alkyl), cyclopropyl, C₃₋₄ alkenyl, or C₃₋₄ alkynyl;
R₃ is H, F, Cl, Br, —CN, C₁₋₂ alkyl, —CF₃, cyclopropyl, or —NO₂;
R₄ₐ and R_{4b} are independently:
   (i) C₁₋₄ alkyl substituted with zero to 4 substituents independently selected from F, Cl, —CN, —OH, —OCH₃, —SCH₃, C₁₋₃ fluoroalkoxy, and —NRₐRₐ;
   (ii) C₃₋₆ cycloalkyl, heterocyclyl, phenyl, or heteroaryl, each substituted with zero to 4 substituents independently selected from F, Cl, Br, —CN, —OH, C₁₋₆ alkyl, C₁₋₃ fluoroalkyl, —CH₂OH, —(CH₂)₁₋₂O(C₁₋₂ alkyl), C₁₋₄ alkoxy, —O(C₁₋₄ hydroxyalkyl), —O(CH)₁₋₂O(C₁₋₂ alkyl), C₁₋₃ fluoroalkoxy, —O(CH)₁₋₂NR_{c}R_{c}, —OCH₂CH═CH₂, —OCH₂C═CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₂ alkyl)₂, —S(O)₂(C₁₋₃ alkyl), —O(CH₂)₁₋₂(C₃₋₄ cycloalkyl), —O(CH₂)₁₋₂(morpholinyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, and R_{d}; or
   (iii) C₁₋₃ alkyl substituted with one cyclic group selected from C₃₋₆ cycloalkyl, heterocyclyl, phenyl, and heteroaryl, said cyclic group substituted with zero to 3 substituents independently selected from F, Cl, Br, —OH, —CN, C₁₋₃ alkyl, C₁₋₂ fluoroalkyl, C₁₋₃ alkoxy, C₁₋₂ fluoroalkoxy, —OCH₂CH═CH₂, —OCH₂C═CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₄ cycloalkyl;
or R₄ₐ and R_{4b} together with the carbon atom to which they are attached, form a C₃₋₆ cycloalkyl or a 3- to 6-membered heterocyclyl, each substituted with zero to 3 R_{f};
each R_{f} is independently F, Cl, Br, —OH, —CN, C₁₋₄ alkyl, C₁₋₂ fluoroalkyl, C₁₋₃ alkoxy, C₁₋₂ fluoroalkoxy, —OCH₂CH═CH₂, —OCH₂C═CH, —NR_{c}R_{c}, or a cyclic group selected from C₃₋₆ cycloalkyl, 3- to 6-membered heterocyclyl, phenyl, monocyclic heteroaryl, and bicyclic heteroaryl, each cyclic group substituted with zero to 3 substituents independently selected from F, Cl, Br, —OH, —CN, C₁₋₄ alkyl, C₁₋₂ fluoroalkyl, C₁₋₃ alkoxy, C₁₋₂ fluoroalkoxy, and —NR_{c}R_{c};
R_{4c} is C₁₋₄ alkyl or C₃₋₆ cycloalkyl, each substituted with zero to 4 substituents independently selected from F, Cl, —OH, C₁₋₂ alkoxy, C₁₋₂ fluoroalkoxy, and —CN;
and each R₅ is independently —CN, C₁₋₅ alkyl substituted with zero to 4 R_{g}, C₂₋₃ alkenyl substituted with zero to 4 R_{g}, C₂₋₃ alkynyl substituted with zero to 4 R_{g}, C₃₋₄ cycloalkyl substituted with zero to 4 R_{g}, phenyl substituted with zero to 3 R_{g}, oxadiazolyl substituted with zero to 3 R_{g}, pyridinyl substituted with zero to 3 R_{g}, —(CH₂)₁₋₂(heterocyclyl substituted with zero to 4 R_{g}), —(CH₂)₁₋₂NR_{c}C(O)(C₁₋₄ alkyl), —(CH₂)₁₋₂NR_{c}C(O)O(C₁₋₄ alkyl), —(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl).

A method of treating a disease, such as cancer, may comprise administering to a subject in need thereof an antagonist of the PD1/PD-L1 axis and/or an antagonist of CTLA4 and an inhibitor of DGKα and/or DGKζ that is a compound of Formula (I) or a pharmaceutically acceptable salt thereof having the structure:

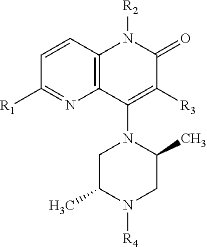

wherein:

### R₁ is —CN;

R₂ is —CH₃;

### R₃ is H, F, or —CN;

### R₄ is:

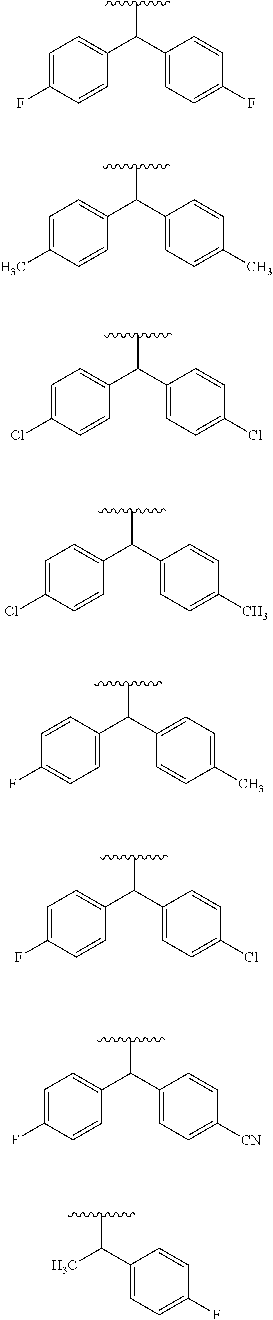

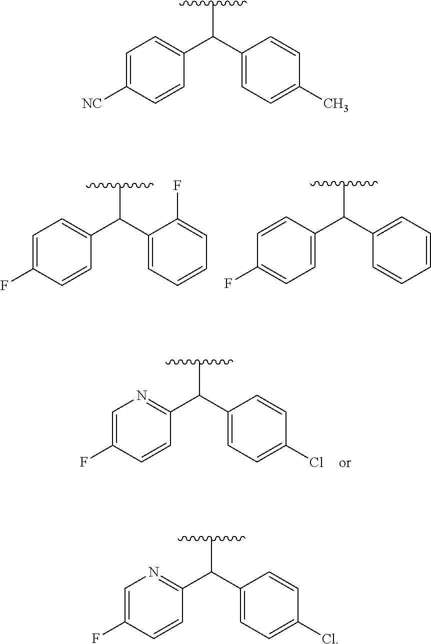

A method of treating a disease, such as cancer, may comprise administering to a subject in need thereof an antagonist of the PD1/PD-L1 axis and/or an antagonist of CTLA4 and an inhibitor of DGKα and/or DGKζ that is a compound of Formula (I) or a pharmaceutically acceptable salt thereof having one the following structure or formula (or an isomer thereof):
Methyl 1-(bis(4-fluorophenyl)methyl)-4-(6-cyano-1-methyl-2-oxo-1,2-dihydro-1,5-naphthyridin-4-yl)piperazine-2-carboxylate

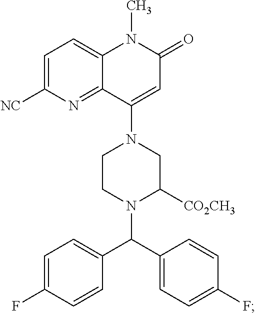 [SMILES: COC(=O)C1CN(c2cc(=O)n(C)c3ccc(C#N)nc23)CCN1C(c1ccc(F)cc1)c1ccc(F)cc1]

4-((2R,5S)-4-(bis(4-fluorophenyl)methyl)-2,5-dimethylpiperazin-1-yl)-6-bromo-1-methyl-2-oxo-1,2-dihydro-1,5-naphthyridine-3-carbonitrile

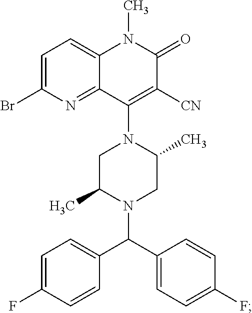 [SMILES: C[C@@H]1CN(C(c2ccc(F)cc2)c2ccc(F)cc2)[C@@H](C)CN1c1c(C#N)c(=O)n(C)c2ccc(Br)nc12]

(R)-8-(4-(bis(4-fluorophenyl)methyl)-3-methylpiperazin-1-yl)-5-methyl-6-oxo-5,6-dihydro-1,5-naphthyridine-2,7-dicarbonitrile

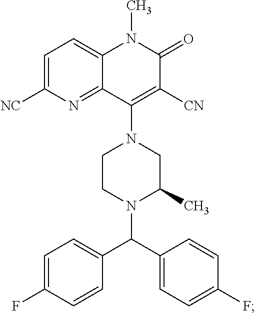 [SMILES: C[C@@H]1CN(c2c(C#N)c(=O)n(C)c3ccc(C#N)nc23)CCN1C(c1ccc(F)cc1)c1ccc(F)cc1]

8-[(2S,5R)-4-[(4-chlorophenyl)(5-methylpyridin-2-yl)methyl]-2,5-dimethylpiperazin-1-yl]-5-methyl-6-oxo-5,6-dihydro-1,5-naphthyridine-2-carbonitrile

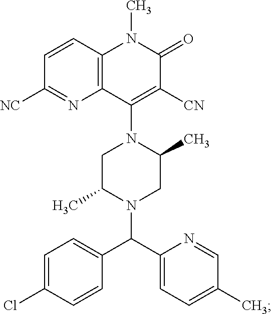 [SMILES: Cc1ccc(C(c2ccc(Cl)cc2)N2C[C@H](C)N(c3c(C#N)c(=O)n(C)c4ccc(C#N)nc34)C[C@H]2C)nc1]

4-[(2S,5R)-4-[(4-chlorophenyl)(4-fluorophenyl)methyl]-2,5-dimethylpiperazin-1-yl]-6-methoxy-1-methyl-1,2-dihydro-1,5-naphthyridin-2-one

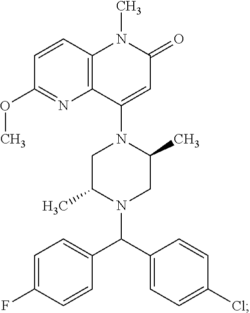 [SMILES: COc1ccc2c(n1)c(N1C[C@@H](C)N(C(c3ccc(F)cc3)c3ccc(Cl)cc3)C[C@@H]1C)cc(=O)n2C]

8-[(2S,5R)-4-{[2-(difluoromethyl)-4-fluorophenyl]methyl}-2,5-dimethylpiperazin-1-yl]-5-methyl-6-oxo-5,6-dihydro-1,5-naphthyridine-2-carbonitrile

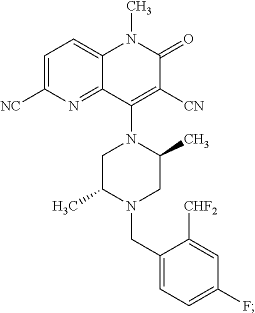 [SMILES: C[C@@H]1CN(c2c(C#N)c(=O)n(C)c3ccc(C#N)nc23)[C@@H](C)CN1Cc1ccc(F)cc1C(F)F]

8-[(2S,5R)-4-[(4-fluorophenyl)(4-methylphenyl)methyl]-2,5-dimethylpiperazin-1-yl]-5-methyl-6-oxo-5,6-dihydro-1,5-naphthyridine-2-carbonitrile

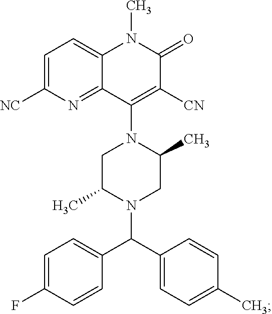 [SMILES: Cc1ccc(C(c2ccc(F)cc2)N2C[C@H](C)N(c3c(C#N)c(=O)n(C)c4ccc(C#N)nc34)C[C@H]2C)cc1]

8-[(2S,5R)-4-[1-(2,6-difluorophenyl)ethyl]-2,5-dimethylpiperazin-1-yl]-5-methyl-6-oxo-5,6-dihydro-1,5-naphthyridine-2-carbonitrile

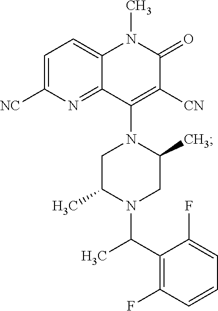 [SMILES: CC(c1c(F)cccc1F)N1C[C@H](C)N(c2c(C#N)c(=O)n(C)c3ccc(C#N)nc23)C[C@H]1C]

8-((3R)-4-((4-chlorophenyl)(5-fluoropyridin-2-yl)methyl)-3-methylpiperazin-1-yl)-5-methyl-6-oxo-5,6-dihydro-1,5-naphthyridine-2,7-dicarbonitrile

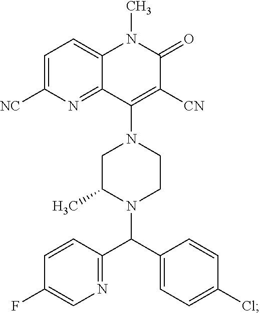 [SMILES: C[C@@H]1CN(c2c(C#N)c(=O)n(C)c3ccc(C#N)nc23)CCN1C(c1ccc(Cl)cc1)c1ccc(F)cn1]

8-(4-(bis(4-fluorophenyl)methyl)piperazin-1-yl)-5-methyl-7-nitro-6-oxo-5,6-dihydro-1,5-naphthyridine-2-carbonitrile

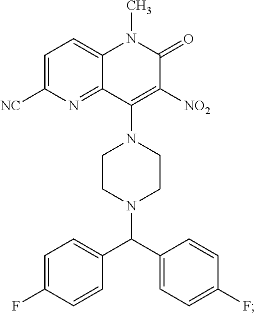 [SMILES: Cn1c(=O)c([N+](=O)[O-])c(N2CCN(C(c3ccc(F)cc3)c3ccc(F)cc3)CC2)c2nc(C#N)ccc21]

and
8-[(2S,5R)-4-[bis(4-methylphenyl)methyl]-2,5-dimethylpiperazin-1-yl]-5-methyl-6-oxo-5,6-dihydro-1,5-naphthyridine-2-carbonitrile

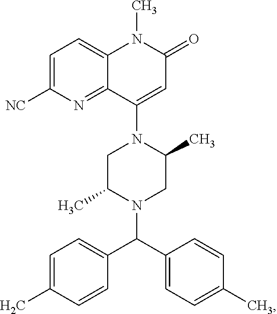 [SMILES: Cc1ccc(C(c2ccc(C)cc2)N2C[C@H](C)N(c3cc(=O)n(C)c4ccc(C#N)nc34)C[C@H]2C)cc1]

A method of treating a disease, such as cancer, may comprise administering to a subject in need thereof an antagonist of the PD1/PD-L1 axis and/or an antagonist of CTLA4 and an inhibitor of DGKα and/or DGKζ that is a compound of Formula (II):

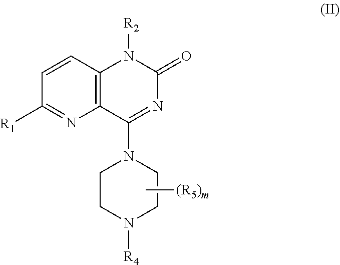

or a salt thereof, wherein:
R₁ is H, F, Cl, Br, —CN, —OH, C₁₋₃ alkyl substituted with zero to 4 R₁ₐ, C₃₋₄ cycloalkyl substituted with zero to 4 R₁ₐ, C₁₋₃ alkoxy substituted with zero to 4 R₁ₐ, —NRₐRₐ, —S(O)ₙRₑ, or —P(O)RₑRₑ;
each R₁ₐ is independently F, Cl, —CN, —OH, —OCH₃, or —NRₐRₐ;
each Rₐ is independently H or C₁₋₃ alkyl;
each Rₑ is independently C₃₋₄ cycloalkyl or C₁₋₃ alkyl substituted with zero to 4 R₁ₐ;
R₂ is H, C₁₋₃ alkyl substituted with zero to 4 R₂ₐ, or C₃₋₄ cycloalkyl substituted with zero to 4 R₂ₐ;
each R₂ₐ is independently F, Cl, —CN, —OH, —O(C₁₋₂ alkyl), C₃₋₄ cycloalkyl, C₃₋₄ alkenyl, or C₃₋₄ alkynyl;
R₄ is —CH₂R₄ₐ, —CH₂CH₂R₄ₐ, —CH₂CHR₄ₐR_{4d}, —CHR₄ₐR_{4b}, or —CR₄ₐR_{4b}R_{4c}; R₄ₐ and R_{4b} are independently:
   (i) —CN or C₁₋₆ alkyl substituted with zero to 4 substituents independently selected from F, Cl, —CN, —OH, —OCH₃, —SCH₃, C₁₋₃ fluoroalkoxy, —NRₐRₐ, —S(O)₂Rₑ, or —NRₐS(O)₂Rₑ;
   (ii) C₃₋₆ cycloalkyl, 4- to 10-membered heterocyclyl, phenyl, or 5- to 10-membered heteroaryl, each substituted with zero to 4 substituents independently selected from F, Cl, Br, —CN, —OH, C₁₋₆ alkyl, C₁₋₃ fluoroalkyl, C₁-2 bromoalkyl, C₁₋₂ cyanoalkyl, C₁₋₄ hydroxyalkyl, —(CH₂)₁₋₂O(C₁₋₃ alkyl), C₁₋₄ alkoxy, C₁₋₃ fluoroalkoxy, C₁₋₃ cyanoalkoxy, —O(C₁₋₄ hydroxyalkyl), —O(CRₓRₓ)₁₋₃O(C₁₋₃ alkyl), C₁₋₃ fluoroalkoxy, —O(CH₂)₁₋₃NR_{c}R_{c}, —OCH₂CH═CH₂, —OCH₂C═CH, —C(O)(C₁₋₄ alkyl), —C(O)OH, —C(O)O(C₁₋₄ alkyl), —NR_{c}R_{c}, —CH₂NRₐRₐ, —NRₐS(O)₂(C₁₋₃ alkyl), —NRₐC(O)(C₁₋₃ alkyl), —(CRₓRₓ)₀₋₂NRₐC(O)O(C₁₋₄ alkyl), —P(O)(C₁₋₃ alkyl)₂, —S(O)₂(C₁₋₃ alkyl), —(CRₓRₓ)₁₋₂(C₃₋₄ cycloalkyl), —(CRₓRₓ)₁₋₂(morpholinyl), —(CRₓRₓ)₁₋₂(difluoromorpholinyl), —(CRₓRₓ)₁₋₂(dimethylmorpholinyl), —(CRₓRₓ)₁₋₂(oxaazabicyclo[2.2.1]heptanyl), (CRₓRₓ)₁₋₂(oxaazaspiro[3.3]heptanyl), —(CRₓRₓ)₁₋₂(methylpiperazinonyl), —(CRₓRₓ)₁₋₂(acetylpiperazinyl), —(CRₓRₓ)₁₋₂(piperidinyl), —(CRₓRₓ)₁₋₂(difluoropiperidinyl), —(CRₓRₓ)₁₋₂(methoxypiperidinyl), —(CRₓRₓ)₁₋₂(hydroxypiperidinyl), —O(CRₓRₓ)₀₋₂(C₃₋₆ cycloalkyl), —O(CRₓRₓ)₀₋₂(methylcyclopropyl), —O(CRₓRₓ)₀₋₂((ethoxycarbonyl)cyclopropyl), —O(CRₓRₓ)₀₋₂(oxetanyl), —O(CRₓRₓ)₀₋₂(methylazetidinyl), —O(CRₓRₓ)₀₋₂(tetrahydropyranyl), —O(CRₓRₓ)₁₋₂(morpholinyl), —O(CRₓRₓ)₀₋₂(thiazolyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, dioxolanyl, pyrrolidinonyl, and R_{d}; or
   (iii) C₁₋₄ alkyl substituted with one cyclic group selected from C₃₋₆ cycloalkyl, 4- to 10-membered heterocyclyl, mono- or bicyclic aryl, or 5- to 10-membered heteroaryl, said cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C═CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₆ cycloalkyl;
or R₄ₐ and R_{4b} together with the carbon atom to which they are attached form a C₃₋₆ cycloalkyl or a 3- to 6-membered heterocyclyl, each substituted with zero to 3 R_{f};
each R_{f} is independently F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C—CH, —NR_{c}R_{c}, or a cyclic group selected from C₃₋₆ cycloalkyl, 3- to 6-membered heterocyclyl, phenyl, monocyclic heteroaryl, and bicyclic heteroaryl, each cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, and —NR_{c}R_{c};
R_{4c} is C₁₋₆ alkyl or C₃₋₆ cycloalkyl, each substituted with zero to 4 substituents independently selected from F, Cl, —OH, C₁₋₂ alkoxy, C₁₋₂ fluoroalkoxy, and —CN;
R_{4d} is —OCH₃;
each R_{c} is independently H or C₁₋₂ alkyl;
R_{d} is phenyl substituted with zero to 1 substituent selected from F, Cl, —CN, —CH₃, and —OCH₃;
each R₅ is independently —CN, C₁₋₆ alkyl substituted with zero to 4 R_{g}, C₂₋₄ alkenyl substituted with zero to 4 R_{g}, C₂₋₄ alkynyl substituted with zero to 4 R_{g}, C₃₋₄ cycloalkyl substituted with zero to 4 R_{g}, phenyl substituted with zero to 4 R_{g}, oxadiazolyl substituted with zero to 3 R_{g}, pyridinyl substituted with zero to 4 R_{g}, —(CH₂)₁₋₂ (4- to 10-membered heterocyclyl substituted with zero to 4 R_{g}), —(CH₂)₁₋₂NR_{c}C(O)(C₁₋₄ alkyl), —(CH₂)₁₋₂NR_{c}C(O)O(C₁₋₄ alkyl), —(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl);
each R_{g} is independently F, Cl, —CN, —OH, C₁₋₃ alkoxy, C₁₋₃ fluoroalkoxy, —O(CH₂)₁₋₂O(C₁₋₂ alkyl), or —NR_{c}R_{c};
m is zero, 1, 2, or 3; and
n is zero, 1, or 2.

A method of treating a disease, such as cancer, may comprise administering to a subject in need thereof an antagonist of the PD1/PD-L1 axis and/or an antagonist of CTLA4 and an inhibitor of DGKα and/or DGKζ that is a compound of Formula (II) or a pharmaceutically acceptable salt thereof, wherein:
R₁ is H, F, Cl, Br, —CN, —OH, C₁₋₃ alkyl substituted with zero to 4 R₁ₐ, cyclopropyl substituted with zero to 3 R₁ₐ, C₁₋₃ alkoxy substituted with zero to 3 R₁ₐ, —NRₐRₐ, —S(O)ₙCH₃, or —P(O)(CH₃)₂;
R₂ is H or C₁₋₂ alkyl substituted with zero to 2 R₂ₐ;
each R₂ₐ is independently F, Cl, —CN, —OH, —O(C₁₋₂ alkyl), cyclopropyl, C₃₋₄ alkenyl, or C₃₋₄ alkynyl;
R₄ₐ and R_{4b} are independently:
   (i) —CN or C₁₋₄ alkyl substituted with zero to 4 substituents independently selected from F, Cl, —CN, —OH, —OCH₃, —SCH₃, C₁₋₃ fluoroalkoxy, and —NRₐRₐ;
   (ii) C₃₋₆ cycloalkyl, 4- to 10-membered heterocyclyl, phenyl, or 5- to 10-membered heteroaryl, each substituted with zero to 4 substituents independently selected from F, Cl, Br, —CN, —OH, C₁₋₆ alkyl, C₁₋₃ fluoroalkyl, C₁₋₂ bromoalkyl, C₁₋₂ cyanoalkyl, C₁₋₂ hydroxyalkyl, —CH₂NRₐRₐ, —(CH₂)₁₋₂O(C₁₋₂ alkyl), —(CH₂)₁₋₂NRₓC(O)O(C₁₋₂ alkyl), C₁₋₄ alkoxy, —O(C₁₋₄ hydroxyalkyl), —O(CRₓRₓ)₁₋₂O(C₁₋₂ alkyl), C₁₋₃ fluoroalkoxy, C₁₋₃ cyanoalkoxy, —O(CH₂)₁₋₂NR_{c}R_{c}, —OCH₂CH═CH₂, —OCH₂C—CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₂ alkyl)₂, —S(O)₂(C₁₋₃ alkyl), —(CH₂)₁₋₂(C₃₋₄ cycloalkyl), —CRₓRₓ(morpholinyl), —CRₓRₓ(difluoromorpholinyl), —CRₓRₓ(dimethylmorpholinyl), —CRₓRₓ(oxaazabicyclo[2.2.1]heptanyl), —CRₓRₓ(oxaazaspiro[3.3]heptanyl), —CRₓRₓ(methylpiperazinonyl), —CRₓRₓ(acetylpiperazinyl), —CRₓRₓ(piperidinyl), —CRₓRₓ(difluoropiperidinyl), —CRₓRₓ(methoxypiperidinyl), —CRₓRₓ(hydroxypiperidinyl), —O(CH₂)₀₋₂(C₃₋₄ cycloalkyl), —O(CH₂)₀₋₂(methylcyclopropyl), —O(CH₂)₀₋₂((ethoxycarbonyl)cyclopropyl), —O(CH₂)₀₋₂(oxetanyl), —O(CH₂)₀₋₂(methylazetidinyl), —O(CH₂)₁₋₂(morpholinyl), —O(CH₂)₀₋₂(tetrahydropyranyl), —O(CH₂)₀₋₂(thiazolyl), cyclopropyl, cyanocyclopropyl, methylazetidinyl, acetylazetidinyl, (tert-butoxycarbonyl)azetidinyl, dioxolanyl, pyrrolidinonyl, triazolyl, tetrahydropyranyl, morpholinyl, thiophenyl, methylpiperidinyl, and R_{d}; or
   (iii) C₁₋₃ alkyl substituted with one cyclic group selected from C₃₋₆ cycloalkyl, 4- to 10-membered heterocyclyl, mono- or bicyclic aryl, or 5- to 10-membered heteroaryl, said cyclic group substituted with zero to 3 substituents independently selected from F, Cl, Br, —OH, —CN, C₁₋₃ alkyl, C₁₋₂ fluoroalkyl, C₁₋₃ alkoxy, C₁₋₂ fluoroalkoxy, —OCH₂CH═CH₂, —OCH₂C═CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₄ cycloalkyl;
   or R₄ₐ and R_{4b} together with the carbon atom to which they are attached, form a C₃₋₆ cycloalkyl or a 3- to 6-membered heterocyclyl, each substituted with zero to 3 R_{f};
   each R_{f} is independently F, Cl, Br, —OH, —CN, C₁₋₄ alkyl, C₁₋₂ fluoroalkyl, C₁₋₃ alkoxy, C₁₋₂ fluoroalkoxy, —OCH₂CH═CH₂, —OCH₂C═CH, —NR_{c}R_{c}, or a cyclic group selected from C₃₋₆ cycloalkyl, 3- to 6-membered heterocyclyl, phenyl, monocyclic heteroaryl, and bicyclic heteroaryl, each cyclic group substituted with zero to 3 substituents independently selected from F, Cl, Br, —OH, —CN, C₁₋₄ alkyl, C₁₋₂ fluoroalkyl, C₁₋₃ alkoxy, C₁₋₂ fluoroalkoxy, and —NR_{c}R_{c};
   R_{4c} is C₁₋₄ alkyl or C₃₋₆ cycloalkyl, each substituted with zero to 4 substituents independently selected from F, Cl, —OH, C₁₋₂ alkoxy, C₁₋₂ fluoroalkoxy, and —CN;
   each R₅ is independently —CN, C₁₋₅ alkyl substituted with zero to 4 R_{g}, C₂₋₃ alkenyl substituted with zero to 4 R_{g}, C₂₋₃ alkynyl substituted with zero to 4 R_{g}, C₃₋₄ cycloalkyl substituted with zero to 4 R_{g}, phenyl substituted with zero to 3 R_{g}, oxadiazolyl substituted with zero to 3 R_{g}, pyridinyl substituted with zero to 3 R_{g}, —(CH₂)₁₋₂ (4- to 10-membered heterocyclyl substituted with zero to 4 R_{g}), —(CH₂)₁₋₂NR_{c}C(O)(C₁₋₄ alkyl), —(CH₂)₁₋₂NR_{c}C(O)O(C₁₋₄ alkyl), —(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl);
each Rₓ is independently H or —CH₃; and
m is 1, 2, or 3.

A method of treating a disease, such as cancer, may comprise administering to a subject in need thereof an antagonist of the PD1/PD-L1 axis and/or an antagonist of CTLA4 and an inhibitor of DGKα and/or DGKζ that is a compound of Formula (II) or a pharmaceutically acceptable salt thereof wherein m is 2; one R₅ is R₅ₐ and the other R₅ is R_{5c}; and said compound has the structure of Formula (III):

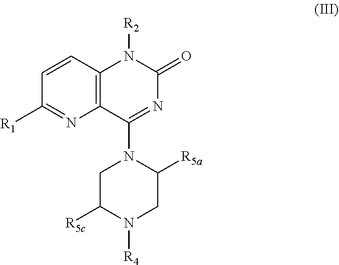

R₅ₐ is —CH₃ or —CH₂CH₃; and
R_{5c} is —CH₃, —CH₂CH₃, or —CH₂CH₂CH₃.

A method of treating a disease, such as cancer, may comprise administering to a subject in need thereof an antagonist of the PD1/PD-L1 axis and/or an antagonist of CTLA4 and an inhibitor of DGKα and/or DGKζ that is a compound of Formula (III) or a pharmaceutically acceptable salt thereof wherein

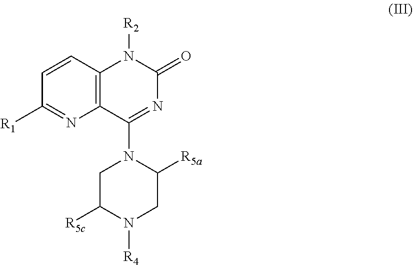

### R₁ is —CN;

R₂ is —CH₃;
R₅ₐ is —CH₃ or —CH₂CH₃; and
R_{5c} is —CH₃, —CH₂CH₃, or —CH₂CH₂CH₃.

A method of treating a disease, such as cancer, may comprise administering to a subject in need thereof an antagonist of the PD1/PD-L1 axis and/or an antagonist of CTLA4 and an inhibitor of DGKα and/or DGKζ that is a compound of Formula (II) or a pharmaceutically acceptable salt thereof having one the following structures:
4-((2S,5R)-2,5-diethyl-4-(1-(4-(trifluoromethyl)phenyl)propyl) piperazin-1-yl)-1-methyl-2-oxo-1,2-dihydropyrido[3,2-d]pyrimidine-6-carbonitrile

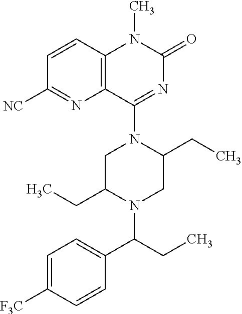 [SMILES: CCC1CN(C(CC)c2ccc(C(F)(F)F)cc2)C(CC)CN1c1nc(=O)n(C)c2ccc(C#N)nc12]

including 4-((2S,5R)-2,5-diethyl-4-((S)-1-(4-(trifluoromethyl)phenyl) propyl)piperazin-1-yl)-1-methyl-2-oxo-1,2-dihydropyrido[3,2-d]pyrimidine-6-carbonitrile and 4-((2S,5R)-2,5-diethyl-4-((R)-1-(4-(trifluoromethyl)phenyl) propyl)piperazin-1-yl)-1-methyl-2-oxo-1,2-dihydropyrido[3,2-d]pyrimidine-6-carbonitrile;
4-((2S,5R)-5-ethyl-2-methyl-4-(1-(4-(trifluoromethyl)phenyl)ethyl)piperazin-1-yl)-1-methyl-2-oxo-1,2-dihydropyrido[3,2-d]pyrimidine-6-carbonitrile

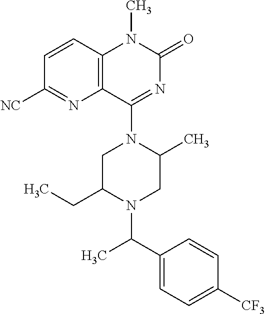 [SMILES: CCC1CN(c2nc(=O)n(C)c3ccc(C#N)nc23)C(C)CN1C(C)c1ccc(C(F)(F)F)cc1]

including 4-((2S,5R)-5-ethyl-2-methyl-4-((S)-1-(4-(trifluoromethyl)phenyl)ethyl) piperazin-1-yl)-1-methyl-2-oxo-1,2-dihydropyrido[3,2-d]pyrimidine-6-carbonitrile and 4-((2S,5R)-5-ethyl-2-methyl-4-((R)-1-(4-(trifluoromethyl)phenyl)ethyl)piperazin-1-yl)-1-methyl-2-oxo-1,2-dihydropyrido[3,2-d]pyrimidine-6-carbonitrile;
4-((2S,5R)-5-ethyl-4-((4-fluorophenyl)(5-(trifluoromethyl) pyridin-2-yl)methyl)-2-methylpiperazin-1-yl)-1-methyl-2-oxo-1,2-dihydropyrido[3,2-d]pyrimidine-6-carbonitrile

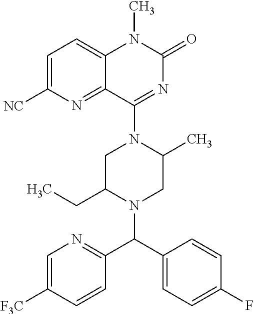 [SMILES: CCC1CN(c2nc(=O)n(C)c3ccc(C#N)nc23)C(C)CN1C(c1ccc(F)cc1)c1ccc(C(F)(F)F)cn1]

including 4-((2S,5R)-5-ethyl-4-((S)-(4-fluorophenyl)(5-(trifluoromethyl) pyridin-2-yl)methyl)-2-methylpiperazin-1-yl)-1-methyl-2-oxo-1,2-dihydropyrido[3,2-d]pyrimidine-6-carbonitrile and 4-((2S,5R)-5-ethyl-4-((R)-(4-fluorophenyl)(5-(trifluoromethyl) pyridin-2-yl)methyl)-2-methylpiperazin-1-yl)-1-methyl-2-oxo-1,2-dihydropyrido[3,2-d]pyrimidine-6-carbonitrile;
4-((2S,5R)-5-ethyl-2-methyl-4-(1-(4-(trifluoromethoxy)phenyl)ethyl)piperazin-1-yl)-1-methyl-2-oxo-1,2-dihydropyrido[3,2-d]pyrimidine-6-carbonitrile

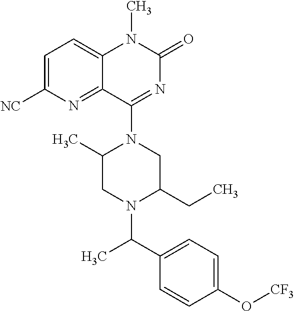 [SMILES: CCC1CN(c2nc(=O)n(C)c3ccc(C#N)nc23)C(C)CN1C(C)c1ccc(OC(F)(F)F)cc1]

including 4-((2S,5R)-5-ethyl-2-methyl-4-((S)-1-(4-(trifluoromethoxy)phenyl)ethyl) piperazin-1-yl)-1-methyl-2-oxo-1,2-dihydropyrido[3,2-d]pyrimidine-6-carbonitrile and 4-((2S,5R)-5-ethyl-2-methyl-4-((R)-1-(4-(trifluoromethoxy)phenyl)ethyl)piperazin-1-yl)-1-methyl-2-oxo-1,2-dihydropyrido[3,2-d]pyrimidine-6-carbonitrile;
4-((2S,5R)-5-ethyl-2-methyl-4-(1-(4-(trifluoromethoxy)phenyl)propyl)piperazin-1-yl)-1-methyl-2-oxo-1,2-dihydropyrido[3,2-d]pyrimidine-6-carbonitrile

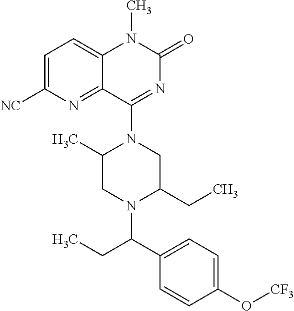 [SMILES: CCC1CN(c2nc(=O)n(C)c3ccc(C#N)nc23)C(C)CN1C(CC)c1ccc(OC(F)(F)F)cc1]

including 4-((2S,5R)-5-ethyl-2-methyl-4-((S)-1-(4-(trifluoromethoxy)phenyl)propyl) piperazin-1-yl)-1-methyl-2-oxo-1,2-dihydropyrido[3,2-d]pyrimidine-6-carbonitrile and 4-((2S,5R)-5-ethyl-2-methyl-4-((R)-1-(4-(trifluoromethoxy)phenyl)propyl)piperazin-1-yl)-1-methyl-2-oxo-1,2-dihydropyrido[3,2-d]pyrimidine-6-carbonitrile;
4-((2S,5R)-5-ethyl-2-methyl-4-(1-(4-(trifluoromethyl)phenyl) propyl)piperazin-1-yl)-1-methyl-2-oxo-1,2-dihydropyrido[3,2-d]pyrimidine-6-carbonitrile

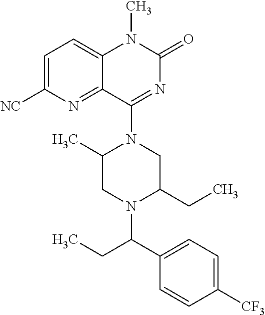 [SMILES: CCC1CN(c2nc(=O)n(C)c3ccc(C#N)nc23)C(C)CN1C(CC)c1ccc(C(F)(F)F)cc1]

including 4-((2S,5R)-5-ethyl-2-methyl-4-((S)-1-(4-(trifluoromethyl)phenyl)propyl) piperazin-1-yl)-1-methyl-2-oxo-1,2-dihydropyrido[3,2-d]pyrimidine-6-carbonitrile and 4-((2S,5R)-5-ethyl-2-methyl-4-((R)-1-(4-(trifluoromethyl)phenyl)propyl)piperazin-1-yl)-1-methyl-2-oxo-1,2-dihydropyrido[3,2-d]pyrimidine-6-carbonitrile;
4-((2S,5R)-4-((4-chlorophenyl)(pyridin-2-yl)methyl)-5-ethyl-2-methylpiperazin-1-yl)-1-methyl-2-oxo-1,2-dihydropyrido[3,2-d]pyrimidine-6-carbonitrile

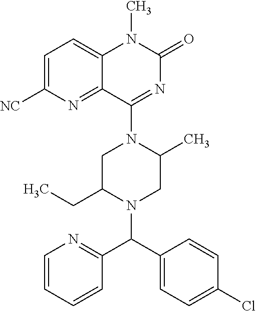 [SMILES: CCC1CN(c2nc(=O)n(C)c3ccc(C#N)nc23)C(C)CN1C(c1ccc(Cl)cc1)c1ccccn1]

including 4-((2S,5R)-4-((R)-(4-chlorophenyl)(pyridin-2-yl)methyl)-5-ethyl-2-methylpiperazin-1-yl)-1-methyl-2-oxo-1,2-dihydropyrido[3,2-d]pyrimidine-6-carbonitrile and 4-((2S,5R)-4-((S)-(4-chlorophenyl)(pyridin-2-yl)methyl)-5-ethyl-2-methylpiperazin-1-yl)-1-methyl-2-oxo-1,2-dihydropyrido[3,2-d]pyrimidine-6-carbonitrile;
4-((2S,5R)-4-((3-cyclopropyl-1,2,4-oxadiazol-5-yl)(4-fluorophenyl)methyl)-2,5-dimethylpiperazin-1-yl)-1-methyl-2-oxo-1,2-dihydropyrido[3,2-d]pyrimidine-6-carbonitrile

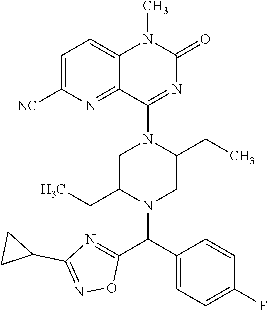 [SMILES: CCC1CN(C(c2ccc(F)cc2)c2nc(C3CC3)no2)C(CC)CN1c1nc(=O)n(C)c2ccc(C#N)nc12]

including 4-((2S,5R)-4-((R)-(3-cyclopropyl-1,2,4-oxadiazol-5-yl)(4-fluorophenyl)methyl)-2,5-diethylpiperazin-1-yl)-1-methyl-2-oxo-1,2-dihydropyrido[3,2-d]pyrimidine-6-carbonitrile and 4-((2S,5R)-4-((S)-(3-cyclopropyl-1,2,4-oxadiazol-5-yl)(4-fluorophenyl)methyl)-2,5-diethylpiperazin-1-yl)-1-methyl-2-oxo-1,2-dihydropyrido[3,2-d]pyrimidine-6-carbonitrile;
4-((2S,5R)-4-((4-fluorophenyl)(5-(trifluoromethyl)pyridin-2-yl)methyl)-2,5-dimethylpiperazin-1-yl)-1-methyl-2-oxo-1,2-dihydropyrido[3,2-d]pyrimidine-6-carbonitrile

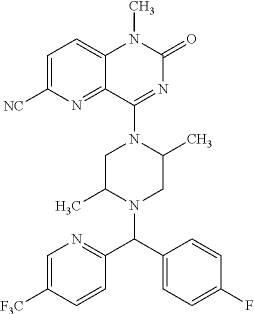 [SMILES: CC1CN(C(c2ccc(F)cc2)c2ccc(C(F)(F)F)cn2)C(C)CN1c1nc(=O)n(C)c2ccc(C#N)nc12]

including 4-((2S,5R)-4-((S)-(4-fluorophenyl)(5-(trifluoromethyl)pyridin-2-yl)methyl)-2,5-dimethylpiperazin-1-yl)-1-methyl-2-oxo-1,2-dihydropyrido[3,2-d]pyrimidine-6-carbonitrile and 4-((2S,5R)-4-((R)-(4-fluorophenyl)(5-(trifluoromethyl)pyridin-2-yl)methyl)-2,5-dimethylpiperazin-1-yl)-1-methyl-2-oxo-1,2-dihydropyrido[3,2-d]pyrimidine-6-carbonitrile;
4-((2S,5R)-4-(1-(4-(cyclopropylmethoxy)-2-fluorophenyl) propyl)-2,5-diethylpiperazin-1-yl)-1-methyl-2-oxo-1,2-dihydropyrido[3,2-d]pyrimidine-6-carbonitrile

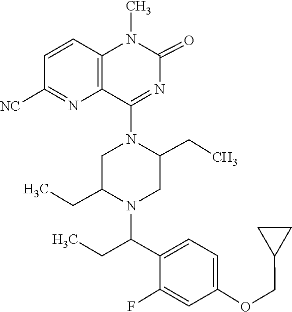 [SMILES: CCC1CN(C(CC)c2ccc(OCC3CC3)cc2F)C(CC)CN1c1nc(=O)n(C)c2ccc(C#N)nc12]

including 4-((2S,5R)-4-((S)-1-(4-(cyclopropylmethoxy)-2-fluorophenyl) propyl)-2,5-diethylpiperazin-1-yl)-1-methyl-2-oxo-1,2-dihydropyrido[3,2-d]pyrimidine-6-carbonitrile and 4-((2S,5R)-4-((R)-1-(4-(cyclopropylmethoxy)-2-fluorophenyl) propyl)-2,5-diethylpiperazin-1-yl)-1-methyl-2-oxo-1,2-dihydropyrido[3,2-d]pyrimidine-6-carbonitrile;
4-((2S,5R)-2,5-diethyl-4-(1-(4-(trifluoromethyl)phenyl)butyl)piperazin-1-yl)-1-methyl-2-oxo-1,2-dihydropyrido[3,2-d]pyrimidine-6-carbonitrile

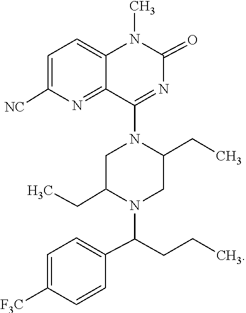 [SMILES: CCCC(c1ccc(C(F)(F)F)cc1)N1CC(CC)N(c2nc(=O)n(C)c3ccc(C#N)nc23)CC1CC]

including 4-((2S,5R)-2,5-diethyl-4-((S)-1-(4-(trifluoromethyl)phenyl)butyl)piperazin-1-yl)-1-methyl-2-oxo-1,2-dihydropyrido[3,2-d]pyrimidine-6-carbonitrile and 4-((2S,5R)-2,5-diethyl-4-((R)-1-(4-(trifluoromethyl)phenyl)butyl)piperazin-1-yl)-1-methyl-2-oxo-1,2-dihydropyrido[3,2-d]pyrimidine-6-carbonitrile;
1-methyl-4-((2S,5R)-2-methyl-5-propyl-4-(1-(4-(trifluoromethyl)phenyl)ethyl)piperazin-1-yl)-2-oxo-1,2-dihydropyrido[3,2-d]pyrimidine-6-carbonitrile

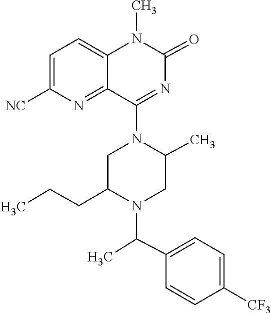 [SMILES: CCCC1CN(c2nc(=O)n(C)c3ccc(C#N)nc23)C(C)CN1C(C)c1ccc(C(F)(F)F)cc1]

including 1-methyl-4-((2S,5R)-2-methyl-5-propyl-4-((S)-1-(4-(trifluoromethyl)phenyl)ethyl)piperazin-1-yl)-2-oxo-1,2-dihydropyrido[3,2-d]pyrimidine-6-carbonitrile and 1-methyl-4-((2S,5R)-2-methyl-5-propyl-4-((R)-1-(4-(trifluoromethyl)phenyl)ethyl) piperazin-1-yl)-2-oxo-1,2-dihydropyrido[3,2-d]pyrimidine-6-carbonitrile.

Antagonists of the PD1/PD-L1 Axis

Antagonists of the PD1/PD-L1 axis that can be combined with DGK inhibitors include the following.

An antagonist of the PD1/PD-L1 axis is an antagonist of human PD1 or an antagonist of human PD-L1 that stimulates an immune response by inhibiting a negative checkpoint. An antagonist may be any type of molecule, e.g., a protein, nucleic acid or a small molecule. In certain embodiments, an antagonist of the PD1/PD-L1 axis is an antibody that binds specifically to human PD1 or human PD-L1.

Anti-PD-1 antibodies that are known in the art can be used in the presently described methods. Various human monoclonal antibodies that bind specifically to PD-1 with high affinity have been disclosed in U.S. Pat. No. 8,008,449. Anti-PD-1 human antibodies disclosed in U.S. Pat. No. 8,008,449 have been demonstrated to exhibit one or more of the following characteristics: (a) bind to human PD-1 with a KD of 1×10-⁷ M or less, as determined by surface plasmon resonance using a Biacore biosensor system; (b) do not substantially bind to human CD28, CTLA-4 or ICOS; (c) increase T-cell proliferation in a Mixed Lymphocyte Reaction (MLR) assay; (d) increase interferon-γ production in an MLR assay; (e) increase IL-2 secretion in an MLR assay; (f) bind to human PD-1 and cynomolgus monkey PD-1; (g) inhibit the binding of PD-L1 and/or PD-L2 to PD-1; (h) stimulate antigen-specific memory responses; (i) stimulate antibody responses; and (j) inhibit tumor cell growth in vivo. Anti-PD-1 antibodies usable in the present disclosure include monoclonal antibodies that bind specifically to human PD-1 and exhibit at least one, in some aspects, at least five, of the preceding characteristics.

Other anti-PD-1 monoclonal antibodies have been described in, for example, U.S. Pat. Nos. 6,808,710, 7,488,802, 8,168,757 and 8,354,509, US Publication No. 2016/0272708, and PCT Publication Nos. WO 2012/145493, WO 2008/156712, WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/133540, WO 2017/132827, WO 2017/024465, WO 2017/025016, WO 2017/106061, WO 2017/19846, WO 2017/024465, WO 2017/025016, WO 2017/132825, and WO 2017/133540 each of which is incorporated by reference in its entirety.

In some aspects, the anti-PD-1 antibody is selected from the group consisting of nivolumab (also known as OPDIVO®, 5C4, BMS-936558, MDX-1106, and ONO-4538), pembrolizumab (Merck; also known as KEYTRUDA®, lambrolizumab, and MK-3475; see WO2008/156712), PDR001 (Novartis; see WO 2015/112900), MEDI-0680 (AstraZeneca; also known as AMP-514; see WO 2012/145493), cemiplimab (Regeneron; also known as REGN-2810; see WO 2015/112800), JS001 (TAIZHOU JUNSHI PHARMA; also known as toripalimab; see Si-Yang Liu et al., J. Hematol. Oncol. 10:136 (2017)), sintilimab, BGB-A317 (Beigene; also known as Tislelizumab; see WO 2015/35606 and US 2015/0079109), INCSHRI210 (Jiangsu Hengrui Medicine; also known as SHR-1210; see WO 2015/085847; Si-Yang Liu et al., J. Hematol. Oncol. 10:136 (2017)), TSR-042 (Tesaro Biopharmaceutical; also known as ANBO11; see WO2014/179664), GLS-010 (Wuxi/Harbin Gloria Pharmaceuticals; also known as WBP3055; see Si-Yang Liu et al., J. Hematol. Oncol. 10:136 (2017)), AM-0001 (Armo), STI-1110 (Sorrento Therapeutics; see WO 2014/194302), AGEN2034 (Agenus; see WO 2017/040790), MGA012 (Macrogenics, see WO 2017/19846), BCD-100 (Biocad; Kaplon et al., mAbs 10(2):183-203 (2018), and IBI308 (Innovent; see WO 2017/024465, WO 2017/025016, WO 2017/132825, and WO 2017/133540).

In one aspect, the anti-PD-1 antibody is nivolumab. Nivolumab is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56).

In another aspect, the anti-PD-1 antibody is pembrolizumab. Pembrolizumab is a humanized monoclonal IgG4 (S228P) antibody directed against human cell surface receptor PD-1 (programmed death-1 or programmed cell death-1). Pembrolizumab is described, for example, in U.S. Pat. Nos. 8,354,509 and 8,900,587.

Anti-PD-1 antibodies usable in the disclosed methods also include isolated antibodies that bind specifically to human PD-1 and cross-compete for binding to human PD-1 with any anti-PD-1 antibody disclosed herein, e.g., nivolumab (see, e.g., U.S. Pat. Nos. 8,008,449 and 8,779,105; WO 2013/173223). In some aspects, the anti-PD-1 antibody binds the same epitope as any of the anti-PD-1 antibodies described herein, e.g., nivolumab. The ability of antibodies to cross-compete for binding to an antigen indicates that these monoclonal antibodies bind to the same epitope region of the antigen and sterically hinder the binding of other cross-competing antibodies to that particular epitope region. These cross-competing antibodies are expected to have functional properties very similar those of the reference antibody, e.g., nivolumab, by virtue of their binding to the same epitope region of PD-1. Cross-competing antibodies can be readily identified based on their ability to cross-compete with nivolumab in standard PD-1 binding assays such as Biacore analysis, ELISA assays or flow cytometry (see, e.g., WO 2013/173223).

In certain aspects, the antibodies that cross-compete for binding to human PD-1 with, or bind to the same epitope region of human PD-1 antibody, nivolumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-1 antibodies usable in the methods of the disclosed disclosure also include antigen-binding portions of the above antibodies. It has been amply demonstrated that the antigen-binding function of an antibody can be performed by fragments of a full-length antibody.

Anti-PD-1 antibodies suitable for use in the disclosed compositions and methods are antibodies that bind to PD-1 with high specificity and affinity, block the binding of PD-L1 and or PD-L2, and inhibit the immunosuppressive effect of the PD-1 signaling pathway. In any of the compositions or methods disclosed herein, an anti-PD-1 “antibody” includes an antigen-binding portion or fragment that binds to the PD-1 receptor and exhibits the functional properties similar to those of whole antibodies in inhibiting ligand binding and up-regulating the immune system. In certain aspects, the anti-PD-1 antibody or antigen-binding portion thereof cross-competes with nivolumab for binding to human PD-1.

In certain aspects, an antagonist of the PD1/PD-L1 axis is an antagonist of PD-L1. Anti-PD-L1 antibodies that are known in the art can be used in the compositions and methods of the present disclosure. Examples of anti-PD-L1 antibodies useful in the compositions and methods of the present disclosure include the antibodies disclosed in U.S. Pat. No. 9,580,507. Anti-PD-L1 human monoclonal antibodies disclosed in U.S. Pat. No. 9,580,507 have been demonstrated to exhibit one or more of the following characteristics: (a) bind to human PD-L1 with a KD of 1×10-⁷ M or less, as determined by surface plasmon resonance using a Biacore biosensor system; (b) increase T-cell proliferation in a Mixed Lymphocyte Reaction (MLR) assay; (c) increase interferon-γ production in an MLR assay; (d) increase IL-2 secretion in an MLR assay; (e) stimulate antibody responses; and (f) reverse the effect of T regulatory cells on T cell effector cells and/or dendritic cells. Anti-PD-L1 antibodies usable in the present disclosure include monoclonal antibodies that bind specifically to human PD-L1 and exhibit at least one, in some aspects, at least five, of the preceding characteristics.

In certain aspects, the anti-PD-L1 antibody is selected from the group consisting of BMS-936559 (also known as 12A4, MDX-1105; see, e.g., U.S. Pat. No. 7,943,743 and WO 2013/173223), atezolizumab (Roche; also known as TECENTRIQ®; MPDL3280A, RG7446; see U.S. Pat. No. 8,217,149; see, also, Herbst et al. (2013) J Clin Oncol 31(suppl):3000), durvalumab (AstraZeneca; also known as IMFINZI™, MEDI-4736; see WO 2011/066389), avelumab (Pfizer; also known as BAVENCIO®, MSB-0010718C; see WO 2013/079174), STI-1014 (Sorrento; see WO2013/181634), CX-072 (Cytomx; see WO2016/149201), KN035 (3D Med/Alphamab; see Zhang et al., Cell Discov. 7:3 (March 2017), LY3300054 (Eli Lilly Co.; see, e.g., WO 2017/034916), BGB-A333 (BeiGene; see Desai et al., JCO 36 (15suppl): TPS3113 (2018)), and CK-301 (Checkpoint Therapeutics; see Gorelik et al., AACR:Abstract 4606 (April 2016)).

In certain aspects, the PD-L1 antibody is atezolizumab (TECENTRIQ®). Atezolizumab is a fully humanized IgG1 monoclonal anti-PD-L1 antibody.

In certain aspects, the PD-L1 antibody is durvalumab (IMFINZI™). Durvalumab is a human IgG1 kappa monoclonal anti-PD-L1 antibody.

In certain aspects, the PD-L1 antibody is avelumab (BAVENCIO™). Avelumab is a human IgG1 lambda monoclonal anti-PD-L1 antibody.

Anti-PD-L1 antibodies usable in the disclosed methods also include isolated antibodies that bind specifically to human PD-L1 and cross-compete for binding to human PD-L1 with any anti-PD-L1 antibody disclosed herein, e.g., atezolizumab, durvalumab, and/or avelumab. In some aspects, the anti-PD-L1 antibody binds the same epitope as any of the anti-PD-L1 antibodies described herein, e.g., atezolizumab, durvalumab, and/or avelumab. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of the reference antibody, e.g., atezolizumab and/or avelumab, by virtue of their binding to the same epitope region of PD-L1. Cross-competing antibodies can be readily identified based on their ability to cross-compete with atezolizumab and/or avelumab in standard PD-L1 binding assays such as Biacore analysis, ELISA assays or flow cytometry (see, e.g., WO 2013/173223).

In certain aspects, the antibodies that cross-compete for binding to human PD-L1 with, or bind to the same epitope region of human PD-L1 antibody as, atezolizumab, durvalumab, and/or avelumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-L1 antibodies usable in the methods of the disclosed disclosure also include antigen-binding portions of the above antibodies. It has been amply demonstrated that the antigen-binding function of an antibody can be performed by fragments of a full-length antibody.

Anti-PD-L1 antibodies suitable for use in the disclosed methods are antibodies that bind to PD-L1 with high specificity and affinity, block the binding of PD-1, and inhibit the immunosuppressive effect of the PD-1 signaling pathway. In any of the methods disclosed herein, an anti-PD-L1 “antibody” includes an antigen-binding portion or fragment that binds to PD-L1 and exhibits the functional properties similar to those of whole antibodies in inhibiting receptor binding and up-regulating the immune system. In certain aspects, the anti-PD-L1 antibody or antigen-binding portion thereof cross-competes with atezolizumab, durvalumab, and/or avelumab for binding to human PD-L1.

The anti-PD-L1 antibody useful for the present disclosure can be any PD-L1 antibody that specifically binds to PD-L1, e.g., antibodies that cross-compete with durvalumab, avelumab, or atezolizumab for binding to human PD-1, e.g., an antibody that binds to the same epitope as durvalumab, avelumab, or atezolizumab. In a particular aspect, the anti-PD-L1 antibody is durvalumab. In other aspects, the anti-PD-L1 antibody is avelumab. In some aspects, the anti-PD-L1 antibody is atezolizumab.

Antagonists of CTLA4

Antagonists of CTLA4 that can be combined with DGK inhibitors include the following.

An antagonist of CTLA-4 is an antagonist of human CTLA-4 that stimulates an immune response by inhibiting a negative checkpoint. An antagonist may be any type of molecule, e.g., a protein, nucleic acid or a small molecule. In certain embodiments, an antagonist of CTLA-4 is an antibody that binds specifically to human CTLA-4.

Anti-CTLA-4 antibodies that are known in the art can be used in the methods of the present disclosure. Anti-CTLA-4 antibodies of the instant disclosure bind to human CTLA-4 so as to disrupt the interaction of CTLA-4 with a human B7 receptor. Because the interaction of CTLA-4 with B7 transduces a signal leading to inactivation of T-cells bearing the CTLA-4 receptor, disruption of the interaction effectively induces, enhances or prolongs the activation of such T cells, thereby inducing, enhancing or prolonging an immune response.

Human monoclonal antibodies that bind specifically to CTLA-4 with high affinity have been disclosed in U.S. Pat. No. 6,984,720. Other anti-CTLA-4 monoclonal antibodies have been described in, for example, U.S. Pat. Nos. 5,977,318, 6,051,227, 6,682,736, and 7,034,121 and International Publication Nos. WO 2012/122444, WO 2007/113648, WO 2016/196237, and WO 2000/037504, each of which is incorporated by reference herein in its entirety. The anti-CTLA-4 human monoclonal antibodies disclosed in U.S. Pat. No. 6,984,720 have been demonstrated to exhibit one or more of the following characteristics: (a) binds specifically to human CTLA-4 with a binding affinity reflected by an equilibrium association constant (Ka) of at least about 107 M−1, or about 109 M−1, or about 1010 M−1 to 1011 M−1 or higher, as determined by Biacore analysis; (b) a kinetic association constant (ka) of at least about 103, about 104, or about 105 m−1 s−1; (c) a kinetic disassociation constant (kd) of at least about 103, about 104, or about 105 m−1 s−1; and (d) inhibits the binding of CTLA-4 to B7-1 (CD80) and B7-2 (CD86). Anti-CTLA-4 antibodies useful for the present disclosure include monoclonal antibodies that bind specifically to human CTLA-4 and exhibit at least one, at least two, or at least three of the preceding characteristics.

In certain aspects, the CTLA-4 antibody is selected from the group consisting of ipilimumab (also known as YERVOY®, MDX-010, 1ODI; see U.S. Pat. No. 6,984,720), MK-1308 (Merck), AGEN-1884 (Agenus Inc.; see WO 2016/196237), and tremelimumab (AstraZeneca; also known as ticilimumab, CP-675,206; see WO 2000/037504 and Ribas, Update Cancer Ther. 2(3): 133-39 (2007)). In particular aspects, the anti-CTLA-4 antibody is ipilimumab.

In particular aspects, the CTLA-4 antibody is ipilimumab for use in the methods disclosed herein. Ipilimumab is a fully human, IgG1 monoclonal antibody that blocks the binding of CTLA-4 to its B7 ligands, thereby stimulating T cell activation and improving overall survival (OS) in patients with advanced melanoma.

In particular aspects, the CTLA-4 antibody is tremelimumab.

In particular aspects, the CTLA-4 antibody is MK-1308.

In particular aspects, the CTLA-4 antibody is AGEN-1884.

Anti-CTLA-4 antibodies usable in the disclosed methods also include isolated antibodies that bind specifically to human CTLA-4 and cross-compete for binding to human CTLA-4 with any anti-CTLA-4 antibody disclosed herein, e.g., ipilimumab and/or tremelimumab. In some aspects, the anti-CTLA-4 antibody binds the same epitope as any of the anti-CTLA-4 antibodies described herein, e.g., ipilimumab and/or tremelimumab. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of the reference antibody, e.g., ipilimumab and/or tremelimumab, by virtue of their binding to the same epitope region of CTLA-4. Cross-competing antibodies can be readily identified based on their ability to cross-compete with ipilimumab and/or tremelimumab in standard CTLA-4 binding assays such as Biacore analysis, ELISA assays or flow cytometry (see, e.g., WO 2013/173223).

In certain aspects, the antibodies that cross-compete for binding to human CTLA-4 with, or bind to the same epitope region of human CTLA-4 antibody as, ipilimumab and/or tremelimumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-CTLA-4 antibodies usable in the methods of the disclosed disclosure also include antigen-binding portions of the above antibodies. It has been amply demonstrated that the antigen-binding function of an antibody can be performed by fragments of a full-length antibody.

Anti-CTLA-4 antibodies suitable for use in the disclosed methods are antibodies that bind to CTLA-4 with high specificity and affinity, block the activity of CTLA-4, and disrupt the interaction of CTLA-4 with a human B7 receptor. In any of the compositions or methods disclosed herein, an anti-CTLA-4 “antibody” includes an antigen-binding portion or fragment that binds to CTLA-4 and exhibits the functional properties similar to those of whole antibodies in inhibiting the interaction of CTLA-4 with a human B7 receptor and up-regulating the immune system. In certain aspects, the anti-CTLA-4 antibody or antigen-binding portion thereof cross-competes with ipilimumab and/or tremelimumab for binding to human CTLA-4.

Antagonists of CTLA4 also include variants of CTLA4 antibodies. Exemplary variants of CTLA4 antibodies are non-fucosylated anti-CTLA4 antibodies, such as non-fucosylated ipilimumab, activatable CTLA4 antibodies having a mask that is selectively cleaved within tumors, such as activatable ipilimumab, or activatable CTLA-4 antibodies that are non-fucosylated. Exemplary non-fucosylated and/or activatable anti-CTLA4 antibodies, e.g., ipilimumab, are provided in WO2014/089113 and WO2018/085555.

Administration of Inhibitors of DGKα and/or DGKζ and Antagonists of the PD1/PD-L1 Axis or CTLA4

Compounds described herein, e.g., in accordance with Formula (I) or (II), such as a compound selected from compounds 1 to 34, and/or pharmaceutically acceptable salts thereof, can be administered by any means suitable for the condition to be treated, which can depend on the need for site-specific treatment or quantity of the compound to be delivered.

Also embraced herein is a class of pharmaceutical compositions comprising a compound, e.g., a compound of Formula (I) or (II), such as a compound selected from compounds 1 to 34, and/or a pharmaceutically acceptable salt thereof; and one or more non-toxic, pharmaceutically-acceptable carriers and/or diluents and/or adjuvants (collectively referred to herein as “carrier” materials) and, if desired, other active ingredients. The compounds, e.g., the compounds of Formula (I) or (II), such as a compound selected from compounds 1 to 34, may be administered by any suitable route, preferably in the form of a pharmaceutical composition adapted to such a route, and in a dose effective for the treatment intended. The compounds and compositions described herein may, for example, be administered orally, mucosally, or parentally including intravascularly, intravenously, intraperitoneally, subcutaneously, intramuscularly, and intrasternally in dosage unit formulations containing conventional pharmaceutically acceptable carriers, adjuvants, and vehicles. For example, the pharmaceutical carrier may contain a mixture of mannitol or lactose and microcrystalline cellulose. The mixture may contain additional components such as a lubricating agent, e.g. magnesium stearate and a disintegrating agent such as crospovidone. The carrier mixture may be filled into a gelatin capsule or compressed as a tablet. The pharmaceutical composition may be administered as an oral dosage form or an infusion, for example.

For oral administration, a pharmaceutical composition described herein may be in the form of, for example, a tablet, capsule, liquid capsule, suspension, or liquid. The pharmaceutical composition is preferably made in the form of a dosage unit containing a particular amount of the active ingredient. For example, the pharmaceutical composition may be provided as a tablet or capsule comprising an amount of active ingredient in the range of from about 0.1 to 1000 mg, preferably from about 0.25 to 250 mg, and more preferably from about 0.5 to 100 mg. A suitable daily dose for a human or other mammal may vary widely depending on the condition of the patient and other factors, but, can be determined using routine methods.

Any pharmaceutical composition contemplated herein can, for example, be delivered orally via any acceptable and suitable oral preparations. Exemplary oral preparations, include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition can contain at least one agent selected from sweetening agents, flavoring agents, coloring agents, demulcents, antioxidants, and preserving agents.

A tablet can, for example, be prepared by admixing at least one compound, e.g., a compound of Formula (I) or (II), such as a compound selected from compounds 1 to 34, and/or at least one pharmaceutically acceptable salt thereof, with at least one non-toxic pharmaceutically acceptable excipient suitable for the manufacture of tablets. Exemplary excipients include, but are not limited to, for example, inert diluents, such as, for example, calcium carbonate, sodium carbonate, lactose, calcium phosphate, and sodium phosphate; granulating and disintegrating agents, such as, for example, microcrystalline cellulose, sodium crosscarmellose, corn starch, and alginic acid; binding agents, such as, for example, starch, gelatin, polyvinyl-pyrrolidone, and acacia; and lubricating agents, such as, for example, magnesium stearate, stearic acid, and talc. Additionally, a tablet can either be uncoated, or coated by known techniques to either mask the bad taste of an unpleasant tasting drug, or delay disintegration and absorption of the active ingredient in the gastrointestinal tract thereby sustaining the effects of the active ingredient for a longer period. Exemplary water soluble taste masking materials, include, but are not limited to, hydroxypropyl-methylcellulose and hydroxypropyl-cellulose. Exemplary time delay materials, include, but are not limited to, ethyl cellulose and cellulose acetate butyrate.

Hard gelatin capsules can, for example, be prepared by mixing at least one compound, e.g., a compound of Formula (I) or (II), such as a compound selected from compounds 1 to 34, and/or at least one pharmaceutically acceptable salt thereof, with at least one inert solid diluent, such as, for example, calcium carbonate; calcium phosphate; and kaolin.

Soft gelatin capsules can, for example, be prepared by mixing at least one compound, e.g., a compound of Formula (I) or (II), such as a compound selected from compounds 1 to 34 and/or at least one pharmaceutically acceptable salt thereof, with at least one water soluble carrier, such as, for example, polyethylene glycol; and at least one oil medium, such as, for example, peanut oil, liquid paraffin, and olive oil.

An aqueous suspension can be prepared, for example, by admixing at least one compound, e.g., a compound of Formula (I) or (II), such as a compound selected from compounds 1 to 34, and/or at least one pharmaceutically acceptable salt thereof, with at least one excipient suitable for the manufacture of an aqueous suspension. Exemplary excipients suitable for the manufacture of an aqueous suspension, include, but are not limited to, for example, suspending agents, such as, for example, sodium carboxymethylcellulose, methylcellulose, hydroxypropylmethyl-cellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long chain aliphatic alcohols, such as, for example heptadecaethylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Oily suspensions can, for example, be prepared by suspending at least one compound, e.g., a compound of Formula (I) or (II), such as a compound selected from compounds 1 to 34, and/or at least one pharmaceutically acceptable salt thereof, in either a vegetable oil, such as, for example, arachis oil; olive oil; sesame oil; and coconut oil; or in mineral oil, such as, for example, liquid paraffin. An oily suspension can also contain at least one thickening agent, such as, for example, beeswax; hard paraffin; and cetyl alcohol. In order to provide a palatable oily suspension, at least one of the sweetening agents already described hereinabove, and/or at least one flavoring agent can be added to the oily suspension. An oily suspension can further contain at least one preservative, including, but not limited to, for example, an anti-oxidant, such as, for example, butylated hydroxyanisol, and alpha-tocopherol.

Dispersible powders and granules can, for example, be prepared by admixing at least one compound, e.g., a compound of Formula (I) or (II), such as a compound selected from compounds 1 to 34, and/or at least one pharmaceutically acceptable salt thereof, with at least one dispersing and/or wetting agent; at least one suspending agent; and/or at least one preservative. Suitable dispersing agents, wetting agents, and suspending agents are as already described above. Exemplary preservatives include, but are not limited to, for example, anti-oxidants, e.g., ascorbic acid. In addition, dispersible powders and granules can also contain at least one excipient, including, but not limited to, for example, sweetening agents; flavoring agents; and coloring agents.

An emulsion of at least one compound, e.g., a compound of Formula (I) or (II), such as a compound selected from compounds 1 to 34, and/or at least one pharmaceutically acceptable salt thereof, can, for example, be prepared as an oil-in-water emulsion. The oily phase of the emulsions comprising compounds of Formula (I) or (II), such as a compound selected from compounds 1 to 34 may be constituted from known ingredients in a known manner. The oil phase can be provided by, but is not limited to, for example, a vegetable oil, such as, for example, olive oil and arachis oil; a mineral oil, such as, for example, liquid paraffin; and mixtures thereof. While the phase may comprise merely an emulsifier, it may comprise a mixture of at least one emulsifier with a fat or an oil or with both a fat and an oil. Suitable emulsifying agents include, but are not limited to, for example, naturally-occurring phosphatides, e.g., soy bean lecithin; esters or partial esters derived from fatty acids and hexitol anhydrides, such as, for example, sorbitan monooleate; and condensation products of partial esters with ethylene oxide, such as, for example, polyoxyethylene sorbitan monooleate. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. An emulsion can also contain a sweetening agent, a flavoring agent, a preservative, and/or an antioxidant. Emulsifiers and emulsion stabilizers suitable for use in the formulation for use in the treatment methods include Tween 60, Span 80, cetostearyl alcohol, myristyl alcohol, glyceryl monostearate, sodium lauryl sulfate, glyceryl distearate alone or with a wax, or other materials well known in the art.

The compounds, e.g., those of Formula (I) or (II), such as a compound selected from compounds 1 to 34, and/or at least one pharmaceutically acceptable salt thereof, can, for example, also be delivered intravenously, subcutaneously, and/or intramuscularly via any pharmaceutically acceptable and suitable injectable form. Exemplary injectable forms include, but are not limited to, for example, sterile aqueous solutions comprising acceptable vehicles and solvents, such as, for example, water, Ringer's solution, and isotonic sodium chloride solution; sterile oil-in-water microemulsions; and aqueous or oleaginous suspensions.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers including saline, dextrose, or water, or with cyclodextrin (i.e. Captisol), cosolvent solubilization (i.e. propylene glycol) or micellar solubilization (i.e. Tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

A sterile injectable oil-in-water microemulsion can, for example, be prepared by 1) dissolving at least one compound, e.g., a compound of Formula (I) or (II), such as a compound selected from compounds 1 to 34, and/or a pharmaceutically acceptable salt thereof, in an oily phase, such as, for example, a mixture of soybean oil and lecithin; 2) combining a compound, e.g., a compound of Formula (I), and/or a pharmaceutically acceptable salt thereof, containing oil phase with a water and glycerol mixture; and 3) processing the combination to form a microemulsion.

A sterile aqueous or oleaginous suspension can be prepared in accordance with methods already known in the art. For example, a sterile aqueous solution or suspension can be prepared with a non-toxic parenterally-acceptable diluent or solvent, such as, for example, 1,3-butane diol; and a sterile oleaginous suspension can be prepared with a sterile non-toxic acceptable solvent or suspending medium, such as, for example, sterile fixed oils, e.g., synthetic mono- or diglycerides; and fatty acids, such as, for example, oleic acid.

Pharmaceutically acceptable carriers, adjuvants, and vehicles that may be used in the pharmaceutical compositions include, but are not limited to, ion exchangers, alumina, aluminum stearate, lecithin, self-emulsifying drug delivery systems (SEDDS) such as d-alpha-tocopherol polyethyleneglycol 1000 succinate, surfactants used in pharmaceutical dosage forms such as Tweens, polyethoxylated castor oil such as CREMOPHOR surfactant (BASF), or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as alpha-, beta-, and gamma-cyclodextrin, or chemically modified derivatives such as hydroxyalkylcyclodextrins, including 2- and 3-hydroxypropyl-cyclodextrins, or other solubilized derivatives may also be advantageously used to enhance delivery of compounds of the formulae described herein.

The pharmaceutically active compounds described herein can be processed in accordance with conventional methods of pharmacy to produce medicinal agents for administration to patients, including humans and other mammals. The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such compositions may also comprise adjuvants, such as wetting, sweetening, flavoring, and perfuming agents.

The amounts of compounds that are administered and the dosage regimen for treating a disease condition with the compounds and/or compositions described herein depends on a variety of factors, including the age, weight, sex, the medical condition of the subject, the type of disease, the severity of the disease, the route and frequency of administration, and the particular compound employed. Thus, the dosage regimen may vary widely, but can be determined routinely using standard methods. A daily dose of about 0.001 to 100 mg/kg body weight, preferably between about 0.0025 and about 50 mg/kg body weight and most preferably between about 0.005 to 10 mg/kg body weight, may be appropriate. The daily dose can be administered in one to four doses per day. Other dosing schedules include one dose per week and one dose per two day cycle.

For therapeutic purposes, the active compounds described herein are ordinarily combined with one or more adjuvants appropriate to the indicated route of administration. If administered orally, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose.

Pharmaceutical compositions described herein comprise at least one compound, e.g., a compound of Formula (I), and/or at least one pharmaceutically acceptable salt thereof, and optionally an additional agent selected from any pharmaceutically acceptable carrier, adjuvant, and vehicle. Alternate compositions described herein comprise a compound, such as a compound of the Formula (I) or (II), such as a compound selected from compounds 1 to 34 described herein, or a prodrug thereof, and a pharmaceutically acceptable carrier, adjuvant, or vehicle.

In some aspects, an anti-PD-L1 antibody used in the treatment methods described herein is administered at a dose ranging from about 0.1 mg/kg to about 20.0 mg/kg body weight, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg, about once every 2, 3, 4, 5, 6, 7, or 8 weeks.

In some aspects, the anti-PD-L1 antibody is administered at a dose of about 15 mg/kg body weight at about once every 3 weeks. In other aspects, the anti-PD-L1 antibody is administered at a dose of about 10 mg/kg body weight at about once every 2 weeks.

In other aspects, the anti-PD-L1 antibody useful for the present disclosure is a flat dose. In some aspects, the anti-PD-L1 antibody is administered as a flat dose of from about 200 mg to about 1600 mg, about 200 mg to about 1500 mg, about 200 mg to about 1400 mg, about 200 mg to about 1300 mg, about 200 mg to about 1200 mg, about 200 mg to about 1100 mg, about 200 mg to about 1000 mg, about 200 mg to about 900 mg, about 200 mg to about 800 mg, about 200 mg to about 700 mg, about 200 mg to about 600 mg, about 700 mg to about 1300 mg, about 800 mg to about 1200 mg, about 700 mg to about 900 mg, or about 1100 mg to about 1300 mg. In some aspects, the anti-PD-L1 antibody is administered as a flat dose of at least about 240 mg, at least about 300 mg, at least about 320 mg, at least about 400 mg, at least about 480 mg, at least about 500 mg, at least about 560 mg, at least about 600 mg, at least about 640 mg, at least about 700 mg, at least 720 mg, at least about 800 mg, at least about 840 mg, at least about 880 mg, at least about 900 mg, at least 960 mg, at least about 1000 mg, at least about 1040 mg, at least about 1100 mg, at least about 1120 mg, at least about 1200 mg, at least about 1280 mg, at least about 1300 mg, at least about 1360 mg, or at least about 1400 mg, at a dosing interval of about 1, 2, 3, or 4 weeks. In some aspects, the anti-PD-L1 antibody is administered as a flat dose of about 1200 mg at about once every 3 weeks. In other aspects, the anti-PD-L1 antibody is administered as a flat dose of about 800 mg at about once every 2 weeks. In other aspects, the anti-PD-L1 antibody is administered as a flat dose of about 840 mg at about once every 2 weeks.

In some aspects, atezolizumab is administered as a flat dose of about 1200 mg once about every 3 weeks. In some aspects, atezolizumab is administered as a flat dose of about 800 mg once about every 2 weeks. In some aspects, atezolizumab is administered as a flat dose of about 840 mg once about every 2 weeks.

In some aspects, avelumab is administered as a flat dose of about 800 mg once about every 2 weeks.

In some aspects, durvalumab is administered at a dose of about 10 mg/kg once about every 2 weeks. In some aspects, durvalumab is administered as a flat dose of about 800 mg/kg once about every 2 weeks. In some aspects, durvalumab is administered as a flat dose of about 1200 mg/kg once about every 3 weeks.

In some aspects, the anti-CTLA-4 antibody or antigen-binding portion thereof used in the treatment methods described herein is administered at a dose ranging from 0.1 mg/kg to 10.0 mg/kg body weight once every 2, 3, 4, 5, 6, 7, or 8 weeks. In some aspects, the anti-CTLA-4 antibody or antigen-binding portion thereof is administered at a dose of 1 mg/kg or 3 mg/kg body weight once every 3, 4, 5, or 6 weeks. In one aspect, the anti-CTLA-4 antibody or antigen-binding portion thereof is administered at a dose of 3 mg/kg body weight once every 2 weeks. In another aspect, the anti-PD-1 antibody or antigen-binding portion thereof is administered at a dose of 1 mg/kg body weight once every 6 weeks.

In some aspects, the anti-CTLA-4 antibody or antigen-binding portion thereof is administered as a flat dose. In some aspects, the anti-CTLA-4 antibody is administered at a flat dose of from about 10 to about 1000 mg, from about 10 mg to about 900 mg, from about 10 mg to about 800 mg, from about 10 mg to about 700 mg, from about 10 mg to about 600 mg, from about 10 mg to about 500 mg, from about 100 mg to about 1000 mg, from about 100 mg to about 900 mg, from about 100 mg to about 800 mg, from about 100 mg to about 700 mg, from about 100 mg to about 100 mg, from about 100 mg to about 500 mg, from about 100 mg to about 480 mg, or from about 240 mg to about 480 mg. In one aspect, the anti-CTLA-4 antibody or antigen-binding portion thereof is administered as a flat dose of at least about 60 mg, at least about 80 mg, at least about 100 mg, at least about 120 mg, at least about 140 mg, at least about 160 mg, at least about 180 mg, at least about 200 mg, at least about 220 mg, at least about 240 mg, at least about 260 mg, at least about 280 mg, at least about 300 mg, at least about 320 mg, at least about 340 mg, at least about 360 mg, at least about 380 mg, at least about 400 mg, at least about 420 mg, at least about 440 mg, at least about 460 mg, at least about 480 mg, at least about 500 mg, at least about 520 mg at least about 540 mg, at least about 550 mg, at least about 560 mg, at least about 580 mg, at least about 600 mg, at least about 620 mg, at least about 640 mg, at least about 660 mg, at least about 680 mg, at least about 700 mg, or at least about 720 mg. In another aspect, the anti-CTLA-4 antibody or antigen-binding portion thereof is administered as a flat dose about once every 1, 2, 3, 4, 5, 6, 7, or 8 weeks.

In some aspects, ipilimumab is administered at a dose of about 3 mg/kg once about every 3 weeks. In some aspects, ipilimumab is administered at a dose of about 10 mg/kg once about every 3 weeks. In some aspects, ipilimumab is administered at a dose of about 10 mg/kg once about every 12 weeks. In some aspects, the ipilimumab is administered for four doses.

### Methods of Preparation of Compounds

The compounds described herein may be synthesized by many methods available to those skilled in the art of organic chemistry. General synthetic schemes for preparing encompassed herein are described below. These schemes are illustrative and are not meant to limit the possible techniques one skilled in the art may use to prepare the compounds disclosed herein. Different methods to prepare the compounds encompassed herein will be evident to those skilled in the art. Examples of compounds prepared by methods described in the general schemes are given in the Examples section set out hereinafter. Preparation of homochiral examples may be carried out by techniques known to one skilled in the art. For example, homochiral compounds may be prepared by separation of racemic products or diastereomers by chiral phase preparative HPLC. Alternatively, the example compounds may be prepared by methods known to give enantiomerically or diastereomerically enriched products.

The reactions and techniques described in this section are performed in solvents appropriate to the reagents and materials employed and are suitable for the transformations being effected. Also, in the description of the synthetic methods given below, it is to be understood that all proposed reaction conditions, including choice of solvent, reaction atmosphere, reaction temperature, duration of the experiment and work up procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents that are compatible with the reaction conditions will be readily apparent to one skilled in the art, with alternatives required when incompatible substituents are present. This will sometimes require a judgment to modify the order of the synthetic steps or to select one particular process scheme over another in order to obtain a desired compound. It will also be recognized that another major consideration in the planning of any synthetic route in this field is the judicious choice of a protecting group used for protection of reactive functional groups present in the compounds described herein. An authoritative account describing the many alternatives to the trained practitioner is Wuts and Greene, Greene's Protective Groups in Organic Synthesis, Fourth Edition, Wiley and Sons (2007).

### EXAMPLES

The following examples illustrate the particular and preferred embodiments of the present disclosure and do not limit the scope of the present disclosure. Chemical abbreviations and symbols as well as scientific abbreviations and symbols have their usual and customary meanings unless otherwise specified. Additional abbreviations employed in the Examples and elsewhere in this application are defined herein. Common intermediates are generally useful for the preparation of more than one Example and are identified sequentially (e.g., Intermediate 1, Intermediate 2, etc.) and are abbreviated as Int. 1 or I1, Int. 2 or 12, etc. In some instances alternate preparations of intermediates or examples are described. Frequently chemists skilled in the art of synthesis may devise alternative preparations which may be desirable based on one or more considerations such as shorter reaction time, less expensive starting materials, ease of operation or isolation, improved yield, amenable to catalysis, avoidance of toxic reagents, accessibility of specialized instrumentation, and decreased number of linear steps, etc. The intent of describing alternative preparations is to further enable the preparation of the examples of this disclosure. In some instances some functional groups in the outlined examples and claims may be replaced by well-known bioisosteric replacements known in the art, for example, replacement of a carboxylic acid group with a tetrazole or a phosphate moiety. ¹H NMR data collected in deuterated dimethyl sulfoxide used water suppression in the data processing. The reported spectra are uncorrected for the effects of water suppression. Protons adjacent to the water suppression frequency of 3.35 ppm exhibit diminished signal intensity.

### Abbreviations

Ac acetyl
anhyd. anhydrous
aq. aqueous
aza-HOBt 7-aza-1-hydroxybenzotriazole
Bn benzyl
1-BOC-piperazine tert-butyl piperazine-1-carboxylate
Bu butyl
CV Column Volumes
DCE dichloroethane
DCM dichloromethane
DEA diethylamine
DIEA diisopropyl ethyl amine (Hunig's base)
DIPEA diisopropyl ethyl amine
DMA N,N-dimethylacetamide
DMF dimethylformamide
DMSO dimethyl sulfoxide
EA ethyl acetate
EDC 1-(3-dimethylaminopropyl)-3-ethylcarbodiimide hydrochloride
Et ethyl
h, hours or hrs hour(s)
HATU (1-[bis(dimethylamino)methylene]-1H-1,2,3-triazolo[4,5-b]pyridinium 3-oxid hexafluorophosphate)
HCl hydrochloric acid
HPLC high pressure liquid chromatography
KHMDS potassium bis(trimethylsilyl)amide
LC liquid chromatography
LCMS liquid chromatography-mass spectrometry
M molar
mM millimolar
Me methyl
MHz megahertz
mins minute(s)
M+1 (M+H)+
MS mass spectrometry
n or N normal
NaHMDS sodium bis(trimethylsilyl)amide
NBS N-bromosuccinimide
nM nanomolar
NMP N-methylpyrrolidinone
Ph phenyl
PYBROP bromotripyrrolidinophosphonium hexafluorophosphate
RuPhos precatalyst chloro(2-dicyclohexylphosphino-2′,6′-diisopropoxy-1,1′-biphenyl)[2-(2′-amino-1,1′-biphenyl)]palladium(II)
RT or Ret time retention time
sat. saturated
t-BuOH tertiary butanol
TEA triethylamine
TFA trifluoroacetic acid
THF tetrahydrofuran
TLC thin layer chromatography
POCl₃ phosphorous oxychloride
2^{nd} Gen Xphos CAS number 1310584-14-5

### Example 1: DGKi Enhances Activity of Nivolumab and Ipilimumab in the Alloreactive MLR Assay

This Example shows that inhibition of DGK enhances the activity of a PD-1 and a CTLA-4 inhibitor, as demonstrated by an increased level of interferon-γ (IFN-γ) secreted in an MLR assay.

The assay was conducted as follows. Peripheral blood mononuclear cells were isolated from EDTA treated whole blood using Ficoll cell separation. Cells were isolated further to T cells using a Stemcell EasySep human T cell enrichment kit (Stemcell 19051). Previously purchased frozen monocytes were allowed to thaw and were differentiated into dentritic cells (DCs) for six days with treatment of GMCSF and IL-4 in a 37° C. CO₂ incubator. T Cells were plated at 100 thousand cells per well into a 96 well round bottom plate in 10% FBS RPMI media. Allogeneic dendritic cells were added to the appropriate wells at a 10:1 ratio of T cells: immature DCs. The DGK inhibitor DGKi Compound 15 was diluted in DMSO and then further diluted into 10% FBS RPMI media and was added to the appropriate wells of T cell: immature DCs for a final DMSO concentration of 0.1% in a final volume of 250 μl. The mixed lymphocyte reaction was placed in the incubator for 5 days. On day 5, 130 μl of media was removed and 10 μl was used in an IFN-γ ELISA assay (BD cat 555142).

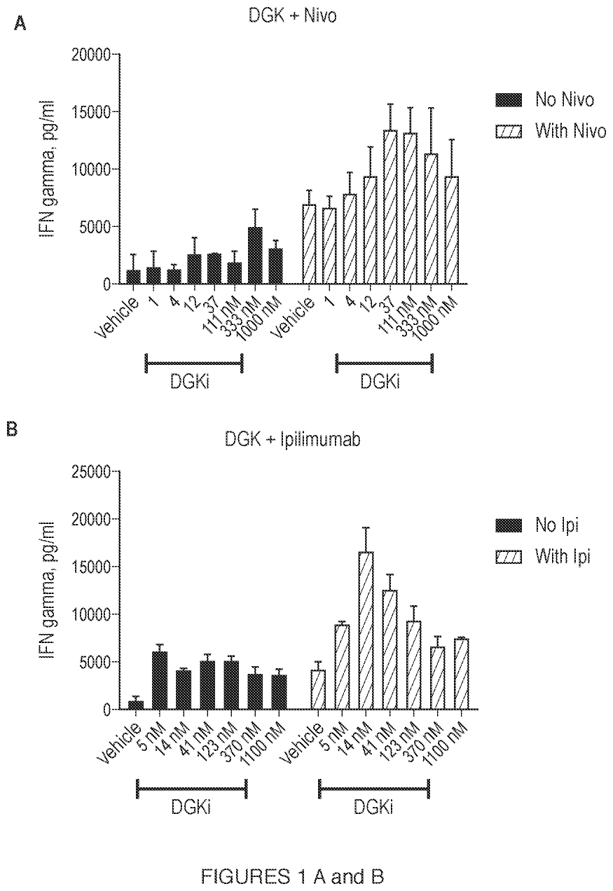

The results, which are shown in FIGS. 1A and B, indicate that inhibition of DGK enhances levels of IFN-γ secretion from T cells treated with a PD-1 or a CTLA-4 inhibitor.

### Example 2: Inhibition of DGK Enhances the Combined Activity of a PD-1 Antagonist and a CTLA4 Antagonist in the B16 Animal Tumor Model

This Example shows that administration of a DGKi at the same time as a PD-1 antagonist and a CTLA4 antagonist results in enhanced tumor reduction activity relative to the combination of the PD-1 antagonist and the CTLA4 antagonist.

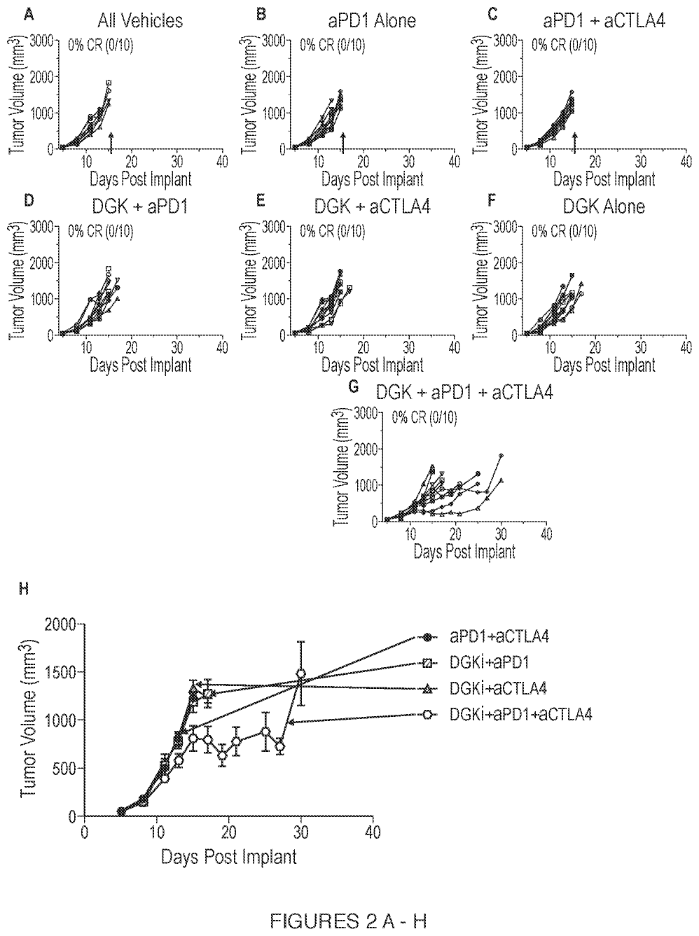

This assay was conducted in a B16 tumor model (a human melanoma tumor model). Mice were administered an anti-PD-1 antibody (mIgG1-D265A monoclonal antibody directed against mouse PD-1), an anti-CTLA4 antibody (mIgG2b monoclonal antibody directed against mouse CTLA4), vehicle alone, and/or a DGKi, and tumor growth was measured. The results, which are shown in FIGS. 2 A-G, indicate that no significant tumor reduction was observed with the individual agents or the combination of two agents, but that, combining the DGKi with the anti-PD-1 antibody and the anti-CTLA4 antibody resulted in tumor reduction (FIG. 2G).

### Example 3: Inhibition of DGK Enhances the Activity of a PD-1 Inhibitor and/or a CTLA-4 Inhibitor in the CT26 Animal Tumor Model

This Example shows that in the CT26 model, administration of a DGK inhibitor enhances tumor reduction induced by an anti-PD-1 and/or anti-CTLA4 antibody.

The assay was conducted as follows: CT26 cells (murine colorectal carcinoma cell line from the ATCC) were cultured in 10% fetal bovine serum (Invitrogen/ThermoFisher Scientific) and RPMI 1640 Medium (Gibco/ThermoFisher Scientific). Female BALB/c mice, obtained from Envigo arrived at 6-8 weeks of age. For tumor implantation (Day 0), mice were given a 0.1 mL subcutaneous injection of CT26 cell suspension at 1×107 cells/mL into the right flank. When tumors grew to a predetermined volume of ˜100 mm³, typically around 10 days post-implant, mice were randomized and sorted into various control and treatment groups and dosing was initiated. DGKi Compound 16 was formulated in 90% PEG400, 5% Ethanol, and 5% TPGS and given orally at a volume of 10 mL/kg body weight. Anti-CTLA4 (anti-mCTLA4, mIgG2b) and anti-PD1 (mIgG1-D265A monoclonal antibody directed against mouse PD-1) and isotype controls were diluted with DPBS to a dose of 10 mg/kg. Antibody therapies were administered via intraperitoneal injection (I.P.), every 4 days for a total of 3 doses (Q4Dx3). Tumor volumes were measured twice a week with a digital caliper until tumors had completely regressed (0 mm³) or reached 1000 mm³ and were euthanized. For AH1 tetramer staining, 100 μL of blood was collected from each mouse into a lithium heparin tube. Blood was stained with AH1 tetramer (MBL), anti-Cd3, anti-cd4, and anti-Cd8 (Biolegend). Samples were lysed using Lyse/Fix buffer (BD) and samples were acquired on a CantoX cytometer (BD), and analyzed in FlowJo (BD).

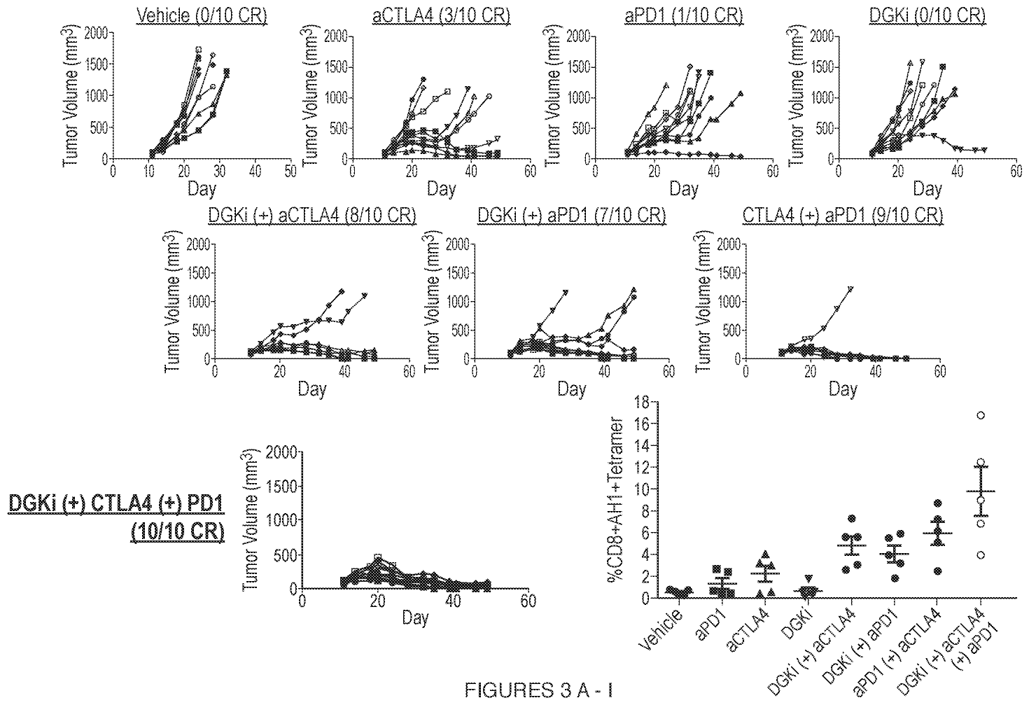

The results shown in FIGS. 3A-H, indicate that DGKi enhances tumor volume reduction induced by (i) a PD1 inhibitor; (ii) a CTLA-4 inhibitor; and (iii) a PD1 inhibitor and a CTLA-4 inhibitor, in the CT26 mouse model. The results shown in FIG. 31 indicate that DGKi increases the percentage of CD8 cells that are positive for AH1+Tetramer tumor antigen. Thus, the combination treatments result in improved complete responses and correlates with increased AH1+ T cells in the CT26 model. The combination of the DGK inhibitor with both a CTLA4 antagonist and a PD1 antagonist resulted in the highest number of complete responses, i.e., 10 out of 10 complete responses.

### Example 4: Inhibition of DGK Lowers the Antigen Threshold Required for TCR Activation

This Example shows that DGK inhibition (1) potentiates the T cell response induced by weak tumor antigens and (2) lowers the concentration of tumor antigen required for T cell activation.

This assay was conducted as follows: MC38 cells (murine colon adenocarcinoma cells) were acquired from and cultured in 10% fetal bovine serum (Invitrogen/ThermoFisher Scientific) and RPMI 1640 Medium (Gibco/ThermoFisher Scientific). Ova and heteroclitic peptide variants were acquired from AnaSpec and resuspended according to manufacturer's protocol. MC38 cells were pulsed with 1 μg/mL of peptide or indicated concentration for 3 hours then free peptide was washed out. OT1 mice (which are class I restricted TCR transgenic/C57B16 background with a TCR specific for ovalbumin (OVA (SIINFEKL) or the following derivatives of the OVA peptide: A2 (SAINFEKL), Q4 (SIIQFEKL), T4 (SIITFEKL), Q4H7 (SIIQFEHL), but does not recognize the scrambled peptide FILKSINE) were acquired from Jackson Labs. The TCR binding affinities of these peptides is shown in the Table below. CD8 T cells were purified from total splenocytes (StemCell) of the OT1 mice and activated using CD3/CD28 beads (Invitrogen) and then frozen. Frozen activated OT-1 CD8 T cells were thawed during the peptide pulse and plated with DGKi Compound 15 or control compound or DMSO for 1 hr. MC38-protein pulsed cells were added to the plate and co-cultured overnight at 37° C. Supernatants were collected and IL-2 was measured using AlphaLISA (PerkinElmer).

| Peptide | Sequence | K_{D} (μM) |
|---|---|---|
| OVA | SIINFEKL | 1 |
| A2 | SAINFEKL | 4 |
| Q4 | SIIQFEKL | 36 |
| T4 | SIITFEKL | 122 |
| Q4H7 | SIIQFEHL | 167 |
| Scrambled | FILKSINE | n.m. |

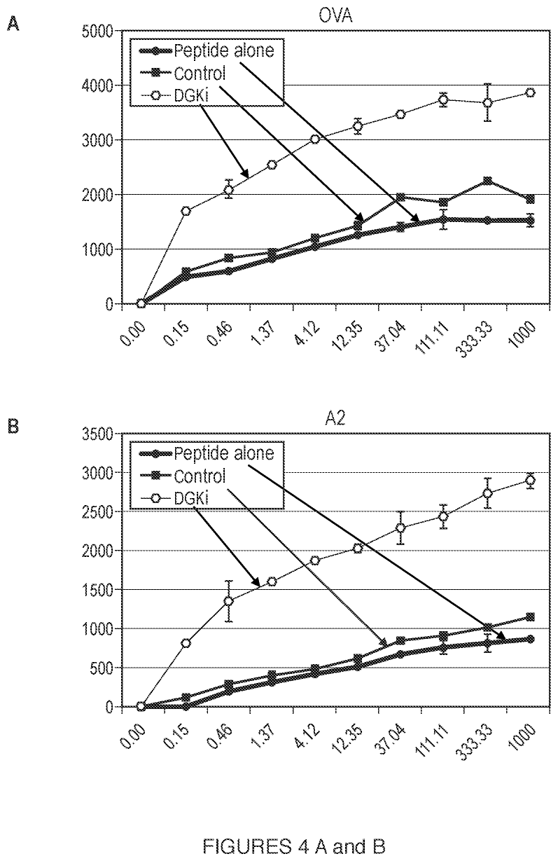

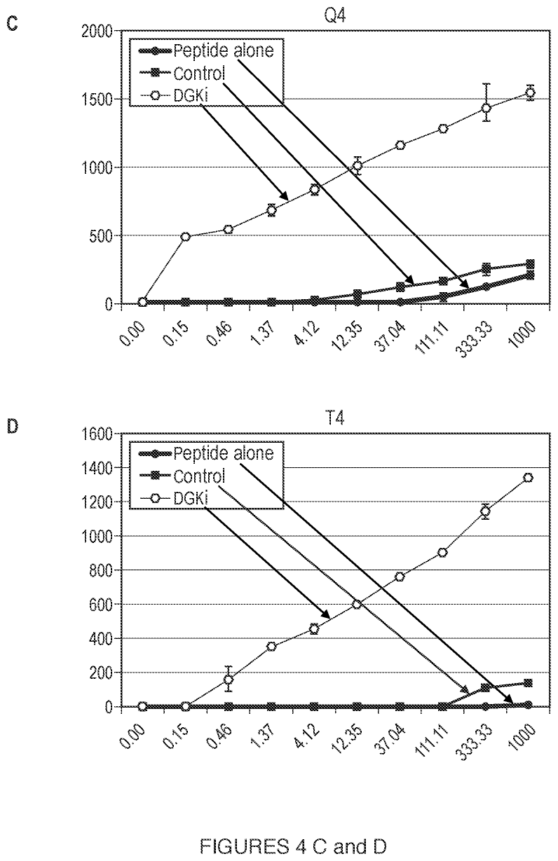

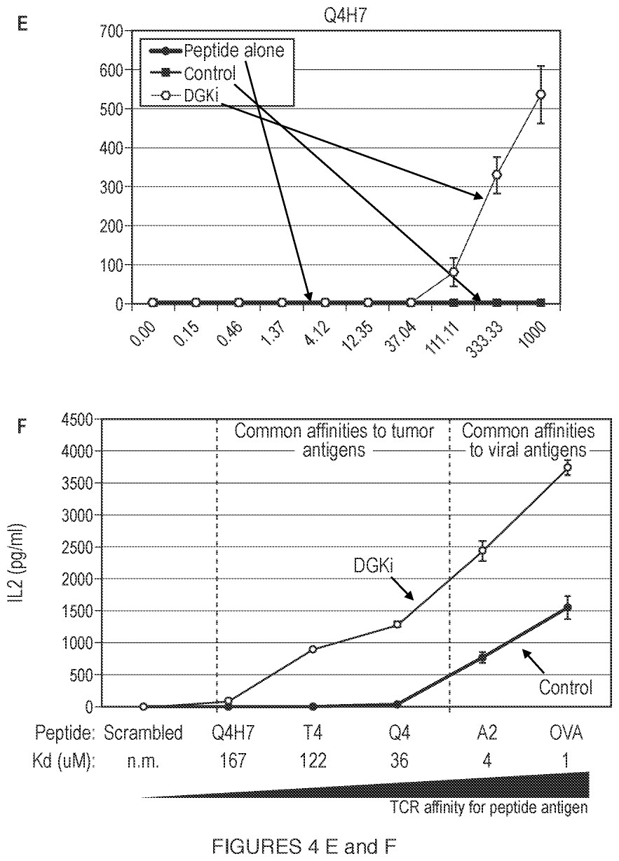

The results, which are shown in FIGS. 4A-F, indicate that the DGKi Compound 15 lowers both the affinity requirement and the concentration requirement of antigen for T cell antigen recognition and activation.

### Example 5: Inhibition of DGK Increases Human CTL Effector Function and Enhances Tumor Cell Killing

This Example shows that inhibition of DGK increases CTL effector function and tumor cell killing.

The assay was conducted as follows: HCT116-GFP (human colorectal cancer) cells were acquired from Cellomics. HCT116-GFP were pulsed with A2 and B35 peptides (Astarte) at indicated concentrations for 1 hour followed by washout. Cells were plated and allowed to adhere overnight. CMV specific human CD8 T cells (Astarte) were thawed, treated with DGKi Compound 15 for 1 hour then added to the HCT116-GFP cells. Supernatant was collected at 24 hours post co-culture and IFNg was measured using AlphaLISA (PerkinElmer). Images of GFP were taken using a fluorescent microscope.

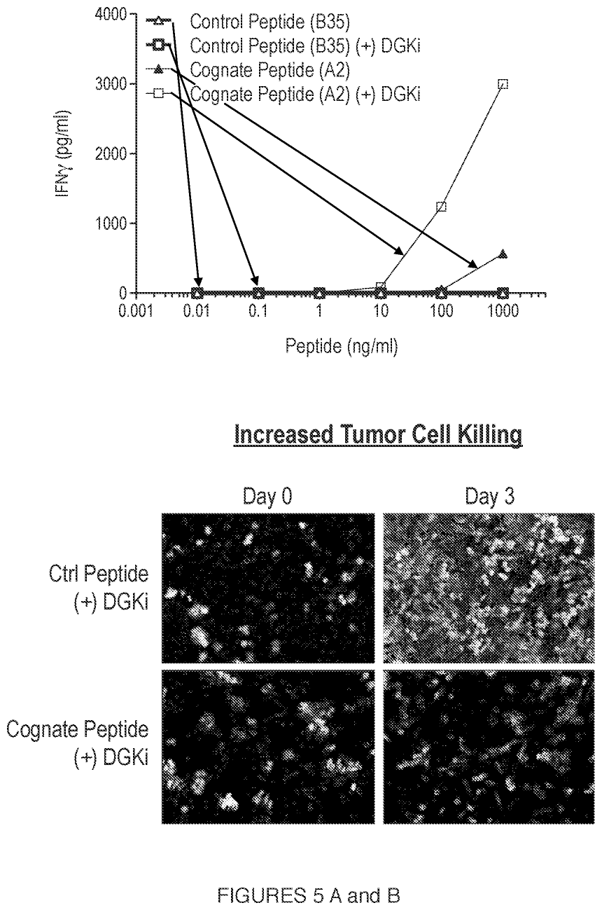

The results, which are shown in FIGS. 5 A and B show that the DGKi Compound 15 increases human CTL effector function and enhances tumor cell killing.

### Example 6: Inhibition of DGK can Overcome Decreased B2M Levels to Restore T Cell Effector Function

Many human tumors have mutations that result in partial or complete loss of class I MHC which is critical for T cells to recognize and kill tumor cells. This example shows that inhibition of DGK allows T cells to recognize tumor cells that have lower levels of MHC. These target cells would not otherwise be recognized by T cells.

The assay was conducted as follows: HCT116-GFP were acquired from Cellomics and cultured in 10% fetal bovine serum (Invitrogen/ThermoFisher Scientific) and RPMI 1640 Medium (Gibco/ThermoFisher Scientific). B2M guide RNA (Synthego) was introduced to HCT116-GFP cells by nucleofection (Lonza). After recovery, cells were plated in individual wells to generate single cell clones. Clones were stained for B2M (Biolegend) and evaluated by flow cytometry. Clones were then pulsed with A2 or B35 peptides (Astarte) at 1 mg/mL for 1 hour followed by washout. Cells were plated and allowed to adhere overnight. CMV specific human CD8 T cells (Astarte) were thawed, treated with DGKi Compound 15 for 1 hour and then added to the HCT116 cells. Supernatant was collected at 24 hours post co-culture and IFN-γ was measured using AlphaLISA (PerkinElmer).

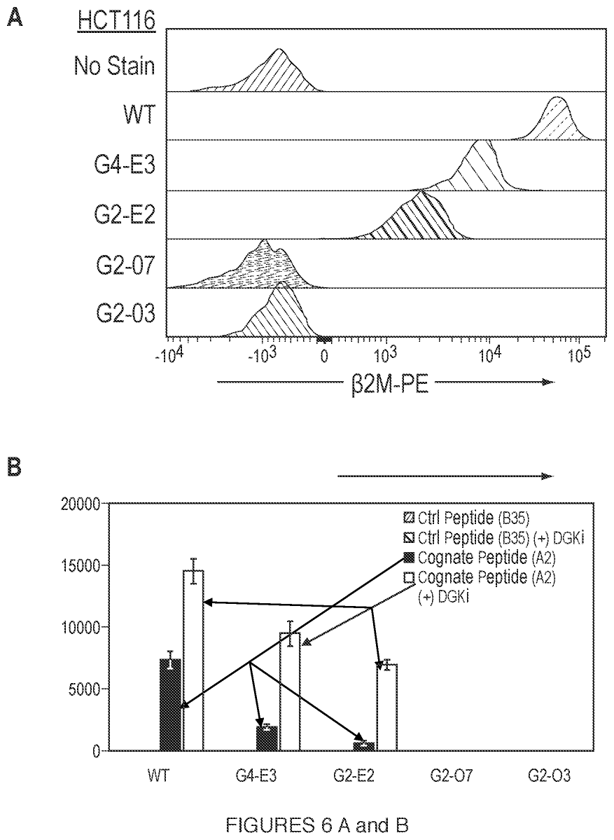

The results, which are shown in FIGS. 6 A and B, indicate that DGKi Compound 15 increases IFN-γ levels from T cells recognizing tumor cells having reduced class I MHC antigens.

### Example 7: Curative Tumor Activity by DGK Inhibition and a PD1 Antagonist is Dependent on CD8+ T Cells

This Example shows that curative tumor activity is dependent on CD8+ T cells in the CT26 animal model.

The assay was conducted as follows: CT26 cells (from the ATCC) were cultured in 10% fetal bovine serum (Invitrogen/ThermoFisher Scientific) and RPMI 1640 Medium (Gibco/ThermoFisher Scientific). Female BALB/c mice, obtained from Envigo arrived at 6-8 weeks of age. For tumor implantation (Day 0), mice were given a 0.1 mL subcutaneous injection of CT26 cell suspension at 1×10⁷ cells/mL into the right flank. CD8 depleting antibody (2.43, BioXCell) was diluted in PBS and dosed at 100 μg/mouse. Dosing was initiated on Day 1 and continued every 3-4 days until study completion. When tumors grew to a predetermined volume of ˜100 mm³, typically around 10 days post-implant, mice were randomized and sorted into various control and treatment groups and dosing was initiated. DGKi Compound 16 was formulated in 90% PEG400, 5% Ethanol, and 5% TPGS and given orally at a volume of 10 mL/kg body weight, dosed every 3 days for a total of 5 doses (Q3Dx5) at 5 mg/kg. Anti-PD1 antibody (mIgG1-D265A monoclonal antibody directed against mouse PD-1) and isotype control were diluted with DPBS to a dose of 10 mg/kg. Antibody therapies were administered via intraperitoneal injection (I.P.), every 4 days for a total of 3 doses (Q4Dx3). Tumor volumes were measured twice a week with a digital caliper until tumors had completely regressed (0 mm³) or reached 1000 mm³ and were euthanized.

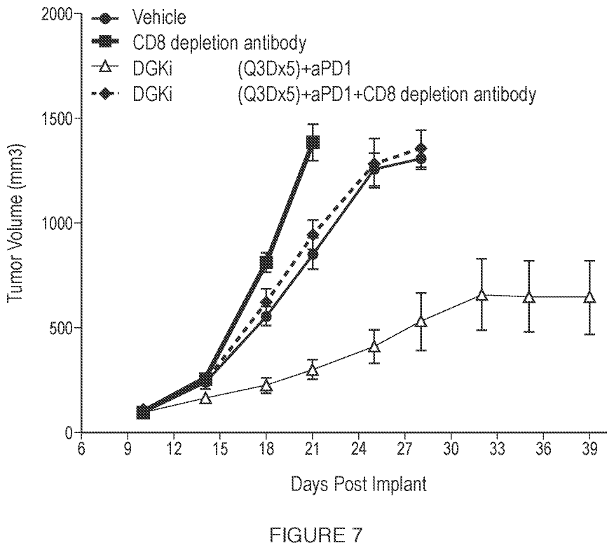

The results, which are shown in FIG. 7, indicate that the tumor volume reduction obtained by a treatment of CT26 mice with an anti-PD-1 antagonist and the DGKi Compound 16 is reduced by depletion of CD8+ cells.

### Example 8: Tumor Volume Reduction by DGK Inhibition and a PD1 Antagonist is Enhanced by CD4 Cell Depletion

This Example shows that the tumor reduction obtained by a combination of a DGK inhibitor and a PD-1 antagonist is further enhanced by the depletion of CD4 cells.

The assay was conducted as follows: CT26 cells (from the ATCC) were cultured in 10% fetal bovine serum (Invitrogen/ThermoFisher Scientific) and RPMI 1640 Medium (Gibco/ThermoFisher Scientific). Female BALB/c mice, obtained from Envigo arrived at 6-8 weeks of age. For tumor implantation (Day 0), mice were given a 0.1 mL subcutaneous injection of CT26 cell suspension at 1×10⁷ cells/mL into the right flank. CD4 depleting antibody (GK1.5, BioXCell) was diluted in PBS and dosed at 100 μg/mouse. Dosing was initiated on Day 1 and continued every 3-4 days until study completion. When tumors grew to a predetermined volume of ˜100 mm³, typically around 10 days post-implant, mice were randomized and sorted into various control and treatment groups and dosing was initiated. DGKi Compound 16 was formulated in 90% PEG400, 5% Ethanol, and 5% TPGS and given orally at a volume of 10 mL/kg body weight, every 3 days for a total of 5 doses (Q3Dx5) at 5 mg/kg. Anti-PD1 (mIgG1-D265A monoclonal antibody directed against mouse PD-1) and isotype control (MOPC-21, BioXCell) were diluted with DPBS to a dose of 10 mg/kg. Antibody therapies were administered via intraperitoneal injection (I.P.), every 4 days for a total of 3 doses (Q4Dx3). Tumor volumes were measured twice a week with a digital caliper until tumors had completely regressed (0 mm³) or reached 1000 mm³ and were euthanized.

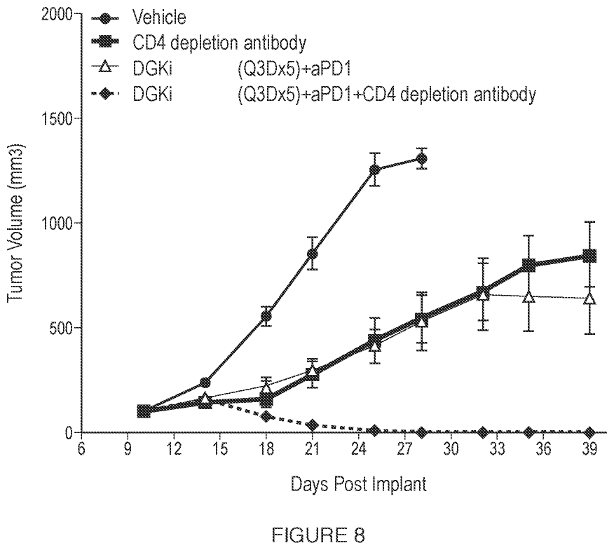

The results, which are shown in FIG. 8, indicate that the tumor volume reduction obtained by the treatment of MC38 mice with an anti-PD-1 antagonist and the DGKi Compound 16 is enhanced by depletion of CD4+ cells, presumably due to the depletion of Treg cells.

### Example 9: NK Cells are Required for DGKi and Anti-PD1 Anti-Tumor Efficacy

This Example shows that tumor reduction activity induced by a DGKi and a PD1 antagonist is dependent on NK cells in the CT26 animal model.

The assay was conducted essentially as described in Examples 6 and 7, but instead of adding an antibody binding to CD4 or CD8, anti-asialo-GM1 (Life Technologies) was dosed at 50 μg/mouse starting on D4 post tumor injection and continuing every 7 days until end of study.

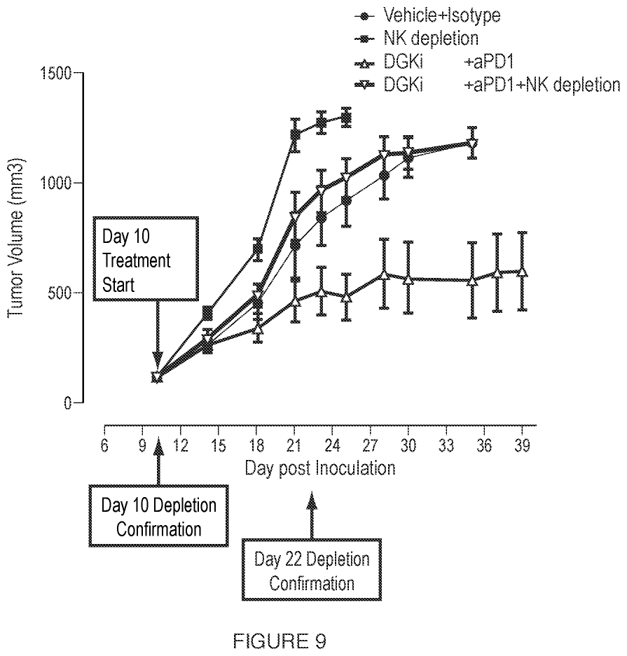

The results, which are shown in FIG. 9, indicate that NK cells contribute to the anti-tumor activity of the DGKi Compound 16 in combination with a PD1 inhibitor in the CT26 mouse model.

### Example 10: Combination of a DGKi of Formula II with Either Anti-PD-1 or Anti-CTLA4 Elicits Robust Efficacy

This Example shows that an exemplary DGKi of formula II from the group of compounds 17-34 together with anti-PD-1 or anti-CTLA4 antibody has strong anti-tumor activity in the MC38 animal model.

The assay was conducted as follows. The mouse colon adenocarcinoma tumor cell line MC38 was maintained in 10% fetal bovine serum (FBS, Invitrogen) and Roswell Park Memorial Institute (RPMI) 1640 Medium (Gibco) in T75 flasks. Cells were grown to subconfluency and passaged two times per week simply by rinsing with DPBS (Dulbecco's Phosphate-Buffered Saline, Gibco), allowing cells to sit for a few minutes and tapping the flask. MC38 cell passage ratios ranged from 1:16 to −1:20 depending on timing and confluency. For in-vivo implantation, cells were rinsed with DPBS and then collected in ice-cold HBSS (Hank's Balanced Salt Solution, Gibco) in 50 mL conical tubes on ice. Tubes were spun at 1300 rpm for 10 minutes, the supernatant carefully removed, and the pellets washed with HBSS and spun again. Pellets were resuspended in approximate implant volumes of HBSS. The cell concentration was measured using a Moxi-Z (Orflo) and adjusted to the final concentration with HBSS. Cell viability was measured using trypan blue exclusion on a Countess II (Life Technologies). Female C57Bl/6 mice, obtained from Charles River Laboratories (Kingston, N.Y.) were received in house at age 6-8 weeks and acclimated for 3-7 days. At the time of tumor implantation (day 0), mice were given a subcutaneous injection of 0.1 mL of MC38 cells at a concentration of 8.5×10⁶ cells/ml using a 1 mL tuberculin syringe with a 25 gauge needle, implanted into both the left and right flank. Tumors grew to a pre-determined size, ˜78 mm at which time animals were randomized into various treatment and control groups with like mean and median tumor values, where n=10 per group on day 6 (days post implant). Treatment was initiated on day 7 (days post implant), where tumors were ˜100 mm³. DGKi of formula II from the group of compounds 17-34 was formulated in 90% PEG400, 5% Ethanol, and 5% TPGS and given orally at a volume of 10 mL/kg body weight, every day for a total of 28 doses (QDx28) at 0.3 mg/kg. Anti-PD-1 (mIgG1-D265A monoclonal antibody directed against mouse PD-1), anti-CTLA4 (mIgG2b monoclonal antibody directed against mouse CTLA4) and corresponding isotype controls (InVivoPlus Mouse IgG1, clone MOPC-21, and InVivoMab Mouse IgG2b, clone MPC-11, (both from Bio X Cell (West Lebanon, N.H.) isotype controls for anti-PD-1 and anti-CTLA4, respectively) were diluted with DPBS to a dose of 10 mg/kg. Antibody therapies were administered via intraperitoneal injection (I.P.), every 4 days for a total of 3 doses (Q4Dx3). Tumor volumes were measured twice a week with a digital caliper until tumors had completely regressed (0 mm³) or reached 1000 mm³ and were euthanized.

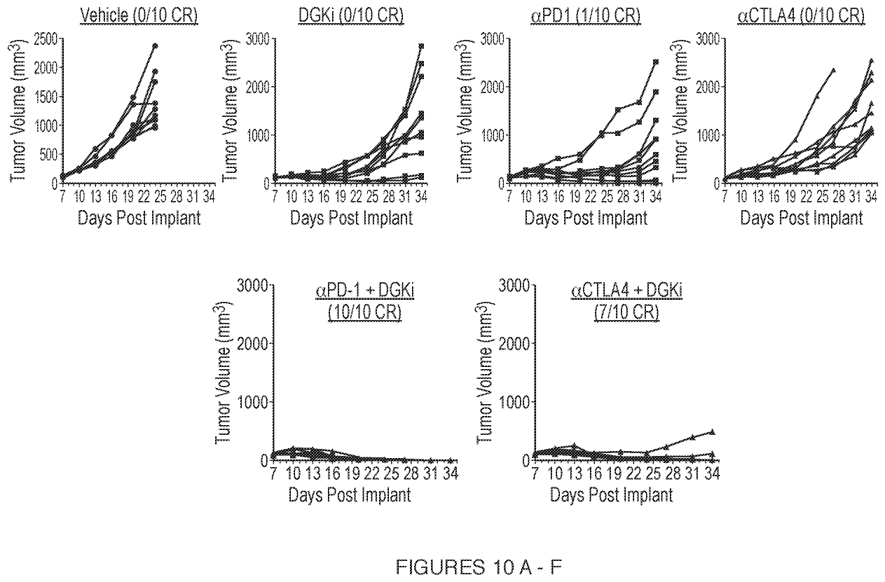

The results, which are shown in FIG. 10, indicate that single agents elicited modest efficacy (FIG. 10 B-D), but that combinations of the DGKi with anti-PD-1 or anti-CTLA4 antibody elicits robust anti-tumor activity (FIGS. 10 E and F, respectively).

### Example 11: Combination of a Compound of Formula II with an Anti-PD-1 Antibody Shows Strong Anti-Tumor Efficacy and Durable Immunological Memory in Both the MC38 and CT26 Animal Models

This Example shows that an exemplary DGKi of formula II from the group of compounds 17-34 together with anti-PD-1 has strong anti-tumor efficacy that can elicit complete tumor regression and durable immunological memory in both the MC38 and CT26 animal models.

The study was conducted as follows. The MC38 animal model study was conducted as described in Example 10. The CT26 study was conducted as illustrated in Example 3. DGKi and anti-PD-1 (same as in Example 10) were prepared and administered as in Example 10. Cured animals from the original treatment paradigm were retained after change in tumor volume was stagnant for 10×TVDT (10×4.2 days=42 days). These animals were implanted with I0×the initial cell concentration subcutaneously into the right flank. These animals were measured twice weekly for another period of 42+ days to evaluate T cell memory response.

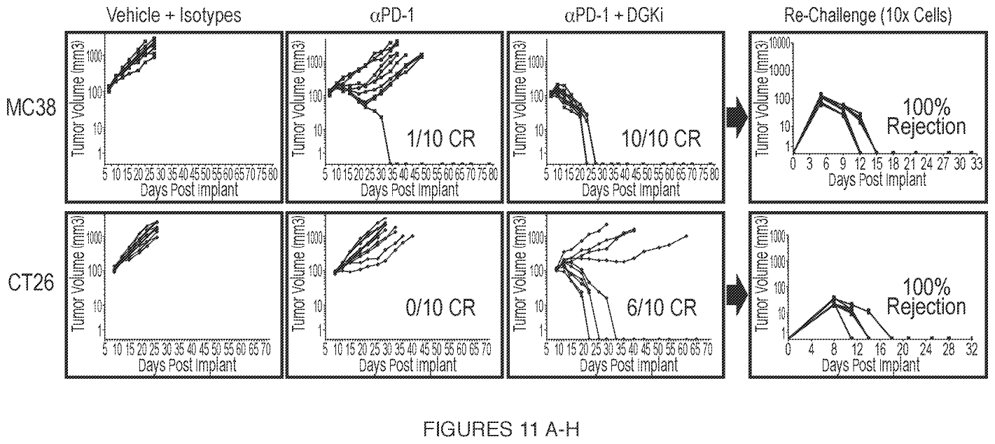

The results, which are shown in FIG. 11 A-H, indicate that the combination of a DGKi of formula II with an anti-PD-1 antibody results in a strong anti-tumor effect in the animal models tested. In addition, the re-challenge with tumor cells in the MC38 and CT26 models led to 100% rejection of the transplanted cells (FIGS. 11 D and H).

### Example 12: Combination of a Compound of Formula II with Anti-PD-1 and Anti-CTLA4 Provides Stronger Efficacy Relative to Combinations with Anti-PD-1 or Anti-CTLA4 in the B16F10 Animal Model

This Example shows that a triple combination of an exemplary DGKi of formula II from the group of compounds 17-34 with an anti-PD-1 antibody and an anti-CTLA4 antibody generates an anti-tumor effect that is stronger than that of the double combinations in the B16F10 (melanoma/MHCI^{lo}) animal model.

The animal model study was conducted as follows. The mouse melanoma tumor cell line B16F10 was maintained in 10% fetal bovine serum (FBS, Invitrogen) and Dulbecco's Modified Eagle Medium (DMEM) (Gibco) in T75 flasks. Cells were grown to subconfluency and passaged two times per week simply by rinsing flask with DPBS (Dulbecco's Phosphate-Buffered Saline, Gibco), then rinsing flask with trypsin (0.25% trypsin, Gibco), allowing cells to sit for a few minutes and tapping the flask. B16F10 cell passage ratios ranged from 1:18 to −1:20 depending on timing and confluency. For in vivo implantation, cells were trypsinized as above and then collected in ice-cold HBSS (Hank's Balanced Salt Solution, Gibco) in 50 mL conical tubes on ice. Tubes were spun at 1300 rpm for 10 minutes, the supernatant carefully removed, and the pellets washed with HBSS and spun again. Pellets were resuspended in approximate implant volumes of HBSS. The cell concentration was measured using a Moxi-Z (Orflo) and adjusted to the final concentration with HBSS. Cell viability was measured using trypan blue exclusion on a Countess II (Life Technologies). Female C57Bl/6 mice, obtained from Charles River Laboratories (Raleigh, N.C.), were received in house at age 6-8 weeks and acclimated for 3-7 days. At the time of tumor implantation (day 0), mice were given a subcutaneous injection of 0.1 mL of B16.F10 cells at a concentration of 1×10⁷ cells/ml using a 1 mL tuberculin syringe with a 25 gauge needle, implanted into the right flank. Treatment was initiated when tumors grew to a pre-determined size, ˜50 mm³, at which time animals were randomized into various treatment and control groups with like mean and median tumor values, with n=10 per group on day 8 (days post implant). DGKi of formula II from the group of compounds 17-34 was formulated in 90% PEG400, 5% Ethanol, and 5% TPGS and given orally at a volume of 10 mL/kg body weight, every day for a total of 28 doses (QDx28) at 0.3 mg/kg. Anti-PD-1, anti-CTLA4 and corresponding isotype controls (same as in Example 10) were diluted with DPBS to a dose of 10 mg/kg. Antibody therapies were administered via intraperitoneal injection (I.P.), every 4 days for a total of 3 doses (Q4Dx3). Tumor volumes were measured twice a week with a digital caliper until tumors had completely regressed (0 mm³) or reached 1000 mm³ and were euthanized.

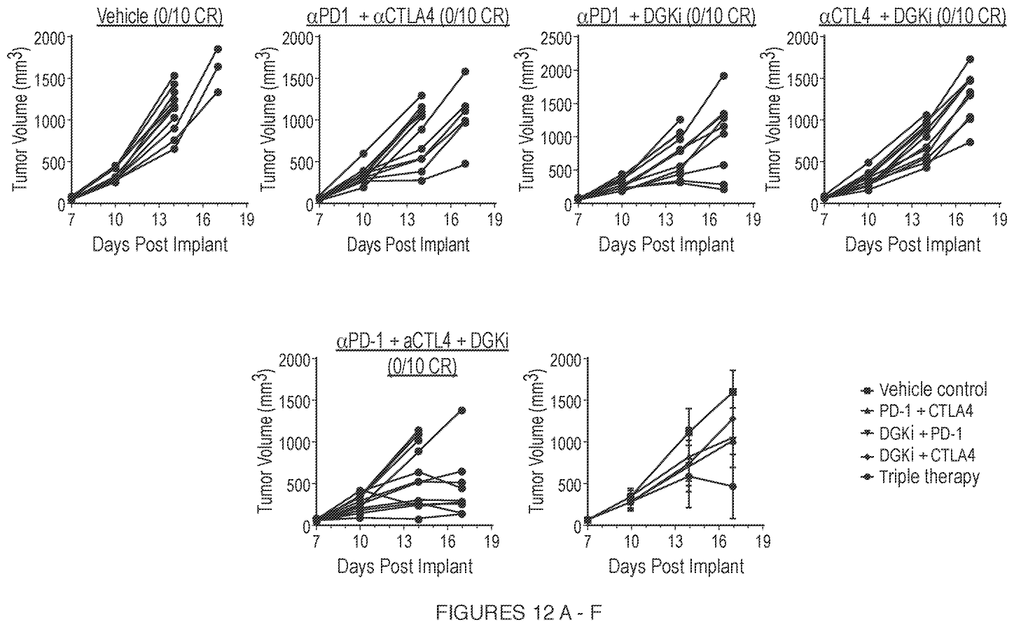

The results, which are shown in FIG. 12 A-F, indicate that triple therapy improved the response relative to the double therapies in the B16F10 animal model.

### Example 13: Synthesis of DGK Inhibitors

### DGKi Compound 1

### 4-((2R,5S)-4-(bis(4-fluorophenyl)methyl)-2,5-dimethylpiperazin-1-yl)-6-bromo-1-methyl-2-oxo-1,2-dihydro-1,5-naphthyridine-3-carbonitrile

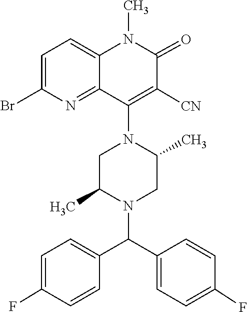 [SMILES: C[C@@H]1CN(C(c2ccc(F)cc2)c2ccc(F)cc2)[C@@H](C)CN1c1c(C#N)c(=O)n(C)c2ccc(Br)nc12]

### DGKi Compound 2

### Methyl 1-(bis(4-fluorophenyl)methyl)-4-(6-cyano-1-methyl-2-oxo-1,2-dihydro-1,5-naphthyridin-4-yl)piperazine-2-carboxylate

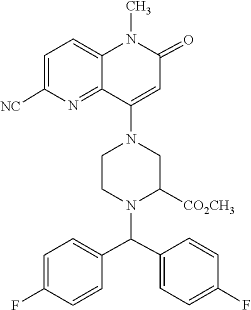 [SMILES: COC(=O)C1CN(c2cc(=O)n(C)c3ccc(C#N)nc23)CCN1C(c1ccc(F)cc1)c1ccc(F)cc1]

### DGKi Compound 3

### (R)-4-(4-(bis(4-fluorophenyl)methyl)-3-methylpiperazin-1-yl)-6-bromo-1-methyl-2-oxo-1,2-dihydro-1,5-naphthyridine-3-carbonitrile

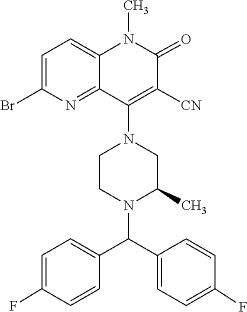 [SMILES: C[C@@H]1CN(c2c(C#N)c(=O)n(C)c3ccc(Br)nc23)CCN1C(c1ccc(F)cc1)c1ccc(F)cc1]

### DGKi Compound 4

### (R)-8-(4-(bis(4-fluorophenyl)methyl)-3-methylpiperazin-1-yl)-5-methyl-6-oxo-5,6-dihydro-1,5-naphthyridine-2,7-dicarbonitrile

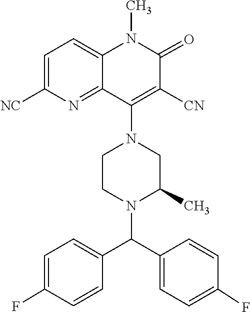 [SMILES: C[C@@H]1CN(c2c(C#N)c(=O)n(C)c3ccc(C#N)nc23)CCN1C(c1ccc(F)cc1)c1ccc(F)cc1]

### DGKi Compound 5

### 8-[(2S,5R)-4-[(4-fluorophenyl)(phenyl)methyl]-2,5-dimethylpiperazin-1-yl]-5-methyl-6-oxo-5,6-dihydro-1,5-naphthyridine-2-carbonitrile

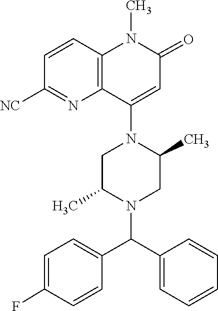 [SMILES: C[C@@H]1CN(c2cc(=O)n(C)c3ccc(C#N)nc23)[C@@H](C)CN1C(c1ccccc1)c1ccc(F)cc1]

### DGKi Compounds 6 and 7

### 8-[(2S,5R)-4-[(4-fluorophenyl)(phenyl)methyl]-2,5-dimethylpiperazin-1-yl]-5-methyl-6-oxo-5,6-dihydro-1,5-naphthyridine-2-carbonitrile

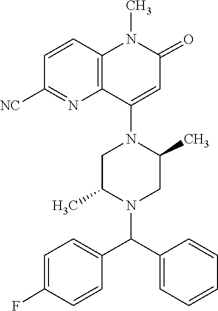 [SMILES: C[C@@H]1CN(c2cc(=O)n(C)c3ccc(C#N)nc23)[C@@H](C)CN1C(c1ccccc1)c1ccc(F)cc1]

### DGKi Compound 8

### 4-[(2S,5R)-4-[(4-chlorophenyl)(4-fluorophenyl)methyl]-2,5-dimethylpiperazin-1-yl]-6-methoxy-1-methyl-1,2-dihydro-1,5-naphthyridin-2-one

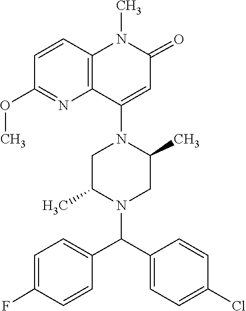 [SMILES: COc1ccc2c(n1)c(N1C[C@@H](C)N(C(c3ccc(F)cc3)c3ccc(Cl)cc3)C[C@@H]1C)cc(=O)n2C]

### DGKi Compound 9

### 8-[(2S,5R)-4-{[2-(difluoromethyl)-4-fluorophenyl]methyl}-2,5-dimethylpiperazin-1-yl]-5-methyl-6-oxo-5,6-dihydro-1,5-naphthyridine-2-carbonitrile

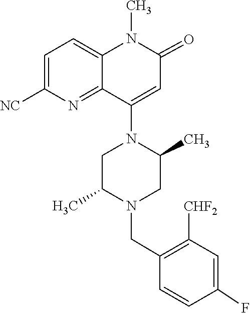 [SMILES: C[C@@H]1CN(c2cc(=O)n(C)c3ccc(C#N)nc23)[C@@H](C)CN1Cc1ccc(F)cc1C(F)F]

### DGKi Compound 10

### 8-[(2S,5R)-4-[(4-fluorophenyl)(4-methylphenyl)methyl]-2,5-dimethylpiperazin-1-yl]-5-methyl-6-oxo-5,6-dihydro-1,5-naphthyridine-2-carbonitrile

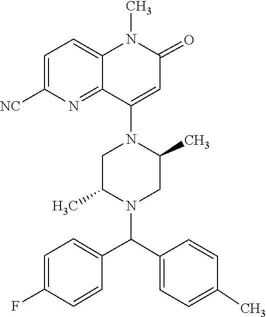 [SMILES: Cc1ccc(C(c2ccc(F)cc2)N2C[C@H](C)N(c3cc(=O)n(C)c4ccc(C#N)nc34)C[C@H]2C)cc1]

### DGKi Compound 11

### 8-[(2S,5R)-4-[1-(2,6-difluorophenyl)ethyl]-2,5-dimethylpiperazin-1-yl]-5-methyl-6-oxo-5,6-dihydro-1,5-naphthyridine-2-carbonitrile

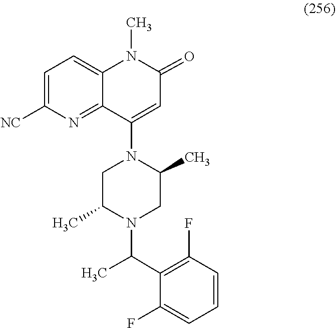 [SMILES: CC(c1c(F)cccc1F)N1C[C@H](C)N(c2cc(=O)n(C)c3ccc(C#N)nc23)C[C@H]1C]

### DGKi Compounds 12-14

### 8-((2S,5R)-4-(1-(2,4-difluorophenyl)propyl)-2,5-dimethylpiperazin-1-yl)-5-methyl-6-oxo-5,6-dihydro-1,5-naphthyridine-2-carbonitrile

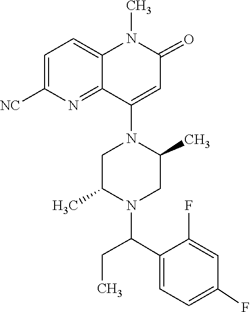 [SMILES: CCC(c1ccc(F)cc1F)N1C[C@H](C)N(c2cc(=O)n(C)c3ccc(C#N)nc23)C[C@H]1C]

### Intermediate 1

### Ethyl 6-cyano-3-(N-methylacetamido)picolinate

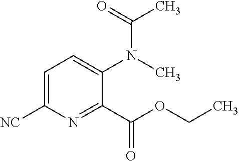 [SMILES: CCOC(=O)c1nc(C#N)ccc1N(C)C(C)=O]

To a stirred pale yellow solution of ethyl 3-(N-methylacetamido)-1-(l1-oxidanyl)-114-pyridine-2-carboxylate (50 g, 210 mmol) in DCM (500 mL) at room temperature was added trimethylsilyl cyanide (39.4 mL, 294 mmol). The reaction mixture was stirred for 10 min and cooled the mixture to −10° C. Next, benzoyl chloride (34.1 mL, 294 mmol) was added through a 50 mL addition funnel over 15 min followed by TEA (41.0 mL, 294 mmol) through a 50 mL addition funnel slowly over 20 min. An exothermic reaction was observed during TEA addition. The reaction mixture turned to a turbid mixture (TEA salt) which was stirred for 2.5 h at the same temperature. The reaction was quenched with 10% NaHCO₃ solution (500 mL) and extracted with DCM (3×300 mL). The combined organic solution was washed with brine (2×250 mL) then dried over Na₂SO₄ and concentrated to yield a light yellow crude material. The crude material was purified through normal phase RediSep silica column on ISCO@ using EA/petroleum ether as eluent. The product was isolated by 65-70% EA/petroleum ether, fractions were concentrated to afford ethyl 6-cyano-3-(N-methylacetamido)picolinate (43 g, 83% yield) as a light brown liquid; LCMS: m/z=248.0 (M+H); rt 1.255 min; LC-MS Method: Column-KINETEX-XB-C18 (75×3 mm-2.6 μm); Mobile phase A: 10 mM ammonium formate in water:acetonitrile (98:2); Mobile phase B: 10 mM ammonium formate in water:acetonitrile (2:98); Gradient: 20-100% B over 4 minutes, flow rate 1.0 mL/min, then a 0.6 minute hold at 100% B flow rate 1.5 mL/min; then Gradient: 100-20% B over 0.1 minutes, flow rate 1.5 m/min.

### Intermediate 2

### 8-Hydroxy-5-methyl-6-oxo-5,6-dihydro-1,5-naphthyridine-2-carbonitrile

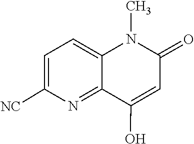

To a stirred solution of ethyl 6-cyano-3-(N-methylacetamido)picolinate (0.9 g, 3.64 mmol) in tetrahydrofuran (10 mL) was added KHMDS (4.80 mL, 4.37 mmol) at −78° C. over 10 min. The reaction mixture was stirred for 15 min. The reaction mixture was slowly warmed to room temperature over 30 min and then stirred for another 90 min. The reaction mixture was cooled to 0° C. The reaction was quenched with saturated sodium bicarbonate solution (70 mL). The mixture was diluted with ethyl acetate (2×100 mL). The aqueous layer was collected and acidified with 1.5 N HCL to adjust the pH to ˜3.0. The mixture was stirred for 15 min to form a solid mass, which was filtered through a Buchner funnel to yield 8-hydroxy-5-methyl-6-oxo-5,6-dihydro-1,5-naphthyridine-2-carbonitrile 550 mg, 75% yield) as a brown solid. LCMS: m/z=202.0 (M+H); rt 0.361 min; LC-MS Method: Column-KINETEX-XB-C18 (75×3 mm-2.6 μm); Mobile phase A: 10 mM ammonium formate in water:acetonitrile (98:2); Mobile phase B: 10 mM ammonium formate in water:acetonitrile (2:98); Gradient: 20-100% B over 4 minutes, flow rate 1.0 mL/min, then a 0.6 minute hold at 100% B flow rate 1.5 mL/min; then Gradient: 100-20% B over 0.1 minutes, flow rate 1.5 mL/min.

### Intermediate 3

### 8-Chloro-5-methyl-6-oxo-5,6-dihydro-1,5-naphthyridine-2-carbonitrile

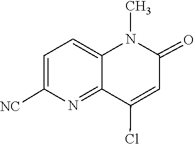

To a stirred solution of 8-hydroxy-5-methyl-6-oxo-5,6-dihydro-1,5-naphthyridine-2-carbonitrile (0.55 g, 2.73 mmol) in acetonitrile (10 mL) was added POCl₃ (1.53 mL, 16.4 mmol). The reaction mixture was heated up to 85° C. over 5 min and was stirred for 16 h. The reaction mixture was concentrated under reduced pressure to yield crude material. The reaction mixture was cooled to 0° C. The reaction was quenched with saturated sodium bicarbonate solution (50 mL). The reaction was diluted with DCM (3×100 mL). The combined organic layer was dried over anhydrous sodium sulfate, filtered, and evaporated under reduced pressure to yield 8-chloro-5-methyl-6-oxo-5,6-dihydro-1,5-naphthyridine-2-carbonitrile (0.25 g, 29.1% yield) as a brown solid. LCMS: m/z=220.2 (M+H); rt 1.528 min; LC-MS Method: Column-KINETEX-XB-C18 (75×3 mm-2.6 μm); Mobile phase A: 10 mM ammonium formate in water:acetonitrile (98:2); Mobile phase B: 10 mM ammonium formate in water:acetonitrile (2:98); Gradient: 20-100% B over 4 minutes, flow rate 1.0 mL/min, then a 0.6 minute hold at 100% B flow rate 1.5 mL/min; then Gradient: 100-20% B over 0.1 minutes, flow rate 1.5 mL/min.

### Intermediate 4

### Stereochemistry: A

### (Cyanomethyl)trimethylphosphonium iodide

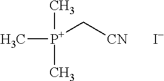

(Cyanomethyl)trimethylphosphonium iodide was prepared according to the general method described in Zaragoza, F., et al., J. Org. Chem. 2001, 66, 2518-2521. In a 1 L round bottom flask, trimethylphosphine in toluene (100 mL, 100 mmol) was diluted with THF (50.0 mL) and toluene (50.0 mL), and cooled on an ice bath. The reaction mixture was stirred vigorously while iodoacetonitrile (7 mL, 16.7 g, 68.3 mmol) was added dropwise to produce a tan precipitate. The cooling bath was removed and the reaction mixture was stirred overnight at room temperature. The flask was placed in a sonicator to break up any clumped solids. The reaction mixture was stirred an additional 4 hours. The solids were collected by filtration and dried under vacuum to give (cyanomethyl)trimethylphosphonium iodide (16.6 g, 68.3 mmol, 68.3% yield). ¹H NMR (400 MHz, DMSO-d₆) δ 4.03 (d, J=16.4 Hz, 2H), 2.05 (d, J=15.4 Hz, 9H).

### Intermediate 5

### Stereochemistry: Homochiral

### 8-((2S,5R)-2,5-Dimethylpiperazin-1-yl)-5-methyl-6-oxo-5,6-dihydro-1,5-naphthyridine-2-carbonitrile, TFA

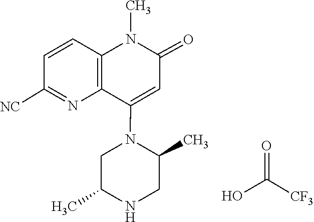 [SMILES: C[C@@H]1CN(c2cc(=O)n(C)c3ccc(C#N)nc23)[C@@H](C)CN1 ; O=C(O)C(F)(F)F]

To a solution of 6-cyano-1-methyl-2-oxo-1,2-dihydro-1,5-naphthyridin-4-yl trifluoromethanesulfonate (65 g, 195 mmol) and tert-butyl (2R,5S)-2,5-dimethylpiperazine-1-carboxylate (43.9 g, 205 mmol) in acetonitrile (1.3 L) was added DIPEA (0.102 L, 585 mmol). The solution was stirred at 80° C. for 6 hours. The solvent was removed and the crude residue chromatographed on silica gel (product R_{f} 0.4 in 100% ethyl acetate). The product, tert-butyl (2R,5S)-4-(6-cyano-1-methyl-2-oxo-1,2-dihydro-1,5-naphthyridin-4-yl)-2,5-dimethylpiperazine-1-carboxylate (75 g, 189 mmol, 97% yield) was obtained. LCMS: m/z=398.2 (M+H); rt 2.7 min. Method: Column-Kinetex XB-C18 (75×3 mm-2.6 μm), flow rate 1 mL/min; gradient time 4 min; 20% Solvent B to 100% Solvent B; monitoring at 254 nm (Solvent A: 98% water: 2% acetonitrile; 10 mM ammonium formate; Solvent B: 2% water: 98% acetonitrile; 10 mM ammonium formate.

To a solution of tert-butyl (2R,5S)-4-(6-cyano-1-methyl-2-oxo-1,2-dihydro-1,5-naphthyridin-4-yl)-2,5-dimethylpiperazine-1-carboxylate (30 g, 75 mmol) in ethyl acetate (1000 mL) at 0° C. was added HCl (4 M in dioxane) (189 mL, 755 mmol) and the temperature was allowed to reach room temperature while stirring for 6 h. LC/MS analysis showed ˜90% product mass at 0.60 RT along with ˜4% of an amide byproduct mass (consistent with nitrile hydrolysis) at 0.44 RT. The reaction mixture was diluted with methyl t-butyl ether (MTBE, 2000 mL), stirred for 15 mins, and the HCl salt of product was filtered, washed with MTBE (100 ml). The HCl salt was dissolved in water (300 ml) and the pH adjusted to ˜8 using 10% aqueous sodium bicarbonate. The organic portion was extracted with DCM (5×250 ml). The combined organic layers were washed with water (2×300 mL), dried over sodium sulphate and concentrated to afford 8-((2S,5R)-2,5-dimethylpiperazin-1-yl)-5-methyl-6-oxo-5,6-dihydro-1,5-naphthyridine-2-carbonitrile (20 g, 65.2 mmol, 86% yield). LCMS: m/z=298.2 (M+H); rt 0.5 min. Method: Column-Kinetex XB-C18 (75×3 mm-2.6 μm), flow rate 1 mL/min; gradient time 4 min; 20% Solvent B to 100% Solvent B; monitoring at 254 nm (Solvent A: 98% water: 2% acetonitrile; 10 mM ammonium formate; Solvent B: 2% water: 98% acetonitrile; 10 mM ammonium formate. ¹H NMR (400 MHz, CDCl₃) δ 7.79 (d, J=8.8 Hz, 1H), 7.70 (d, J=12, 3.2 Hz, 1H), 6.29 (s, 1H), 3.80 (dd, J=8.8 Hz, 1H) 3.70 (m, 1H), 3.65 (s, 3H), 3.29 (m, 2H), 2.80 (m, 2H), 1.19 (d, J=6 Hz, 3H), 1.15 (d, J=6 Hz, 3H). ¹³C NMR (75 MHz, Chloroform-d) δ 161.9, 155.0, 138.5, 137.0, 128.2, 125.0, 122.2, 117.2, 111.3, 56.5, 51.9, 50.0, 49.5, 29.0, 18.8, 15.4.

### Intermediate 6

### 8-Chloro-5-methyl-7-nitro-6-oxo-5,6-dihydro-1,5-naphthyridine-2-carbonitrile

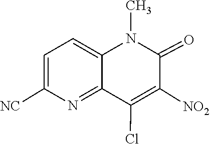 [SMILES: Cn1c(=O)c([N+](=O)[O-])c(Cl)c2nc(C#N)ccc21]

In a 2 dram vial containing 8-hydroxy-5-methyl-7-nitro-6-oxo-5,6-dihydro-1,5-naphthyridine-2-carbonitrile (192 mg, 0.780 mmol) a magnetic stir bar and acetonitrile (3.1 mL) were added. Next, DIEA (0.272 mL, 1.560 mmol) was added to the suspension. The reaction mixture was stirred for 1-2 minutes until the reaction mixture became a homogeneous yellow solution. To the reaction mixture was added phosphorous oxychloride (0.131 mL, 1.404 mmol). The vial was capped under nitrogen with vent to an oil bubbler. The reaction mixture was stirred at room temperature for 1.5 hours then benzyltriethylammonium chloride (200 mg, 0.878 mmol) was added to the reaction mixture. The vial was capped under a nitrogen atmosphere and immersed in an oil bath (65° C.) and heated for 1 hour. The reaction mixture was cooled and the reaction volatiles were remove in vacuo using a rotary evaporator. The reaction residue was dissolved in ethyl acetate, poured into a beaker containing ice (˜10 mL), and then transferred to a separatory funnel. The aqueous phase was extracted with ethyl acetate. The organic extracts combined and washed sequentially with 1.5 M K₂HPO₄, saturated aqueous sodium bicarbonate, and brine. The organic extract was dried over magnesium sulfate, filtered, and solvent removed in vacuo to give a 204 mg of a brownish crystalline solid. LCMS: Column: Waters Acquity UPLC BEH C18, 2.1×50 mm, 1.7 μm particles; Mobile Phase A: 100% water with 0.05% trifluoroacetic acid; Mobile Phase B: 100% acetonitrile with 0.05% trifluoroacetic acid; Temperature: 40° C.; Gradient: 2-98% B over 1.5 minutes, then a 0.5 minute hold at 98% B; Flow: 0.8 mL/min; Detection: UV at 220 nm. Retention Time=1.01 min.; Obs. Adducts: [M+H]; Obs. Masses: 265.0 (weak ionization). ¹H NMR (CHLOROFORM-d) δ 8.03 (d, J=8.8 Hz, 1H), 7.89-7.97 (m, 1H), 3.82 (s, 3H).

### Intermediate 7

### 8-((2S,5R)-2,5-Dimethylpiperazin-1-yl)-5-methyl-6-oxo-5,6-dihydro-1,5-naphthyridine-2-carbonitrile, TFA

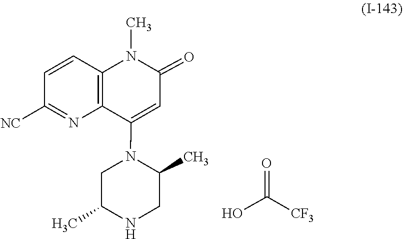 [SMILES: C[C@@H]1CN(c2cc(=O)n(C)c3ccc(C#N)nc23)[C@@H](C)CN1 ; O=C(O)C(F)(F)F]

To a solution of 6-cyano-1-methyl-2-oxo-1,2-dihydro-1,5-naphthyridin-4-yl trifluoromethanesulfonate (65 g, 195 mmol) and tert-butyl (2R,5S)-2,5-dimethylpiperazine-1-carboxylate (43.9 g, 205 mmol) in acetonitrile (1.3 L) was added DIPEA (0.102 L, 585 mmol). The solution was stirred at 80° C. for 6 hours. The solvent was removed and the crude residue chromatographed on silica gel (product R_{f} 0.4 in 100% ethyl acetate). The product, tert-butyl (2R,5S)-4-(6-cyano-1-methyl-2-oxo-1,2-dihydro-1,5-naphthyridin-4-yl)-2,5-dimethylpiperazine-1-carboxylate (75 g, 189 mmol, 97% yield) was obtained. LCMS: m/z=398.2 (M+H); rt 2.7 min. Method: Column-Kinetex XB-C18 (75×3 mm-2.6 μm), flow rate 1 mL/min; gradient time 4 min; 20% Solvent B to 100% Solvent B; monitoring at 254 nm (Solvent A: 98% water: 2% acetonitrile; 10 mM ammonium formate; Solvent B: 2% water: 98% acetonitrile; 10 mM ammonium formate.

To a solution of tert-butyl (2R,5S)-4-(6-cyano-1-methyl-2-oxo-1,2-dihydro-1,5-naphthyridin-4-yl)-2,5-dimethylpiperazine-1-carboxylate (30 g, 75 mmol) in ethyl acetate (1000 mL) at 0° C. was added HCl (4 M in dioxane) (189 mL, 755 mmol) and the temperature was allowed to reach room temperature while stirring for 6 h. LC/MS analysis showed ˜90% product mass at 0.60 RT along with ˜4% of an amide byproduct mass (consistent with nitrile hydrolysis) at 0.44 RT. The reaction mixture was diluted with methyl t-butyl ether (MTBE, 2000 mL), stirred for 15 mins, and the HCl salt of product was filtered, washed with MTBE (100 ml). The HCl salt was dissolved in water (300 ml) and the pH adjusted to ˜8 using 10% aqueous sodium bicarbonate. The organic portion was extracted with DCM (5×250 mL). The combined organic layers were washed with water (2×300 mL), dried over sodium sulphate and concentrated to afford 8-((2S,5R)-2,5-dimethylpiperazin-1-yl)-5-methyl-6-oxo-5,6-dihydro-1,5-naphthyridine-2-carbonitrile (20 g, 65.2 mmol, 86% yield). LCMS: m/z=298.2 (M+H); rt 0.5 min. Method: Column-Kinetex XB-C18 (75×3 mm-2.6 μm), flow rate 1 mL/min; gradient time 4 min; 20% Solvent B to 100% Solvent B; monitoring at 254 nm (Solvent A: 98% water: 2% acetonitrile; 10 mM ammonium formate; Solvent B: 2% water: 98% acetonitrile; 10 mM ammonium formate. ¹H NMR (400 MHz, CDCl₃) δ 7.79 (d, J=8.8 Hz, 1H), 7.70 (d, J=12, 3.2 Hz, 1H), 6.29 (s, 1H), 3.80 (dd, J=8.8 Hz, 1H) 3.70 (m, 1H), 3.65 (s, 3H), 3.29 (m, 2H), 2.80 (m, 2H), 1.19 (d, J=6 Hz, 3H), 1.15 (d, J=6 Hz, 3H). ¹³C NMR (75 MHz, Chloroform-d) δ 161.9, 155.0, 138.5, 137.0, 128.2, 125.0, 122.2, 117.2, 111.3, 56.5, 51.9, 50.0, 49.5, 29.0, 18.8, 15.4. Stereochemistry: Homochiral.

### Method of Synthesizing DGKi Compound 1

### 4-((2R,5S)-4-(bis(4-fluorophenyl)methyl)-2,5-dimethylpiperazin-1-yl)-6-bromo-1-methyl-2-oxo-1,2-dihydro-1,5-naphthyridine-3-carbonitrile

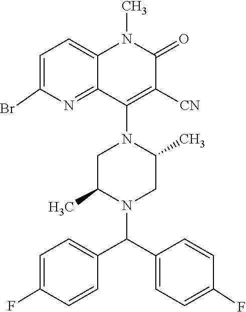 [SMILES: C[C@@H]1CN(C(c2ccc(F)cc2)c2ccc(F)cc2)[C@@H](C)CN1c1c(C#N)c(=O)n(C)c2ccc(Br)nc12]

To a stirred solution of 6-bromo-3-cyano-1-methyl-2-oxo-1,2-dihydro-1,5-naphthyridin-4-yl trifluoromethanesulfonate (80 mg, 0.194 mmol) in acetonitrile (5 mL) were added DIPEA (0.102 mL, 0.582 mmol) and HCl salt of (2S,5R)-1-(bis(4-fluorophenyl)methyl)-2,5-dimethylpiperazine (75 mg, 0.214 mmol). The reaction mixture was stirred at 85° C. overnight. The solvent was removed under reduced pressure and the residue was dissolved in ethyl acetate (15 mL). The organic layer was washed with brine, dried over Na₂SO₄ and concentrated under reduced pressure. The crude residue purified by silica gel column chromatography using 24 g flash column, eluting with 50-80% EtOAc in petroleum ether. The fractions were concentrated under reduced pressure to yield 4-((2R,5S)-4-(bis(4-fluorophenyl)methyl)-2,5-dimethylpiperazin-1-yl)-6-bromo-1-methyl-2-oxo-1,2-dihydro-1,5-naphthyridine-3-carbonitrile (95 mg, 85% yield); LCMS: m/z=578.2 (M+H); rt 3.916 min.

### Method of Synthesizing DGKi Compound 2

### Methyl 1-(bis(4-fluorophenyl)methyl)-4-(6-cyano-1-methyl-2-oxo-1,2-dihydro-1,5-naphthyridin-4-yl)piperazine-2-carboxylate

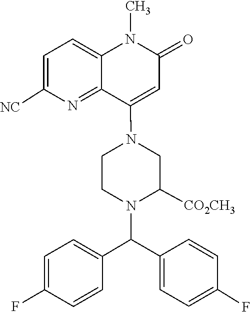 [SMILES: COC(=O)C1CN(c2cc(=O)n(C)c3ccc(C#N)nc23)CCN1C(c1ccc(F)cc1)c1ccc(F)cc1]

To a stirred solution of 8-chloro-5-methyl-6-oxo-5,6-dihydro-1,5-naphthyridine-2-carbonitrile (22.90 mg, 0.104 mmol) in DMA (1 mL) and t-butanol (4 mL) was added the TFA salt of methyl 1-(bis(4-fluorophenyl)methyl)piperazine-2-carboxylate (40 mg, 0.087 mmol) and cesium carbonate (85 mg, 0.261 mmol) under a nitrogen atmosphere, followed by the addition of chloro(2-dicyclohexylphosphino-2′,6′-diisopropoxy-1,1′-biphenyl)[2-(2′-amino-1,1′-biphenyl)]palladium(II) (3.37 mg, 4.34 μmol). The reaction vessel was immersed in an oil bath at 70° C. The bath temperature was raised to 90° C. over 2 min and the reaction mixture was stirred for 16 h. The reaction mixture was filtered through a celite bed and was concentrated under high vacuum to yield a brown gum. The crude material was purified via preparative HPLC with the following conditions: Column: Sunfire C18, 19×150 mm, 5 μm particles; Mobile Phase A: 10 mM ammonium acetate pH 4.5 with acetic acid; Mobile Phase B: acetonitrile; Gradient: 30-100% B over 15 minutes, then a 5 minute hold at 100% B; Flow: 17 mL/min. Fractions containing the product were combined and dried via centrifugal evaporation to yield methyl 1-(bis(4-fluorophenyl)methyl)-4-(6-cyano-1-methyl-2-oxo-1,2-dihydro-1,5-naphthyridin-4-yl)piperazine-2-carboxylate (3.5 mg, 6.23 μmol, 7.17% yield). LCMS: m/z=530.2 (M+H); rt 2.20 min. LC-MS Method: Column-X Bridge BEH XP C18 (50×2.1 mm 2.5 μm; flow rate 1.1 mL/min; gradient time 3 min; Temperature: 50° C., 0% Solvent B to 100% Solvent B; monitoring at 220 nm (Solvent A: 95% water: 5% acetonitrile; 10 mM ammonium acetate; Solvent B: 5% water: 95% acetonitrile; 10 mM ammonium acetate). ¹H NMR (400 MHz, DMSO-d₆) δ ppm 8.16 (d, J=8.8 Hz, 1H), 8.08 (d, J=9.0 Hz, 1H), 7.57 (dd, J=8.8, 5.6 Hz, 2H), 7.42-7.28 (m, 2H), 7.22-7.08 (m, 4H), 6.14 (s, 1H), 5.17 (s, 1H), 4.78 (d, J=12.2 Hz, 1H), 3.64 (d, J=12.0 Hz, 1H), 3.59 (s, 3H), 3.54 (s, 3H), 3.45-3.35 (m, 2H), 3.15 (dd, J=12.5, 3.9 Hz, 1H), 3.04 (td, J=11.7, 2.9 Hz, 1H), 2.71-2.63 (m, 1H).

### Method of Synthesizing DGKi Compound 3

### (R)-4-(4-(bis(4-fluorophenyl)methyl)-3-methylpiperazin-1-yl)-6-bromo-1-methyl-2-oxo-1,2-dihydro-1,5-naphthyridine-3-carbonitrile

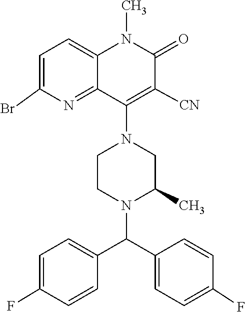 [SMILES: C[C@@H]1CN(c2c(C#N)c(=O)n(C)c3ccc(Br)nc23)CCN1C(c1ccc(F)cc1)c1ccc(F)cc1]

To a stirred solution of 6-bromo-3-cyano-1-methyl-2-oxo-1,2-dihydro-1,5-naphthyridin-4-yl trifluoromethanesulfonate (100 mg, 0.243 mmol) in acetonitrile (8 mL) were added DIPEA (0.127 mL, 0.728 mmol) and HCl salt of (R)-1-(bis(4-fluorophenyl)methyl)-2-methylpiperazine (82 mg, 0.243 mmol). The reaction mixture was heated up to 85° C. over 5 min and was stirred for 1 h. The reaction mixture was concentrated under high vacuum to yield a brown gum. The crude compound was purified by ISCO® using 12 g silica gel column; 60-67% ethyl acetate/petroleum ether to yield (R)-4-(4-(bis(4-fluorophenyl)methyl)-3-methylpiperazin-1-yl)-6-bromo-1-methyl-2-oxo-1,2-dihydro-1,5-naphthyridine-3-carbonitrile (90 mg, 42.7% yield) as a brown gum; LCMS: m/z=566.0 (M+2H); rt 2.23 min. LC-MS Method: Column-AQUITY UPLC BEH C18 (3.0×50 mm) 1.7 μm; Mobile phase A: Buffer: acetonitrile (95:5); Mobile phase B: Buffer: acetonitrile (5:95), Buffer: 10 mM ammonium acetate; Gradient: 20-100% B over 2.0 minutes, then a 0.2 minute hold at 100% B, flow rate 0.7 mL/min.

### Method of Synthesizing DGKi Compound 4

### (R)-8-(4-(bis(4-fluorophenyl)methyl)-3-methylpiperazin-1-yl)-5-methyl-6-oxo-5,6-dihydro-1,5-naphthyridine-2,7-dicarbonitrile

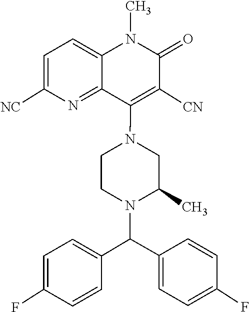 [SMILES: C[C@@H]1CN(c2c(C#N)c(=O)n(C)c3ccc(C#N)nc23)CCN1C(c1ccc(F)cc1)c1ccc(F)cc1]

To a stirred solution of (R)-4-(4-(bis(4-fluorophenyl)methyl)-3-methylpiperazin-1-yl)-6-bromo-1-methyl-2-oxo-1,2-dihydro-1,5-naphthyridine-3-carbonitrile (90 mg, 0.159 mmol) in NMP (5 mL) were added zinc (2.085 mg, 0.032 mmol) and zinc cyanide (37.4 mg, 0.319 mmol) under nitrogen. The nitrogen purging was continued for 3 min and dppf (5.30 mg, 9.57 μmol) and Pd₂(dba)₃ (14.6 mg, 0.016 mmol) were added. The reaction mixture was heated up to 80° C. over 5 min and was stirred for 4 h. The reaction mixture was filtered through celite bed and was concentrated under high vacuum to yield a brown gum. The crude material was purified via preparative HPLC. HPLC Method: Column-SUNFIRE C18 (150 mm×19 mm ID, 5 μm); Mobile phase A: 10 mM Ammonium acetate in water; Mobile phase B: acetonitrile; Gradient: 40-60% B over 3.0 minutes, flow rate 17 mL/min, then a 17 minute hold at 60-100% B flow rate 17 mL/min. Fractions containing the product were combined and concentrated under high vacuum. Then sample was diluted with (EtOH\H₂O, 1:3) and was lyophilized overnight to yield (R)-8-(4-(bis(4-fluorophenyl)methyl)-3-methylpiperazin-1-yl)-5-methyl-6-oxo-5,6-dihydro-1,5-naphthyridine-2,7-dicarbonitrile (50 mg, 61.4% yield) as pale yellow solid. LCMS: m/z=511.2 (M+H); rt 3.520 min. LC-MS Method: Column-KINETEX-XB-C18 (75×3 mm-2.6p); Mobile phase A: 10 mM ammonium formate in water:acetonitrile (98:2); Mobile phase B: 10 mM ammonium formate in water:acetonitrile (2:98); Gradient: 20-100% B over 4 minutes, flow rate 1.0 mL/min, then a 0.6 minute hold at 100% B flow rate 1.5 mL/min; then Gradient: 100-20% B over 0.1 minutes, flow rate 1.5 mL/min. ¹H NMR (400 MHz, DMSO-d₆) δ ppm 8.26 (d, J=8.8 Hz, 1H), 8.15 (d, J=9.0 Hz, 1H), 7.56 (dd, J=11.9, 8.7 Hz, 2H), 7.57 (dd, J=11.7, 8.8 Hz, 2H), 7.16 (t, J=8.9 Hz, 4H), 4.90 (s, 1H), 4.10 (d, J=13.0 Hz, 1H), 4.01 (d, J=12.5 Hz, 1H), 3.86 (dd, J=12.2, 2.9 Hz, 1H), 3.66-3.55 (m, 1H), 3.53 (s, 3H), 3.08-2.97 (m, 1H), 2.97-2.90 (m, 1H), 2.90 (s, 1H), 1.03 (d, J=6.6 Hz, 3H).

### Method of Synthesizing DGKi Compound 5

### 8-[(2S,5R)-4-[(4-fluorophenyl)(phenyl)methyl]-2,5-dimethylpiperazin-1-yl]-5-methyl-6-oxo-5,6-dihydro-1,5-naphthyridine-2-carbonitrile

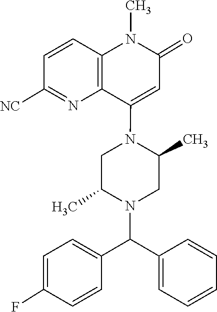 [SMILES: C[C@@H]1CN(c2cc(=O)n(C)c3ccc(C#N)nc23)[C@@H](C)CN1C(c1ccccc1)c1ccc(F)cc1]

In a 2 dram sealed reaction vessel, 8-((2S,5R)-2,5-dimethylpiperazin-1-yl)-5-methyl-6-oxo-5,6-dihydro-1,5-naphthyridine-2-carbonitrile, TFA (41.1 mg, 100 μmol), (4-fluorophenyl)(phenyl)methanol (28.3 mg, 140 μmol) and (cyanomethyl)trimethylphosphonium iodide (48.6 mg, 200 μmol) were combined in acetonitrile (200 μl). Hunig's Base (75 μL, 429 μmol) was added and the reaction mixture was heated at 110° C. for 2 hours. The reaction mixture was injected directly onto a 12 g silica gel column and eluted with 20-100% ethyl acetate in hexanes to afford Example 182 as a diasteromeric mixture. Analytical LCMS conditions: Injection Vol=3 VL, Start % B 0, Final % B 100, Gradient Time 2 Minutes, Flow Rate 1 mL/min, Wavelength 220 nm, Solvent Pair acetonitrile/Water/TFA, Solvent A 10% acetonitrile, 90% water/0.05% TFA, Solvent B 10% Water, 90% acetonitrile/0.05% TFA, Column Acquity BEH C18 21. ×50 mm 1.7 Vm, Oven Temp=40° C. LCMS results; retention time 1.4 minutes, observed mass 482.5 (M⁺).

The crude material was further purified via preparative LC/MS with the following conditions: Column: XBridge C18, 200 mm×19 mm, 5 μm particles; Mobile Phase A: 5:95 acetonitrile:water with 10 mM ammonium acetate; Mobile Phase B: 95:5 acetonitrile:water with 10 mM ammonium acetate; Gradient: a 0 minute hold at 47% B, 47-87% B over 20 minutes, then a 4 minute hold at 100% B; Flow Rate: 20 mL/min; Column Temperature: 25° C. Fraction collection was triggered by MS signals. Fractions containing the product were combined and dried via centrifugal evaporation to afford 14.4 mg of the title compound (30% yield). Calculated molecular weight 481.575. LC\MS conditions QC-ACN-TFA-XB: Observed MS Ion 482.2, retention time 1.6 minutes. ¹H NMR (500 MHz, DMSO-d₆) δ 8.18-8.10 (m, 1H), 8.06 (d, J=8.8 Hz, 1H), 7.68-7.48 (m, 4H), 7.39-7.26 (m, 2H), 7.25-7.08 (m, 3H), 6.00 (s, 1H), 4.67 (br s, 1H), 4.59 (br d, J=6.7 Hz, 1H), 3.76-3.62 (m, 1H), 3.55 (br d, J=12.8 Hz, 1H), 3.15-3.04 (m, 1H), 2.90-2.81 (m, 1H), 2.36 (br dd, J=17.4, 11.9 Hz, 1H), 1.35-1.28 (m, 3H), 1.24 (s, 1H), 1.07 (br t, J=5.6 Hz, 3H).

### Method of Synthesizing DGKi Compounds 6 and 7

### 8-[(2S,5R)-4-[(4-fluorophenyl)(phenyl)methyl]-2,5-dimethylpiperazin-1-yl]-5-methyl-6-oxo-5,6-dihydro-1,5-naphthyridine-2-carbonitrile

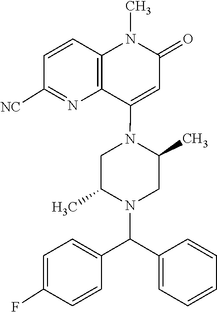 [SMILES: C[C@@H]1CN(c2cc(=O)n(C)c3ccc(C#N)nc23)[C@@H](C)CN1C(c1ccccc1)c1ccc(F)cc1]

Example 5 was separated into individual diastereomers using chiral solid phase chromatography: Column: Chiralpak OJ-H, 21×250 mm; 5 micron, Mobile Phase: 90% CO₂/10% methanol, Flow Conditions: 45 m/min, Detector Wavelength: 225 nm, Injection Details: 500 μL, 15 mg dissolved in 1 mL methanol/acetonitrile.

The first eluting diastereomer, Example 6 (66.4 mg), was isolated in 20.2% yield. Analytical LC/MS was used to determine the final purity. Injection 1 conditions: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm). Injection 1 results: Purity: 100.0%; Observed Mass: 482.1; Retention Time: 2.49 min. Injection 2 conditions: Column: Waters XBridge C18, 2.1 mm×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0% B to 100% B over 3 min, then a 0.50 min hold at 100% B; Flow: 1 mL/min; Detection: MS and UV (220 nm). Injection 2 results: Purity: 100.0%; Observed Mass: 482.11; Retention Time: 1.75 min.

The second eluting diastereomer, Example 7 (71.7 mg), was isolated in 21.9% yield. Analytical LC/MS was used to determine the final purity. Injection 1 conditions: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm). Injection 1 results: Purity: 100.0%; Observed Mass: 482.11; Retention Time: 2.51 min. Injection 2 conditions: Column: Waters XBridge C18, 2.1 mm×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0% B to 100% B over 3 min, then a 0.50 min hold at 100% B; Flow: 1 mL/min; Detection: MS and UV (220 nm). Injection 2 results: Purity: 100.0%; Observed Mass: 482.1; Retention Time: 1.76 min.

### Method of Synthesizing DGKi Compound 8

### 4-[(2S,5R)-4-[(4-chlorophenyl)(4-fluorophenyl)methyl]-2,5-dimethylpiperazin-1-yl]-6-methoxy-1-methyl-1,2-dihydro-1,5-naphthyridin-2-one

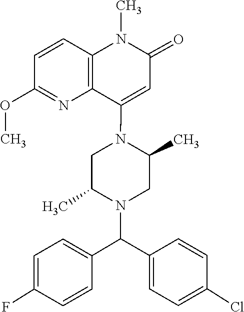 [SMILES: COc1ccc2c(n1)c(N1C[C@@H](C)N(C(c3ccc(F)cc3)c3ccc(Cl)cc3)C[C@@H]1C)cc(=O)n2C]

4-((2S,5R)-2,5-dimethylpiperazin-1-yl)-6-methoxy-1-methyl-1,5-naphthyridin-2(1H)-one (50 mg, 0.165 mmol) and 1-(bromo(4-chlorophenyl)methyl)-4-fluorobenzene (49.5 mg, 0.165 mmol) were combined with diisopropyl ethyl amine (0.173 mL, 0.992 mmol) in acetonitrile (3 mL) and the reaction mixture was heated at 55° C. overnight. LC/MS indicated the reaction was completed. The crude material was purified via preparative LC/MS with the following conditions: Column: XBridge C18, 200 mm×19 mm, 5 μm particles; Mobile Phase A: 5:95 acetonitrile:water with 10 mM ammonium acetate; Mobile Phase B: 95:5 acetonitrile:water with 10 mM ammonium acetate; Gradient: a 0 minute hold at 42% B, 42-82% B over 25 minutes, then a 5 minute hold at 100% B; Flow Rate: 20 mL/min; Column Temperature: 25° C. Fraction collection was triggered by MS signals. Fractions containing the product were combined and dried via centrifugal evaporation. Calculated molecular weight 521.03. LCMS conditions QC-ACN-AA-XB: Observed MS Ion 521.1, retention time 2.77 minutes.

### Method of Synthesizing DGKi Compound 9

### 8-[(2S,5R)-4-{[2-(difluoromethyl)-4-fluorophenyl]methyl}-2,5-dimethylpiperazin-1-yl]-5-methyl-6-oxo-5,6-dihydro-1,5-naphthyridine-2-carbonitrile

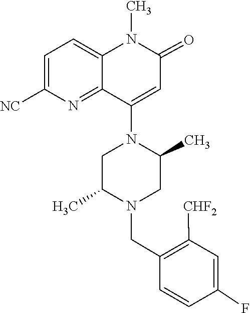 [SMILES: C[C@@H]1CN(c2cc(=O)n(C)c3ccc(C#N)nc23)[C@@H](C)CN1Cc1ccc(F)cc1C(F)F]

To a DMF (2 mL) solution of 8-((2S,5R)-2,5-dimethylpiperazin-1-yl)-5-methyl-6-oxo-5,6-dihydro-1,5-naphthyridine-2-carbonitrile (30 mg, 0.081 mmol) was added 2-(difluoromethyl)-4-fluorobenzaldehyde (16.86 mg, 0.097 mmol). The solution was stirred at room temperature for 1 hour. Sodium cyanoborohydride (15.22 mg, 0.242 mmol) was added and the reaction mixture was stirred at room temperature overnight. LC/MS analysis indicated the reaction was complete. The crude material was purified via preparative LC/MS with the following conditions: Column: XBridge C18, 200 mm×19 mm, 5 μm particles; Mobile Phase A: 5:95 acetonitrile:water with 10 mM ammonium acetate; Mobile Phase B: 95:5 acetonitrile:water with 10 mM ammonium acetate; Gradient: a 0 minute hold at 31% B, 31-71% B over 25 minutes, then a 5 minute hold at 100% B; Flow Rate: 20 mL/min; Column Temperature: 25° C. Fraction collection was triggered by MS and UV signals. Fractions containing the product were combined and dried via centrifugal evaporation. The yield of the product was 13.0 mg, and the estimated purity by LCMS analysis was 100%. Analytical LC/MS was used to determine the final purity. Injection 1 conditions: Column: Waters XBridge C18, 2.1 mm×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0% B to 100% B over 3 min, then a 0.50 min hold at 100% B; Flow: 1 mL/min; Detection: MS and UV (220 nm). Injection 1 results: Purity: 100.0%; Observed Mass: 456.08; Retention Time: 1.39 min. Injection 2 conditions: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm). Injection 2 results: Purity: 100.0%; Observed Mass: 456.07; Retention Time: 2.22 min. % B over 3 min, then a 0.50 min hold at 100% B; Flow: 1 mL/min; Detection: MS and UV (220 nm). Injection 2 results: Purity: 100.0%; Observed Mass: 456.07; Retention Time: 2.22 min.

### Method of Synthesizing DGKi Compound 10

### 8-[(2S,5R)-4-[(4-fluorophenyl)(4-methylphenyl)methyl]-2,5-dimethylpiperazin-1-yl]-5-methyl-6-oxo-5,6-dihydro-1,5-naphthyridine-2-carbonitrile

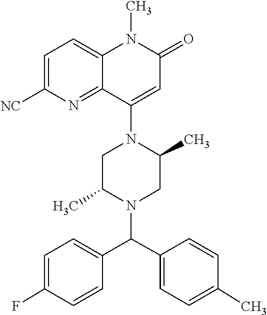 [SMILES: Cc1ccc(C(c2ccc(F)cc2)N2C[C@H](C)N(c3cc(=O)n(C)c4ccc(C#N)nc34)C[C@H]2C)cc1]

To the mixture of 8-((2S,5R)-2,5-dimethylpiperazin-1-yl)-5-methyl-6-oxo-5,6-dihydro-1,5-naphthyridine-2-carbonitrile, TFA (68.6 mg, 60% wt., 0.1 mmol), (cyanomethyl)trimethylphosphonium iodide (48.6 mg, 0.200 mmol) and (4-fluorophenyl)(p-tolyl)methanol (26.0 mg, 0.120 mmol) in acetonitrile (0.3 mL) was added Hunig's base (0.105 mL, 0.600 mmol). The reaction mixture was stirred at 110° C. for 2 hours, followed by a second addition of (cyanomethyl)trimethylphosphonium iodide (48.6 mg, 0.200 mmol), (4-fluorophenyl)(p-tolyl)methanol (26.0 mg, 0.120 mmol) and Hunig's base (0.058 mL, 0.300 mmol). The reaction mixture was stirred at 110° C. for another 2 hours. The crude reaction mixture was injected directly on 12 g Si-RediSep Rf for flash chromatography by 20-100% ethyl acetate in hexanes. Product containing fractions were combined and dried by vacuum. The resultant material was further purified via preparative LC/MS with the following conditions: Column: XBridge C18, 200 mm×19 mm, 5 μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0 minute hold at 20% B, 20-60% B over 25 minutes, then a 5 minute hold at 100% B; Flow Rate: 20 mL/min; Column Temperature: 25° C. Fraction collection was triggered by MS and UV signals. Fractions containing the product were combined and dried via centrifugal evaporation. The yield of the diastereomeric product TFA salt was 47.1 mg.

The diasteromeric product was resolved into two diastereomers by using SFC-chiral chromatography with the following conditions: Column: Chiral AD, 30×250 mm, 5 micron particles; Mobile Phase: 80% CO₂/20% IPA w/0.1% DEA; Flow Rate: 100 mL/min; Column Temperature: 25° C. The title compound was collected as the 2^{nd} eluent peak, >91% de. Calculated molecular weight 495.602. Analytical LC/MS was used to determine the final purity. Injection 1 conditions: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm). Injection 1 results: Purity: 97.6%; Observed Mass: 496.26; Retention Time: 2.52 min. Injection 2 conditions: Column: Waters XBridge C18, 2.1 mm×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0% B to 100% B over 3 min, then a 0.50 min hold at 100% B; Flow: 1 mL/min; Detection: MS and UV (220 nm). Injection 2 results: Purity: 98.2%; Observed Mass: 496.28; Retention Time: 1.73 min.

### Method of Synthesizing DGKi Compound 11

### 8-[(2S,5R)-4-[1-(2,6-difluorophenyl)ethyl]-2,5-dimethylpiperazin-1-yl]-5-methyl-6-oxo-5,6-dihydro-1,5-naphthyridine-2-carbonitrile

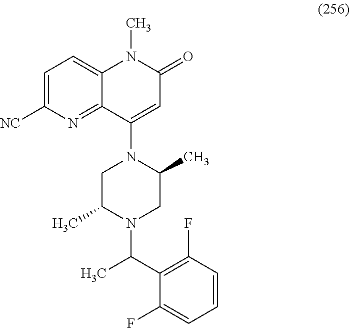 [SMILES: CC(c1c(F)cccc1F)N1C[C@H](C)N(c2cc(=O)n(C)c3ccc(C#N)nc23)C[C@H]1C]

To a mixture of 2-(1-bromoethyl)-1,3-difluorobenzene (15.12 mg, 0.065 mmol) and 5-methyl-6-oxo-8-(piperazin-1-yl)-5,6-dihydro-1,5-naphthyridine-2,7-dicarbonitrile, TFA (34.0 mg, 60% wt., 0.05 mmol) in acetonitrile (0.3 mL) was added Hunig's base (0.052 mL, 0.300 mmol). The mixture was stirred at 55° C. for 2 hours. LCMS indicated complete conversion to product. The crude material was purified via preparative LC/MS with the following conditions: Column: XBridge C18, 200 mm×19 mm, 5 μm particles; Mobile Phase A: 5:95 acetonitrile:water with 10 mM ammonium acetate; Mobile Phase B: 95:5 acetonitrile:water with 10 mM ammonium acetate; Gradient: a 0 minute hold at 37% B, 37-77% B over 20 minutes, then a 5 minute hold at 100% B; Flow Rate: 20 mL/min; Column Temperature: 25° C. Fraction collection was triggered by MS and UV signals. Fractions containing the product were combined and dried via centrifugal evaporation. The yield of the product was 12.1 mg. Calculated molecular weight 437.495. Analytical LC/MS was used to determine the final purity. Injection 1 conditions: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm). Injection 1 results: Purity: 100.0%; Observed Mass: 438.14; Retention Time: 2.36 min. Injection 2 conditions: Column: Waters XBridge C18, 2.1 mm×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0% B to 100% B over 3 min, then a 0.50 min hold at 100% B; Flow: 1 mL/min; Detection: MS and UV (220 nm). Injection 2 results: Purity: 100.0%; Observed Mass: 438.14; Retention Time: 1.2 min.

### Method of Synthesizing DGKi Compounds 12-14

### 8-((2S,5R)-4-(1-(2,4-difluorophenyl)propyl)-2,5-dimethylpiperazin-1-yl)-5-methyl-6-oxo-5,6-dihydro-1,5-naphthyridine-2-carbonitrile

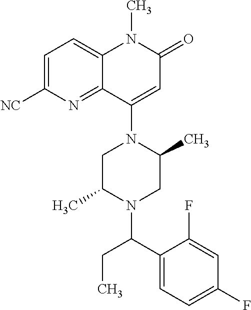 [SMILES: CCC(c1ccc(F)cc1F)N1C[C@H](C)N(c2cc(=O)n(C)c3ccc(C#N)nc23)C[C@H]1C]

To a mixture of 8-((2S,5R)-2,5-dimethylpiperazin-1-yl)-5-methyl-6-oxo-5,6-dihydro-1,5-naphthyridine-2-carbonitrile (29.7 mg, 0.1 mmol) and 1-(1-bromopropyl)-2,4-difluorobenzene (25.9 mg, 0.110 mmol) in acetonitrile (0.3 mL) was added Hunig's base (87 μL, 0.500 mmol). The mixture was stirred on hot plate at 55° C. for 16 hours. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0 minute hold at 3% B, 3-43% B over 25 minutes, then a 5 minute hold at 100% B; Flow Rate: 20 mL/min; Column Temperature: 25° C. Fraction collection was triggered by MS and UV signals. Fractions containing the product were combined and dried via centrifugal evaporation. Stereochemistry: diasteromeric mixture.

The diastereomeric mixture of the synthesis of DGKi Compound 12 was further separated to resolve two homochiral diastereomers by using SFC-chiral chromatography with the following conditions: Column: Chiral OD, 30×250 mm. 5 micron particles; Mobile Phase: 15% IPA/85% CO₂ w/0.1% DEA; Flow Rate: 100 m/min; Detector Wavelength: 220 nm.

DGKi Compound 13 (Isomer 1) was collected as the first eluent peak in 95% de. Stereochemistry: Homochiral.

DGKi Compound 14 (Isomer 2) was collected as the second eluent peak in 95% de. Stereochemistry: Homochiral.

### Method of Synthesizing DGKi Compound 15

### 8-(4-(bis(4-fluorophenyl)methyl)piperazin-1-yl)-5-methyl-7-nitro-6-oxo-5,6-dihydro-1,5-naphthyridine-2-carbonitrile

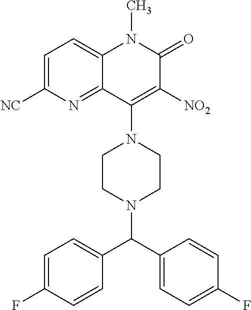 [SMILES: Cn1c(=O)c([N+](=O)[O-])c(N2CCN(C(c3ccc(F)cc3)c3ccc(F)cc3)CC2)c2nc(C#N)ccc21]

DMF was sparged with nitrogen for 1 hour. In a 1 dram vial was charged with zinc (0.95 mg, 0.015 mmol), bromo(tri-tert-butylphosphine)palladium(I) dimer (9.96 mg, 0.013 mmol) and 4-(4-(bis(4-fluorophenyl)methyl)piperazin-1-yl)-6-bromo-1-methyl-3-nitro-1,5-naphthyridin-2(1H)-one (21.38 mg, 0.037 mmol). The sparged DMF (0.3 mL) was added and the mixture was capped under nitrogen and immersed in a 50° C. oil bath for 15 minutes. Dicyanozinc (2.86 mg, 0.024 mmol) was added. The reaction mixture was capped under nitrogen and immersed in 50° C. oil bath for 3 hours. LC/MS analysis indicated the reaction was complete. The crude material was purified via preparative LC/MS with the following conditions: Column: XBridge C18, 19×200 mm, 5 μm particles; Mobile Phase A: 5:95 acetonitrile:water with 10 mM ammonium acetate; Mobile Phase B: 95:5 acetonitrile:water with 10 mM ammonium acetate; Gradient: 50-90% B over 15 minutes, then a 5 minute hold at 100% B; Flow: 20 mL/minute. Fractions containing the product were combined and dried via centrifugal evaporation. The title compound (11.4 mg) was isolated in 59.7% yield.

Alternative synthesis: A DMF (6 mL) solution of 8-chloro-5-methyl-7-nitro-6-oxo-5,6-dihydro-1,5-naphthyridine-2-carbonitrile (750 mg, 2.83 mmol) was combined with 1-(bis(4-fluorophenyl)methyl)piperazine (899 mg, 3.12 mmol)) followed by the addition of Hunig's Base (0.990 mL, 5.67 mmol). The reaction mixture was stirred at room temperature overnight. LC/MS analysis indicated the reaction was completed. The crude material was filtered and purified by preparative HPLC employing aqueous acetonitrile with ammonium acetate as the buffer to afford 1.02 g of yellow solid. Two analytical LC/MS injections were used to determine the final purity. Injection 1 conditions: Column: Waters Acquity UPLC BEH C18, 2.1×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75 minute hold at 100% B; Flow: 1.0 mL/minute; Detection: UV at 220 nm. Injection 2 conditions: Column: Waters Acquity UPLC BEH C18, 2.1×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0-100% B over 3 minutes, then a 0.75 minute hold at 100% B; Flow: 1.0 mL/minute; Detection: UV at 220 nm. Injection 1 results: Purity: 100%; Observed Mass: 517.0; Retention Time: 2.4 minutes. Injection 2 results: Purity: 98.4%; Observed Mass: 517.0; Retention Time: 1.7 minutes. ¹H NMR (500 MHz, chloroform-d) δ 7.88 (d, J=8.7 Hz, 1H), 7.76 (d, J=8.9 Hz, 1H), 7.40 (dd, J=8.5, 5.5 Hz, 4H), 7.02 (t, J=8.7 Hz, 4H), 4.34 (s, 1H), 3.68 (s, 3H), 3.62-3.55 (m, 4H), 2.64 (br s, 4H). ¹³C NMR (126 MHz, chloroform-d) δ 163.0, 161.0, 155.4, 147.0, 138.0, 137.7, 137.7, 135.9, 132.4, 129.5, 129.2, 129.2, 126.0, 123.1, 116.5, 115.8, 115.6, 74.3, 51.6, 51.2, 29.7.

### Method of Synthesizing DGKi Compound 16

### 8-[(2S,5R)-4-[bis(4-methylphenyl)methyl]-2,5-dimethylpiperazin-1-yl]-5-methyl-6-oxo-5,6-dihydro-1,5-naphthyridine-2-carbonitrile

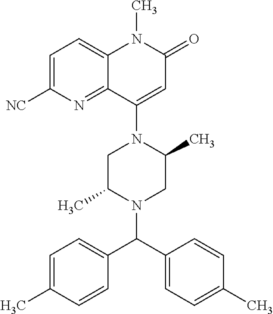

To the mixture of (cyanomethyl)trimethylphosphonium iodide (46.2 mg, 0.19 mmol), di-p-tolylmethanol (23.46 mg, 0.108 mmol), and 8-((2S,5R)-2,5-dimethylpiperazin-1-yl)-5-methyl-6-oxo-5,6-dihydro-1,5-naphthyridine-2-carbonitrile, TFA (72.4 mg, 54% wt, 0.095 mmol) in acetonitrile (0.3 mL) was added Hunig's base (0.10 mL, 0.57 mmol). The reaction mixture was stirred at 110° C. for 2 hours. The crude material was purified via preparative LC/MS with the following conditions: Column: XBridge C18, 200 mm×19 mm, 5 μm particles; Mobile Phase A: 5:95 acetonitrile:water with 10 mM ammonium acetate; Mobile Phase B: 95:5 acetonitrile:water with 10 mM ammonium acetate; Gradient: a 0 minute hold at 55% B, 55-95% B over 20 minutes, then a 4 minute hold at 100% B; Flow Rate: 20 mL/min; Column Temperature: 25° C. Fraction collection was triggered by MS and UV signals. Fractions containing the product were combined and dried via centrifugal evaporation. The yield of the product was 23.4 mg. Calculated molecular weight 491.639. Analytical LC/MS was used to determine the final purity. Injection 1 conditions: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.75 min hold at 100% B; Flow: 1 mL/min; Detection: MS and UV (220 nm). Injection 1 results: Purity: 100.0%; Observed Mass: 492.21; Retention Time: 2.77 min. Injection 2 conditions: Column: Waters XBridge C18, 2.1 mm×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0% B to 100% B over 3 min, then a 0.75 min hold at 100% B; Flow: 1 mL/min; Detection: MS and UV (220 nm). Injection 2 results: Purity: 100.0%; Observed Mass: 492.2; Retention Time: 1.71 min. ¹H NMR (400 MHz, DMSO-d₆) δ ppm 8.15 (d, J=8.5 Hz, 1H), 8.04-8.09 (m, 1H), 7.81 (s, 4H), 7.57-7.63 (m, 2H), 7.12-7.19 (m, 2H), 6.00 (s, 1H), 4.82 (s, 1H), 4.52-4.63 (m, 1H), 3.64-3.76 (m, 1H), 3.51-3.58 (m, 4H), 2.99-3.10 (m, 1H), 2.86 (br d, J=8.5 Hz, 1H), 2.28-2.37 (m, 1H), 1.31 (d, J=6.5 Hz, 3H), 1.07 (d, J=6.5 Hz, 3H). ¹³C NMR (100.66 MHz, DMSO-d₆) δ ppm 162.4, 160.9, 159.9, 153.5, 148.0, 138.7, 138.6, 135.0, 132.6, 129.3 (d, J=8.0 Hz), 128.8 (d, J=10.0 Hz), 124.0, 122.8, 118.6, 117.5, 115.6, 115.4, 109.8, 104.8, 69.0, 51.8, 49.4, 48.9, 47.2, 28.6, 13.4, 7.4.

Reference: PCT/US2020/048070

### DGKi Compounds 17 and 18

### 4-((2S,5R)-2,5-Diethyl-4-(1-(4-(trifluoromethyl)phenyl)propyl)piperazin-1-yl)-1-methyl-2-oxo-1,2-dihydropyrido[3,2-d]pyrimidine-6-carbonitrile

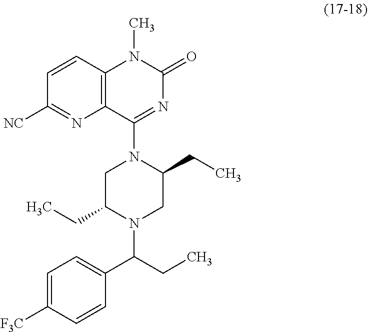 [SMILES: CCC(c1ccc(C(F)(F)F)cc1)N1C[C@H](CC)N(c2nc(=O)n(C)c3ccc(C#N)nc23)C[C@H]1CC]

To a stirred solution of 4-((2S,5R)-2,5-diethylpiperazin-1-yl)-1-methyl-2-oxo-1,2-dihydropyrido[3,2-d]pyrimidine-6-carbonitrile, TFA (0.12 g, 0.27 mmol) in acetonitrile (10 mL) were added DIPEA (0.14 mL, 0.82 mmol), 1-(1-chloropropyl)-4-(trifluoromethyl)benzene (0.12 g, 0.55 mmol), and sodium iodide (0.04 g, 0.27 mmol). The reaction mixture was heated at 85° C. for 16 h. The reaction mixture was cooled to room temperature and the solvent was removed under reduced pressure to yield the crude product, which was purified by preparative HPLC [HPLC Method: Column: Sunfire C18, 150×19 mm ID, 5 μm; Mobile Phase A: 10 mM ammonium acetate in water; Mobile Phase B: acetonitrile; Gradient: 0-100% B over 18 minutes, then a 5 minute hold at 100% B; Flow: 17 mL/min]. The fractions were concentrated under reduced pressure and lyophilized from EtOH/H₂O (1:5) to yield Compounds 17 and 18.

Compound 17: (10 mg, 7% yield); LCMS: m/z=513.3 (M+H); rt 2.52 min; (LCMS method: Column: XBridge BEH XP C18 (50×2.1) mm, 2.5 μm Mobile phase A: 95% water: 5% acetonitrile; 10 mM ammonium formate; Mobile phase B: 5% Water: 95% acetonitrile; 10 mM ammonium formate; Flow: 1.1 mL/min; Temp: 50° C.; Time (min): 0-4; % B: 0-100; ¹H NMR (400 MHz, DMSO-d₆) δ 8.24 (d, J=6.6 Hz, 1H), 7.98 (d, J=9.0 Hz, 1H), 7.73 (d, J=8.1 Hz, 2H), 7.56 (d, J=7.1 Hz, 2H), 5.83-5.48 (m, 1H), 4.98-4.86 (m, 1H), 3.64 (br. s., 1H), 3.43 (s, 3H), 3.08 (d, J=9.8 Hz, 1H), 2.93-2.82 (m, 2H), 2.42-2.26 (m, 1H), 2.13-2.08 (m, 1H), 1.98-1.82 (m, 3H), 1.66-1.54 (m, 1H), 1.44-1.31 (m, 1H), 0.98-0.91 (br. s., 3H), 0.69-0.53 (m, 6H).

Compound 18: (3 mg, 2% yield); LCMS: m/z=513.3 (M+H); rt 2.54 min; (LCMS method: Column: XBridge BEH XP C18 (50×2.1) mm, 2.5 μm Mobile phase A: 95% water: 5% acetonitrile; 10 mM ammonium formate; Mobile phase B: 5% Water: 95% acetonitrile; 10 mM ammonium formate; Flow: 1.1 mL/min; Temp: 50° C.; Time (min): 0-4; % B: 0-100; ¹H NMR (400 MHz, DMSO-d₆) δ 8.28-8.19 (m, 1H), 8.01-7.95 (m, 1H), 7.72 (d, J=7.8 Hz, 2H), 7.58 (d, J=8.6 Hz, 2H), 6.06-5.28 (m, 1H), 5.08-4.76 (m, 1H), 3.64-3.50 (m, 2H), 3.43 (s, 3H), 3.16-3.08 (m, 1H), 2.25-2.14 (m, 2H), 2.00-1.83 (m, 3H), 1.57-1.53 (m, 3H), 1.03-0.89 (m, 3H), 0.65-0.54 (m, 6H).

### DGKi Compounds 19 and 20

### 4-((2S,5R)-5-Ethyl-2-methyl-4-(1-(4-(trifluoromethyl)phenyl)ethyl)piperazin-1-yl)-1-methyl-2-oxo-1,2-dihydropyrido[3,2-d]pyrimidine-6-carbonitrile

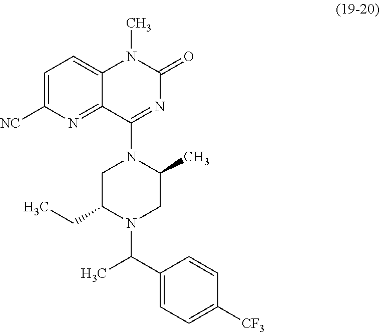 [SMILES: CC[C@@H]1CN(c2nc(=O)n(C)c3ccc(C#N)nc23)[C@@H](C)CN1C(C)c1ccc(C(F)(F)F)cc1]

To a stirred solution of 4-((2S,5R)-5-ethyl-2-methylpiperazin-1-yl)-1-methyl-2-oxo-1,2-dihydropyrido[3,2-d]pyrimidine-6-carbonitrile, TFA (70 mg, 0.22 mmol) in acetonitrile (2 mL) at room temperature were added DIPEA (0.12 mL, 0.67 mmol), 1-(1-chloroethyl)-4-(trifluoromethyl)benzene (93 mg, 0.45 mmol), sodium iodide (33.6 mg, 0.22 mmol) and heated at 85° C. for 16 h. The reaction mixture cooled to room temperature and the solvent was removed under reduced pressure, the residue was dissolved in ethyl acetate (100 mL). The organic layer was washed with brine, dried over Na₂SO₄ and concentrated under reduced pressure to yield the crude product, which was purified by preparative HPLC [HPLC Method: Column: Sunfire C18 (150 mm×19.2 mm ID, 5 μm), Mobile phase A=10 mM ammonium acetate in water, Mobile phase B=acetonitrile, Flow: 19 mL/min], fractions were concentrated under reduced pressure, diluted with EtOH/H₂O (1:5), and lyophilized to yield Compounds 19 and 20.

Compound 19: (9 mg, 8% yield); LCMS: m/z=485.1 (M+H); rt 2.34 min; (LCMS method: Column: XBridge BEH XP C18 (50×2.1 mm), 2.5 μm; Mobile phase A: 95% water: 5% acetonitrile; 10 mM ammonium acetate; Mobile phase B: 5% Water: 95% acetonitrile; 10 mM ammonium acetate; Flow: 1.1 mL/min; Temp: 50° C.; Time (min): 0-3; % B: 0-100. ¹H NMR (400 MHz, DMSO-d₆) δ ppm 8.32-8.17 (m, 1H), 8.05-7.94 (m, 1H), 7.76-7.66 (m, 2H), 7.66-7.55 (m, 2H), 6.11-5.42 (m, 1H), 5.10-4.79 (m, 1H), 3.78-3.59 (m, 2H), 3.44 (s, 3H), 3.17-3.05 (m, 1H), 2.64-2.55 (m, 1H), 2.26-2.09 (m, 1H), 1.65-1.34 (m, 3H), 1.31-1.16 (m, 5H), 1.01 (br t, J=7.1 Hz, 3H).

Compound 20: (9 mg, 8% yield); LCMS: m/z=485.1 (M+H); rt 2.29 min; (LCMS Method: Column: XBridge BEH XP C18 (50×2.1 mm), 2.5 μm; Mobile phase A: 95% water: 5% acetonitrile; 10 mM ammonium acetate; Mobile phase B: 5% Water: 95% acetonitrile; 10 mM ammonium acetate; Flow: 1.1 mL/min; Temp: 50° C.; Time (min): 0-3; % B: 0-100%). ¹H NMR (400 MHz, DMSO-d₆) δ ppm 8.24 (br d, J=8.6 Hz, 1H), 7.99 (d, J=9.0 Hz, 1H), 7.73 (d, J=8.3 Hz, 2H), 7.61 (br d, J=8.3 Hz, 2H), 5.87-5.63 (m, 1H), 5.10-4.79 (m, 1H), 3.90-3.80 (m, 1H), 3.44 (s, 3H), 3.46-3.15 (m, 1H), 2.89-2.73 (m, 2H), 2.41-2.34 (m, 1H), 1.63-1.34 (m, 5H), 1.29 (br d, J=6.1 Hz, 3H), 0.79-0.64 (m, 3H).

### DGKi Compounds 21 and 22

### 4-((2S,5R)-5-Ethyl-4-((4-fluorophenyl)(5-(trifluoromethyl)pyridin-2-yl)methyl)-2-methylpiperazin-1-yl)-1-methyl-2-oxo-1,2-dihydropyrido[3,2-d]pyrimidine-6-carbonitrile

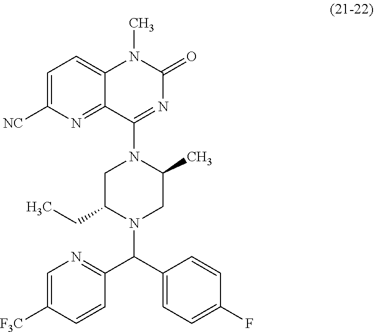 [SMILES: CC[C@@H]1CN(c2nc(=O)n(C)c3ccc(C#N)nc23)[C@@H](C)CN1C(c1ccc(F)cc1)c1ccc(C(F)(F)F)cn1]

To a stirred solution of 4-((2S,5R)-5-ethyl-2-methylpiperazin-1-yl)-1-methyl-2-oxo-1,2-dihydropyrido[3,2-d]pyrimidine-6-carbonitrile, TFA (0.5 g, 1.17 mmol) in acetonitrile (10 mL) was added DIPEA (1.02 mL, 5.86 mmol), followed by 2-(bromo(4-fluorophenyl)methyl)-5-(trifluoromethyl)pyridine (0.78 mg, 2.35 mmol). The reaction mixture was heated at 80° C. for 3 h. The reaction mixture was cooled to room temperature and the solvent was removed under reduced pressure to yield the crude product, which was purified by preparative HPLC (HPLC Method: Column: INERTSIL ODS 21.2×250 mm, 5 μm; Mobile Phase A: 0.1% TFA in water; Mobile Phase B: acetonitrile; Gradient: 30-80% B over 14 minutes, then a 5 minute hold at 100% B; Flow: 17 m/min), fractions were concentrated under reduced pressure and lyophilized from (EtOH/H₂O, 1:5) to yield Compound 21 and Compound 22.

Compound 21: 140 mg, 21% yield; LCMS: m/z=566.2 (M+H); rt 3.26 min; (LCMS method: Column: Column-Kinetex XB-C18 (75×3 mm-2.6 μm), Mobile phase A: 98% water: 2% acetonitrile; 10 mM ammonium formate; Mobile phase B: 2% Water: 98% acetonitrile; 10 mM ammonium formate; Flow: 1.0 mL/min; Temp: 50° C.; Time (min): 0-4; % B: 0-100%). ¹H NMR (400 MHz, DMSO-d₆) δ ppm 8.83 (br s, 1H), 8.19-8.31 (m, 2H), 7.95-8.12 (m, 2H), 7.53-7.63 (m, 2H), 7.12-7.26 (m, 2H), 5.41-6.26 (m, 1H), 4.79-5.20 (m, 2H), 3.60-3.74 (m, 1H), 3.44 (s, 3H), 2.73-2.87 (m, 1H), 2.22-2.42 (m, 2H), 1.40-1.68 (m, 5H), 0.53-0.71 (m, 3H).

Compound 22: 155 mg, 23% yield; LCMS: m/z=566.2 (M+H); rt 3.25 min; (LCMS method: Column: Column-Kinetex XB-C18 (75×3 mm-2.6 μm), Mobile phase A: 98% water: 2% acetonitrile; 10 mM ammonium formate; Mobile phase B: 2% Water: 98% acetonitrile; 10 mM ammonium formate; Flow: 1.0 mL/min; Temp: 50° C.; Time (min): 0-4; % B: 0-100%). ¹H NMR (400 MHz, DMSO-d₆) δ ppm 8.92 (s, 1H), 8.17-8.27 (m, 2H), 7.90-8.02 (m, 2H), 7.60-7.67 (m, 2H), 7.14-7.22 (m, 2H), 5.52-6.07 (m, 1H), 4.87-5.08 (m, 2H), 3.39-3.71 (m, 4H), 2.69-2.78 (m, 1H), 2.37-2.45 (m, 1H), 1.37-1.69 (m, 5H), 0.58-0.77 (m, 3H).

### DGKi Compounds 23-24

### (4-((2S,5R)-4-((4-chlorophenyl)(pyridin-2-yl)methyl)-5-ethyl-2-methylpiperazin-1-yl)-1-methyl-2-oxo-1,2-dihydropyrido[3,2-d]pyrimidine-6-carbonitrile

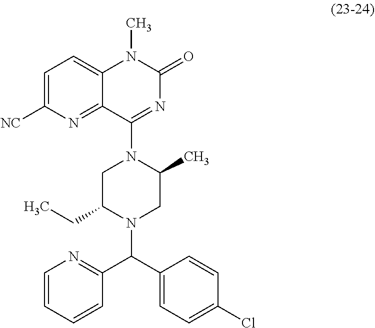 [SMILES: CC[C@@H]1CN(c2nc(=O)n(C)c3ccc(C#N)nc23)[C@@H](C)CN1C(c1ccc(Cl)cc1)c1ccccn1]

To a stirred solution of 4-((2S,5R)-5-ethyl-2-methylpiperazin-1-yl)-1-methyl-2-oxo-1,2-dihydropyrido[3,2-d]pyrimidine-6-carbonitrile (100 mg, 0.32 mmol) in acetonitrile (5 mL) was added DIPEA (0.3 mL, 1.60 mmol), followed by 2-(bromo(4-chlorophenyl)methyl)pyridine (181 mg, 0.64 mmol). The reaction mixture was heated at 80° C. for 3 h. The reaction mixture was cooled to room temperature and the solvent was removed under reduced pressure to yield the crude product, which was purified by preparative HPLC (HPLC Method: Column: Cellulose-5 (250*20 ID) 5 micron; Mobile Phase A: 0.1% DEA in IPA; Mobile Phase B: 0.1% DEA in ACN; Gradient: 90% of B, then a 5 minute hold at 100% B; Flow: 18 mL/min), fractions were concentrated under reduced pressure and lyophilized from (EtOH/H₂O, 1:5) to yield Compound 23 and Compound 24.

Compound 23: 24 mg, 14% yield; LCMS: m/z=514.2 (M+H); rt 2.94 min; (LCMS method: Column: Column-Kinetex XB-C18 (75×3 mm-2.6 μm), Mobile phase A: 98% water: 2% acetonitrile; 10 mM ammonium formate; Mobile phase B: 2% Water: 98% acetonitrile; 10 mM ammonium formate; Flow: 1.0 mL/min; Temp: 50° C.; Time (min): 0-4; % B: 0-100%). ¹H NMR (400 MHz, DMSO-d₆): δ ppm 8.52 (d, J=4.5 Hz, 1H), 8.23 (d, J=9.0 Hz, 1H), 7.96-8.02 (m, 1H), 7.75-7.81 (m, 1H), 7.59-7.68 (m, 3H), 7.39 (d, J=8.5 Hz, 2H), 7.22-7.29 (m, 1H), 5.54-5.95 (m, 1H), 4.81-5.07 (m, 2H), 3.39-3.68 (m, 5H), 2.69-2.76 (m, 1H), 2.35-2.44 (m, 1H), 1.37-1.67 (m, 5H), 0.58-0.67 (m, 3H).

Compound 24: 22 mg, 13% yield; LCMS: m/z=514.2 (M+H); rt 2.94 min; (LCMS method: Column: Column-Kinetex XB-C18 (75×3 mm-2.6 μm), Mobile phase A: 98% water: 2% acetonitrile; 10 mM ammonium formate; Mobile phase B: 2% Water: 98% acetonitrile; 10 mM ammonium formate; Flow: 1.0 mL/min; Temp: 50° C.; Time (min): 0-4; % B: 0-100%). ¹H NMR (400 MHz, DMSO-d₆): δ ppm 8.41-8.45 (m, 1H), 8.23 (d, J=9.0 Hz, 1H), 7.96-8.02 (m, 1H), 7.78-7.85 (m, 2H), 7.53-7.61 (m, 2H), 7.40 (d, J=8.5 Hz, 2H), 7.20-7.26 (m, 1H), 5.52-5.97 (m, 1H), 4.87-5.04 (m, 1H), 4.78-4.86 (m, 1H), 3.37-3.71 (m, 4H), 2.72-2.78 (m, 1H), 2.54-2.63 (m, 1H), 2.35-2.46 (m, 1H), 1.40-1.64 (m, 5H), 0.58-0.70 (m, 3H).

### DGKi Compounds 25 and 26

### 4-((2S,5R)-4-((3-Cyclopropyl-1,2,4-oxadiazol-5-yl)(4-fluorophenyl)methyl)-2,5-dimethylpiperazin-1-yl)-1-methyl-2-oxo-1,2-dihydropyrido[3,2-d]pyrimidine-6-carbonitrile

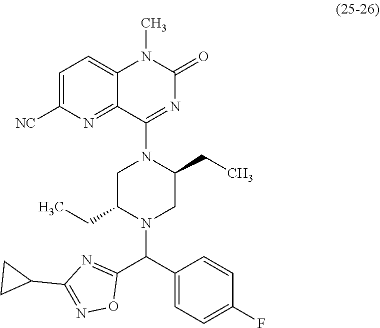 [SMILES: CC[C@H]1CN(C(c2ccc(F)cc2)c2nc(C3CC3)no2)[C@H](CC)CN1c1nc(=O)n(C)c2ccc(C#N)nc12]

To a stirred solution of 2-((2R,5S)-4-(6-cyano-1-methyl-2-oxo-1,2-dihydropyrido[3,2-d]pyrimidin-4-yl)-2,5-diethylpiperazin-1-yl)-2-(4-fluorophenyl)acetic acid, (0.045 g, 0.09 mmol), N-hydroxycyclopropanecarboximidamide (9.4 mg, 0.09 mmol) in DMF (2 mL), BOP (0.01 g, 0.23 mmol) and triethylamine (0.04 mL, 0.23 mmol) were added at room temperature. After 2 hours, the reaction mixture was heated at 110° C. for 3 h. The reaction mixture was cooled to room temperature and evaporated under reduced pressure to yield crude product, which was purified via preparative HPLC. Chiral Separation Method: Column: DAD-1-Cellulose-2 (250×4.6 mm), 5 micron. Mobile Phase: 0.1% DEA in acetonitrile, Flow: 2.0 mL\min.

Compound 25: (1.9 mg, 6% yield): LCMS: m/z, 543.3 (M+H); rt 2.21 min; LCMS method: Column: XBridge BEH XP C18 (50×2.1) mm, 2.5 μm; Mobile phase A: 95% water: 5% acetonitrile; 10 mM ammonium acetate; Mobile phase B: 5% water: 95% acetonitrile; 10 mM ammonium acetate; Flow: 1.1 mL/min; Temp: 50° C.; Time (min) Time (min): 0-3; % B: 0-100%). ¹H NMR (400 MHz, DMSO-d₆) δ ppm 8.29-8.16 (m, 1H), 8.06-7.92 (m, 1H), 7.75-7.58 (m, 2H), 7.26 (m, 2H), 6.01-5.32 (m, 1H), 5.28 (br s, 1H), 5.00-4.79 (m, 1H), 3.66-3.56 (m, 1H), 3.43 (s, 3H), 2.65-2.57 (m, 1H), 2.44-2.34 (m, 2H), 2.18-2.00 (m, 1H), 1.95-1.74 (m, 2H), 1.68-1.34 (m, 2H), 1.15-1.02 (m, 2H), 0.93-0.83 (m, 2H), 0.81-0.62 (m, 6H).

Compound 26: (1.0 mg, 3% yield): LCMS: m/z, 543.3 (M+H); rt 2.20 min; LCMS method: Column: XBridge BEH XP C18 (50×2.1) mm, 2.5 μm; Mobile phase A: 95% water: 5% acetonitrile; 10 mM ammonium acetate; Mobile phase B: 5% water: 95% acetonitrile; 10 mM ammonium acetate; Flow: 1.1 mL/min; Temp: 50° C.; Time (min) Time (min): 0-3; % B: 0-100%). ¹H NMR (400 MHz, DMSO-d₆) δ ppm 8.23 (d, J=8.8 Hz, 1H), 8.06-7.91 (m, 1H), 7.62 (dd, J=6.2, 7.5 Hz, 2H), 7.26 (t, J=8.8 Hz, 2H), 5.92-5.31 (m, 1H), 5.29 (s, 1H), 4.96-4.78 (m, 1H), 3.60-3.50 (m, 1H), 3.43 (s, 3H), 3.25-3.10 (m, 1H), 2.97-2.75 (m, 2H), 2.27-1.65 (m, 3H), 1.49-1.24 (m, 2H), 1.11-0.97 (m, 2H), 0.94-0.75 (m, 5H), 0.74-0.50 (m, 3H).

### DGKi Compounds 27 and 28

### 4-((2S,5R)-4-((4-fluorophenyl)(5-(trifluoromethyl)pyridin-2-yl)methyl)-2,5-dimethylpiperazin-1-yl)-1-methyl-2-oxo-1,2-dihydropyrido[3,2-d]pyrimidine-6-carbonitrile

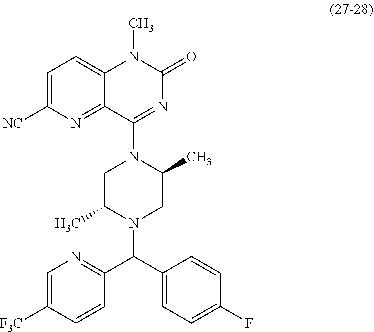 [SMILES: C[C@@H]1CN(c2nc(=O)n(C)c3ccc(C#N)nc23)[C@@H](C)CN1C(c1ccc(F)cc1)c1ccc(C(F)(F)F)cn1]

To a stirred solution of 4-((2S,5R)-2,5-dimethylpiperazin-1-yl)-1-methyl-2-oxo-1,2-dihydropyrido[3,2-d]pyrimidine-6-carbonitrile (1 g, 3.35 mmol) in acetonitrile (10 mL) was added DIPEA (5.9 mL, 33.5 mmol), followed by 2-(bromo(4-fluorophenyl)methyl)-5-(trifluoromethyl)pyridine (2.24 g, 6.70 mmol). The reaction mixture was heated at 80° C. for 4 h. The reaction mixture was cooled to room temperature and the solvent was removed under reduced pressure to yield the crude product, which was purified by preparative HPLC (HPLC Method: Column: Sunfire C18, 150×19 mm ID, 5 μm; Mobile Phase A: 0.1% TFA in water; Mobile Phase B: Acetonitrile:MeOH (1:1); Gradient: 50-100% B over 20 minutes, then a 5 minute hold at 100% B; Flow: 19 mL/min), fractions were concentrated under reduced pressure and lyophilized from (EtOH/H₂O, 1:5) to yield Compound 27 and Compound 28.

Compound 27: 110 mg, 6% yield; LCMS: m/z=552.2 (M+H); rt 3.09 min; (LCMS method: Column: Column-Kinetex XB-C18 (75×3 mm-2.6 μm), Mobile phase A: 98% water: 2% acetonitrile; 10 mM ammonium formate; Mobile phase B: 2% Water: 98% acetonitrile; 10 mM ammonium formate; Flow: 1.0 mL/min; Temp: 50° C.; Time (min): 0-4; % B: 20-100%). ¹H NMR (400 MHz, DMSO-d₆) δ ppm 8.83 (s, 1H), 8.22 (d, J=9.0 Hz, 2H), 8.11-7.95 (m, 2H), 7.71-7.58 (m, 2H), 7.25-7.13 (m, 2H), 5.76-5.44 (m, 1H), 5.13-4.67 (m, 2H), 3.86-3.49 (m, 1H), 3.44 (s, 3H), 3.19-3.08 (m, 1H), 2.84 (dd, J=3.8, 12.3 Hz, 1H), 2.38-2.26 (m, 1H), 1.67-1.39 (m, 3H), 1.11-0.86 (m, 3H).

Compound 28: 145 mg, 8% yield; LCMS: m/z=552.2 (M+H); rt 3.09 min; (LCMS method: Column: Column-Kinetex XB-C18 (75×3 mm-2.6 μm), Mobile phase A: 98% water: 2% acetonitrile; 10 mM ammonium formate; Mobile phase B: 2% Water: 98% acetonitrile; 10 mM ammonium formate; Flow: 1.0 mL/min; Temp: 50° C.; Time (min): 0-4; % B: 0-100%). ¹H NMR (400 MHz, DMSO-d₆) δ ppm 8.91 (s, 1H), 8.27-8.16 (m, 2H), 7.99 (d, J=9.0 Hz, 2H), 7.69-7.57 (m, 2H), 7.23-7.13 (m, 2H), 5.77-5.41 (m, 1H), 5.09-4.62 (m, 2H), 3.90-3.65 (m, 1H), 3.44 (s, 3H), 3.14-3.02 (m, 1H), 2.80-2.74 (m, 1H), 1.61-1.40 (m, 3H), 1.10-0.93 (m, 3H) [11H obscured with solvent peak].

### DGKi Compounds 29 and 30

### 4-((2S,5R)-4-(1-(4-(cyclopropylmethoxy)-2-fluorophenyl)propyl)-2,5-diethylpiperazin-1-yl)-1-methyl-2-oxo-1,2-dihydropyrido[3,2-d]pyrimidine-6-carbonitrile

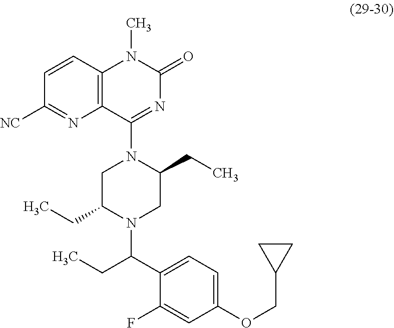 [SMILES: CCC(c1ccc(OCC2CC2)cc1F)N1C[C@H](CC)N(c2nc(=O)n(C)c3ccc(C#N)nc23)C[C@H]1CC]

To a stirred solution of 4-((2S,5R)-2,5-diethylpiperazin-1-yl)-1-methyl-2-oxo-1,2-dihydropyrido[3,2-d]pyrimidine-6-carbonitrile, HCl (200 mg, 0.55 mmol) in acetonitrile (5 mL) were added DIPEA (0.3 mL, 1.65 mmol), sodium iodide (83 mg, 0.55 mmol) and 1-(1-chloropropyl)-4-(cyclopropylmethoxy)-2-fluorobenzene (268 mg, 1.1 mmol). The reaction mixture was heated at 80° C. for 16 h. The reaction mixture was allowed to cool to room temperature. Another lot of 1-(1-chloropropyl)-4-(cyclopropylmethoxy)-2-fluorobenzene (268 mg, 1.102 mmol) was added and continued heating for another 16 h. The reaction mixture was cooled, the solvent was removed under reduced pressure and the residue was dissolved in ethyl acetate (10×20 mL). The organic layer was washed with brine, dried over Na₂SO₄, concentrated under reduced pressure to yield the crude product which was purified by preparative HPLC. HPLC method: Column: EXRS (20×250 mm, 5 μm), mobile phase A-10 mM ammonium acetate in water R, mobile phase A-B: acetonitrile, FLOW: 20 mL/min.

Fraction 1 was concentrated under reduced pressure and the product was diluted with (EtOH/H₂O, 1:5) and lyophilized to yield Compound 29 (35 mg, 11.6% yield); LCMS: m/z, 533.4 [M+H]+, rt 1.57 min; (LCMS method: Column: KINETIX XB C18 (75×3 mm, 2.6 μm); mobile phase A: 10 mM ammonium acetate in water (pH 3.3), mobile phase B: acetonitrile. ¹H NMR (DMSO-d₆, 400 MHz) δ (ppm) 8.23 (d, J=9.0 Hz, 1H), 7.97 (d, J=9.0 Hz, 1H), 7.33 (m, 1H), 6.62-6.92 (m, 2H), 5.29-6.06 (m, 1H), 4.70-5.05 (m, 1H), 3.82 (m, 3H), 3.43 (s, 3H), 2.99-3.10 (m, 1H), 2.80-2.87 (m, 1H), 2.63-2.78 (m, 1H), 2.33 (s, 1H), 1.74-2.11 (m, 3H), 1.51-1.66 (m, 1H), 1.17-1.46 (m, 3H), 0.84-1.01 (m, 3H), 0.61-0.78 (m, 6H), 0.53-0.61 (m, 2H), 0.29-0.35 (m, 2H).

Fraction 2 was concentrated under reduced pressure and the product was diluted with (EtOH/H₂O, 1:5) and lyophilized to yield Compound 30 (37 mg, 12.35% yield); LCMS: m/z, 533.4 [M+H]+, rt 2.72 min; [(LCMS Method: Column: KINETIX XB C18 (75×3 mm, 2.6 μm); mobile phase A: 10 mM ammonium acetate in water (pH 3.3), mobile phase B: acetonitrile. ¹H NMR (DMSO-d₆, 400 MHz): δ (ppm) 8.13-8.35 (m, 1H), 7.98 (m, 1H), 7.38 (m, 1H), 6.61-6.89 (m, 2H), 5.18-6.15 (m, 1H), 4.66-5.13 (m, 1H), 3.63-3.90 (m, 3H), 3.43 (s, 3H), 3.25 (m, 1H), 3.00-3.15 (m, 1H), 2.63-2.70 (m, 1H), 2.26-2.38 (m, 1H), 1.81 (m, 3H), 1.35-1.61 (m, 2H), 1.15-1.26 (m, 2H), 0.88-1.00 (m, 3H), 0.61-0.71 (m, 6H), 0.51-0.59 (m, 2H), 0.32 (m, 2H).

### DGKi Compounds 31 and 32

### 4-((2S,5R)-2,5-Diethyl-4-(1-(4-(trifluoromethyl)phenyl)butyl)piperazin-1-yl)-1-methyl-2-oxo-1,2-dihydropyrido[3,2-d]pyrimidine-6-carbonitrile

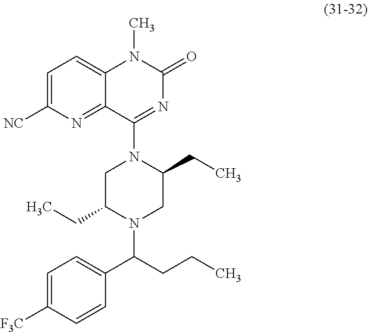 [SMILES: CCCC(c1ccc(C(F)(F)F)cc1)N1C[C@H](CC)N(c2nc(=O)n(C)c3ccc(C#N)nc23)C[C@H]1CC]

To a stirred solution of 4-((2S,5R)-2,5-diethylpiperazin-1-yl)-1-methyl-2-oxo-1,2-dihydropyrido[3,2-d]pyrimidine-6-carbonitrile, HCl (0.4 g, 1.1 mmol) in acetonitrile (10 mL) was added DIPEA (0.6 mL, 3.31 mmol), followed by 1-(1-chlorobutyl)-4-trifluoromethyl)benzene (0.783 g, 3.31 mmol) and sodium iodide (0.165 g, 1.102 mmol). The reaction mixture was heated at 85° C. for 16 h. The reaction mixture was filtered through a Celite pad, washed with ethyl acetate and the filtrate was concentrated under reduced pressure to give the crude compound, which was purified by preparative HPLC [HPLC Method: Column: YMC ExRS (250 mm×21.2 mm, 5 μm) Mobile phase A=10 mM ammoniuim acetate pH 4.5 in water. Mobile phase B=acetonitrile Gradient: 80% B over 2 minutes, then a 16 minute hold at 100% B; Flow: 19 mL/min) to yield Compounds 31 and 32.

Compound 31: (10 mg, 1.7% yield), LCMS: m/z=527.4 (M+H); rt 2.626 min; [LCMS Method: Column: XBridge BEH XP C18 (50×2.1 mm), 2.5 μm; Mobile phase A: 95% Water: 5% Acetonitrile; 10 mM NH₄OAC; Mobile phase B: 5% Water: 95% Acetonitrile; 10 mM NH₄OAC; Flow: 1.1 mL/min; Temp: 50° C.; Time (min)]. ¹H NMR (400 MHz, DMSO-d₆) δ 8.30-8.16 (m, 1H), 7.98 (d, J=9.0 Hz, 1H), 7.72 (d, J=8.3 Hz, 2H), 7.56 (br d, J=7.8 Hz, 2H), 5.86-5.44 (m, 1H), 5.01-4.77 (m, 1H), 3.730-3.718 (m, 1H), 3.46 (s, 3H), 3.43-3.35 (m, 1H) 3.13-3.01 (m, 1H), 2.93-2.75 (m, 2H), 2.38-2.26 (m, 1H), 2.17-1.74 (m, 3H), 1.63-1.22 (m, 3H), 1.01-0.86 (m, 4H), 0.84-0.75 (m, 3H), 0.73-0.54 (m, 3H).

Compound 32: (7.2 mg, 1.23% yield), LCMS: m/z=527.3 (M+H); rt 2.654 min; [LCMS Method: Column: XBridge BEH XP C18 (50×2.1) mm, 2.5 μm; Mobile phase A: 95% Water: 5% Acetonitrile; 10 mM NH₄OAC; Mobile phase B: 5% Water: 95% Acetonitrile; 10 mM NH4OAC; Flow: 1.1 mL/min; Temp: 50° C.; Time (min)]. ¹H NMR (400 MHz, DMSO-d₆) δ=8.29-8.15 (m, 1H), 7.96-8.02 (m, 1H), 7.70 (d, J=8.1 Hz, 2H), 7.58 (br d, J=8.1 Hz, 2H), 6.09-5.22 (m, 1H), 5.13-4.66 (m, 1H), 3.68-3.52 (m, 2H), 3.43 (s, 3H), 3.28-3.04 (m, 2H), 2.60-2.53 (m, 1H), 2.25-2.12 (m, 1H), 2.04-1.68 (m, 3H), 1.60-1.29 (m, 3H), 1.05-0.74 (m, 7H), 0.59 (t, J=7.5 Hz, 3H).

### DGKi Compounds 33 and 34

### 1-Methyl-4-((2S, 5R)-2-methyl-5-propyl-4-(1-(4-(trifluoromethyl)phenyl)ethyl)piperazin-1-yl)-2-oxo-1,2-dihydropyrido[3,2-d]pyrimidine-6-carbonitrile

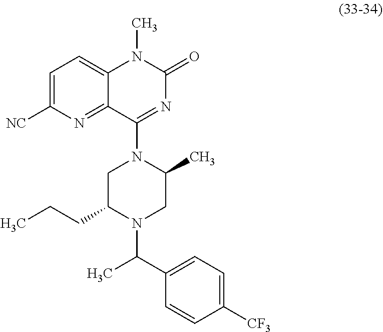 [SMILES: CCC[C@@H]1CN(c2nc(=O)n(C)c3ccc(C#N)nc23)[C@@H](C)CN1C(C)c1ccc(C(F)(F)F)cc1]

To a solution of 6-chloro-1-methyl-4-((2S,5R)-2-methyl-5-propyl-4-(1-(4-(trifluoromethyl)phenyl)ethyl)piperazin-1-yl)pyrido[3,2-d]pyrimidin-2(1H)-one (0.1 g, 0.19 mmol) in DMF (2 mL) were added zinc cyanide (0.046 g, 0.39 mmol), zinc (0.7 mg, 9.8 μmol) and triethylamine (0.1 mL, 0.59 mmol) followed by dichloro[9,9-dimethyl-4,5-bis(diphenylphosphino)xanthene]palladium(II) (0.015 g, 0.02 mmol) at room temperature under argon atmosphere. The reaction mixture was heated at 90° C. overnight. The reaction mixture was diluted with EtOAc (50 mL) and filtered through Celite® pad, washed with additional ethyl acetate (2×50 mL). The filtrate was washed with water (50 mL), brine, dried over Na₂SO₄ and concentrated under reduced pressure to yield the crude product, which was purified by preparative HPLC (HPLC method: Column: YMC EXRS (250×19 mm, 5 μm); mobile phase A: 10 mM ammonium acetate in water pH ˜4.5; mobile phase B: acetonitrile Flow: 20 mL/min) to yield Compound 33 and Compound 34.

COMPOUND 33: (13 mg, 14% yield). LCMS: m/z=499.3 [M+H]+; rt 2.376 min; (LCMS Method: Column: XBridge BEH XP C18 (50×2.1 mm, 2.5 μm); mobile phase A: 95% water: 5% acetonitrile; 10 mM NH₄OAc; mobile phase B: 5% water: 95% acetonitrile; 10 mM NH₄OAC; Flow: 1.1 mL/min; Temp: 50° C.). ¹H NMR (400 MHz, DMSO-d₆) δ (ppm)=8.22 (br d, J=8.8 Hz, 1H), 7.98 (d, J=8.8 Hz, 1H), 7.70-7.72 (m, 2H), 7.59-7.61 (m, 2H), 5.84-5.59 (m, 1H), 5.10-4.67 (m, 1H), 3.91-3.75 (m, 1H), 3.38-3.43 (m, 4H), 2.86-2.70 (m, 2H), 2.47-2.36 (m, 1H), 1.63-1.51 (m, 1H), 1.47-1.18 (m, 8H), 0.9-0.99 (m, 1H), 0.75-0.59 (m, 3H).

COMPOUND 34: (13 mg, 13% yield); LCMS: m/z=499.3 [M+H]+; rt 2.436 min; (LCMS Method: Column: XBridge BEH XP C18 (50×2.1 mm, 2.5 μm); mobile phase A: 95% water: 5% acetonitrile; 10 mM NH₄OAc; mobile phase B: 5% water: 95% acetonitrile; 10 mM NH₄OAC; Flow: 1.1 mL/min; Temp: 50° C.). ¹H NMR (400 MHz, DMSO-d₆) δ (ppm)=8.25 (br d, J=2.4 Hz, 1H), 8.06-7.92 (m, 1H), 7.77-7.65 (m, 2H), 7.65-7.54 (m, 2H), 6.09-5.44 (m, 1H), 5.04-4.68 (m, 1H), 3.81-3.59 (m, 2H), 3.44 (s, 3H), 3.28-3.13 (m, 1H), 2.52-2.61 (m, 1H), 2.24-2.05 (m, 1H), 1.72-1.48 (m, 2H), 1.47-1.15 (m, 8H), 0.98-0.75 (m, 3H).

Biological Assays

The pharmacological properties of the compounds described herein may be confirmed by a number of biological assays.

1. In Vitro DGK Inhibition Assays

The DGKα and DGK reactions were performed using either extruded liposome (DGKα and DGKζ LIPGLO assays) or detergent/lipid micelle substrate (DGKα and DGKζ assays). The reactions were carried out in 50 mM MOPS pH 7.5, 100 mM NaCl, 10 mM MgCl₂, 1 μM CaCl₂, and 1 mM DTT (assay buffer). The reactions using a detergent/lipid micelle substrate also contained 50 mM octyl B-D-glucopyranoside. The lipid substrate concentrations were 11 mM PS and 1 mM DAG for the detergent/lipid micelle reactions. The lipid substrate concentrations were 2 mM PS, 0.25 mM DAG, and 2.75 mM PC for the extruded liposome reactions. The reactions were carried out in 150 M ATP. The enzyme concentrations for the DGKα and DGKζ were 5 nM.

The compound inhibition studies were carried out as follows: 50 nL droplets of each test compound (top concentration 10 mM with 11 point, 3-fold dilution series for each compound) solubilized in DMSO were transferred to wells of a white 1536 well plate (Corning 3725). A 5 mL enzyme/substrate solution at 2× final reaction concentration was prepared by combining 2.5 mL 4× enzyme solution (20 nM DGKα or DGKζ (prepared as described below) in assay buffer) and 2.5 mL of either 4× liposome or 4× detergent/lipid micelle solution (compositions described below) and incubated at room temperature for 10 minutes. Next, 1 μL 2× enzyme/substrate solution was added to wells containing the test compound and reactions were initiated with the addition of 1 μL 300 uM ATP. The reactions were allowed to proceed for 1 hr, after which 2 μL Glo Reagent (Promega V9101) was added and incubated for 40 minutes. Next, 4 μL Kinase Detection Reagent was added and incubated for 30 minutes. Luminescence was recorded using an EnVision microplate reader. The percent inhibition was calculated from the ATP conversion generated by no enzyme control reactions for 100% inhibition and vehicle-only reactions for 0% inhibition. The compounds were evaluated at 11 concentrations to determine IC₅₀.

4× Detergent/Lipid Micelle Preparation

The detergent/lipid micelle was prepared by combining 15 g phosphatidylserine (Avanti 840035P) and 1 g diacylglycerol (8008110) and dissolving into 150 mL chloroform in a 2 L round bottom flask. Chloroform was removed under high vacuum by rotary evaporation. The resulting colorless, tacky oil was resuspended in 400 mL 50 mM MOPS pH 7.5, 100 mM NaCl, 20 mM NaF, 10 mM MgCl₂, 1 μM CaCl₂, 1 mM DTT, and 200 mM octyl glucoside by vigorous mixing. The lipid/detergent solution was split into 5 mL aliquots and stored at −80° C.

4× Liposome Preparation

The lipid composition was 5 mol % DAG (Avanti 8008110), 40 mol % PS (Avanti 840035P), and 55 mol % PC (Avanti 850457) at a total lipid concentration of 15.2 mg/mL for the 4× liposome solution. The PC, DAG, and PS were dissolved in chloroform, combined, and dried in vacuo to a thin film. The lipids were hydrated to 20 mM in 50 mM MOPS pH 7.5, 100 mM NaCl, 5 mM MgCl₂, and were freeze-thawed five times. The lipid suspension was extruded through a 100 nm polycarbonate filter eleven times. Dynamic light scattering was carried out to confirm liposome size (50-60 nm radius). The liposome preparation was stored at 4° C. for as long as four weeks.

Baculovirus Expression of Human DGKα and DGKζ

Human DGK-alpha-TVMV-His-pFBgate and human DGK-zeta-transcript variant-2-TVMV-His-pFBgate baculovirus samples were generated using the Bac-to-Bac baculovirus expression system (Invitrogen) according to the manufacturer's protocol. The DNA used for expression of DGK-alpha and DGK-zeta have SEQ ID NOs: 1 and 3, respectively. Baculovirus amplification was achieved using infected Sf9 cells at 1:1500 virus/cell ratios, and grown for 65 hours at 27° C. post-transfection.

The expression scale up for each protein was carried out in the Cellbag 50L WAVE-Bioreactor System 20/50 from GE Healthcare Bioscience. 12 L of 2×10⁶ cells/mL Sf9 cells (Expression System, Davis, Calif.) grown in ESF921 insect medium (Expression System) were infected with virus stock at 1:200 virus/cell ratios, and grown for 66-68 hours at 27° C. post-infection. The infected cell culture was harvested by centrifugation at 2000 rpm for 20 min 4° C. in a SORVALL® RC12BP centrifuge. The cell pellets were stored at −70° C. until purification.

Purification of Human DGK-Alpha and DGK-Zeta

Full length human DGKα and DGKζ, each expressed containing a TVMV-cleavable C-terminal Hexa-His tag sequence (SEQ ID NOs: 2 and 4, respectively) and produced as described above, were purified from Sf9 baculovirus-infected insect cell paste. The cells were lysed using nitrogen cavitation method with a nitrogen bomb (Parr Instruments), and the lysates were clarified by centrifugation. The clarified lysates were purified to ˜90% homogeneity, using three successive column chromatography steps on an ÄKTA Purifier Plus system. The three steps column chromatography included nickel affinity resin capture (i.e. HisTrap FF crude, GE Healthcare), followed by size exclusion chromatography (i.e. HiLoad 26/600 Superdex 200 prep grade, GE Healthcare for DGK-alpha, and HiPrep 26/600 Sephacryl S 300 HR, GE Healthcare for DGK-zeta). The third step was ion exchange chromatography, and differed for the two isoforms. DGKα was polished using Q-Sepharose anion exchange chromatography (GE Healthcare). DGKζ was polished using SP Sepharose cation exchange chromatography (GE Healthcare). The proteins were delivered at concentrations of >2 mg/mL. The formulation buffers were identical for both proteins: 50 mM Hepes, pH 7.2, 500 mM NaCl, 10% v/v glycerol, 1 mM TCEP, and 0.5 mM EDTA.

2. Raji CD4 T Cell IL2 Assay

A 1536-well IL-2 assay was performed in 4 μL volume using pre-activated CD4 T cells and Raji cells. Prior to the assay, CD4 T cells were pre-activated by treatment with α-CD3, α-CD28 and PHA at 1.5 μg/mL, 1 μg/mL, and 10 μg/mL, respectively. Raji cells were treated with Staphylococcal enterotoxin B (SEB) at 10,000 ng/mL. Serially diluted compounds were first transferred to 1536-well assay plate (Corning, #3727), followed by addition of 2 μL of pre-activated CD4 T cells (final density at 6000 cells/well) and 2 μL of SEB-treated Raji cells (2000 cells/well). After 24 hours incubation at a 37° C./5% CO₂ incubator, 4 μl of IL-2 detection reagents were added to the assay plate (Cisbio, #64IL2PEC). The assay plates were read on an Envision reader. To assess compound cytotoxicity, either Raji or CD4 T cells were incubated with the serially diluted compounds. After 24 hours incubation, 4 μL of Cell Titer Glo (Promega, #G7572) were added, and the plates were read on an Envision reader. The 50% effective concentration (IC₅₀) was calculated using the four-parameter logistic formula y=A+((B−A)/(1+((C/x)\hat{ }D))), where A and B denote minimal and maximal % activation or inhibition, respectively, C is the IC₅₀, D is hill slope and x represent compound concentration.

3. CellTiter-Glo CD8 T Cell Proliferation Assay

Frozen naïve human CD8 T cells were thawed in RPMI+10% FBS, incubated for 2 h in 37° C., and counted. The 384-well tissue culture plate was coated overnight at 4° C. with 20 μl anti-human CD3 at 0.1 μg/mL in plain RPMI, which was removed off the plate before 20k/40 μL CD8 T cells with 0.5 μg/ml soluble anti-human CD28 were added to each well. The compounds were echoed to the cell plate immediately after the cells were plated. After 72 h incubation at 37° C. incubator, 10 μL CellTiter-glo reagent (Promega catalog number G7570) was added to each well. The plate was vigorously shaken for 5 mins, incubated at room temperature for another 15 mins and read on Envision for CD8 T cell proliferation. In analysis, 0.1 μg/mL anti-CD3 and 0.5 μg/mL anti-CD28 stimulated CD8 T cell signal was background. The reference compound, 8-(4-(bis(4-fluorophenyl)methyl) piperazin-1-yl)-5-methyl-7-nitro-6-oxo-5,6-dihydro-1,5-naphthyridine-2-carbonitrile, at 3 μM was used to set the 100% range and EC₅₀ was at absolute 50% to normalize the data.

4. DGK APi-Reporter Assay

The Jurkat APi-luciferase Reporter was generated using the Cignal Lenti APi Reporter (luc) Kit from SABiosciences (CLS-011L).

The compounds were transferred from an Echo LDV plate to individual wells of a 384-well plate (white, solid-bottom, opaque PE CulturPlate 6007768) using an Echo550 instrument. The sample size was 30 nL per well; and one destination plate per source plate. The cell suspensions were prepared by transferring 40 mL cells (2×20 mL) to clean 50 mL conical tubes. The cells were concentrated by centrifugation (1200 rpm; 5 mins; ambient temperature). The supernatant was removed and all cells were suspended in RPMI (Gibco 11875)+10% FBS to make a 1.35×10⁶ cells/ml concentration. The cells were added manually using a multi-channel pipette, 30 L/well of cell suspension to a 384-well TC plate containing the compounds, 4.0×10⁴ cells per well. The cell plates were incubated for 20 minutes at 37° C. and 5% CO₂.

During the incubation, anti-CD3 antibody (αCD3) solutions were prepared by mixing 3 μL aCD3 (1.3 mg/mL) with 10 mL medium [final conc=0.4 μg/mL]. Next, 1.5 μl aCD3 (1.3 mg/mL) was mixed with 0.5 mL medium [final conc=4 μg/ml]. After 20 minutes, 10 μL medium was added to all wells in column 1, wells A to M, and 10 μL uCD3 (4 ug/mL) per well was added in column 1, rows N to P for reference. Then using a multi-channel pipette, 10 μL αCD3 (0.4 ug/mL) per well was added. The αCD3 stimulated +/−compound-treated cells were incubated at 37° C., 5% CO₂ for 6 hours.

During this incubation period, Steady-Glo (Promega E2520) reagent was slowly thawed to ambient temperature. Next, 20 μL Steady-Glo reagent per well was added using a multi-drop Combi-dispenser. Bubbles were removed by centrifugation (2000 rpm, ambient temperature, 10 secs). The cells were incubated at room temperature for 5 minutes. Samples were characterized by measuring the Relative Light Units (RLU) with an using Envision Plate Reader Instrument on a luminescence protocol. The data was analyzed using the reference compound, 8-(4-(bis(4-fluorophenyl)methyl)piperazin-1-yl)-5-methyl-7-nitro-6-oxo-5,6-dihydro-1,5-naphthyridine-2-carbonitrile, to normalize 100% inhibition.

5. Murine Cytotoxic T Lymphocyte Assay

An antigen-specific cytolytic T-cell (CTL) assay was developed to evaluate functionally the ability of DGKα and DGKζ inhibitors to enhance effector T cell mediated tumor cell killing activity. CD8+ T-cells isolated from the OT-1 transgenic mouse recognize antigen presenting cells, MC38, that present the ovalbumin derived peptide SIINFEKL. Recognition of the cognate antigen initiates the cytolytic activity of the OT-1 antigen-specific CD8+ T cells.

Functional CTL cells were generated as follows: OT-1 splenocytes from 8-12 week old mice were isolated and expanded in the presence of the SIINFEKL peptide at 1 g/mL and mIL2 at 10 U/mL. After three days, fresh media with mIL2 U/ml was added. On day 5 of the expansion, the CD8+ T cells were isolated and ready for use. Activated CTL cells may be stored frozen for 6 months. Separately, one million MC38 tumor cells were pulsed with 1 μg/mL of SIINFEKL-OVA peptide for 3 hours at 37° C. The cells were washed (3×) with fresh media to remove excess peptide. Finally, CTL cells that were pretreated with DGK inhibitors for 1 hour in a 96-well U bottom plate were combined with the antigen loaded MC38 tumor cells at a 1:10 ratio. The cells were then spun at 700 rpm for 5 min and placed in an incubator overnight at 37° C. After 24 hours, the supernatant was collected for analysis of IFN-γ cytokine levels by AlphaLisa purchased from Perkin Elmer.

6. PHA Proliferation Assay

Phytohaemagglutinin (PHA)-stimulated blast cells from frozen stocks were incubated in RPMI medium (Gibco, ThermoFisher Scientific, Waltham, Mass.) supplemented with 10% fetal bovine serum (Sigma Aldrich, St. Louis, Mo.) for one hour prior to adding to individual wells of a 384-well plate (10,000 cells per well). The compounds were transferred to individual wells of a 384-well plate and the treated cells are maintained at 37° C., 5% CO₂ for 72 h in culture medium containing human IL2 (20 ng/mL) prior to measuring growth using MTS reagent [3-(4,5-dimethyl-2-yl)-5-(3-carboxymethoxyphenyl)-2-(4-sulfophenyl)-2H-tetrazolium] following manufacturer's instructions (Promega, Madison, Wis.). Percent inhibition was calculated comparing values between IL2 stimulated (0% inhibition) and unstimulated control (100% inhibition). Inhibition concentration (IC₅₀) determinations were calculated based on 50% inhibition on the fold-induction between IL2 stimulated and unstimulated treatments.

7. Human CD8 T Cells IFN-γ Assay

Frozen naïve human CD8 T cells were thawed in AIM-V media, incubated for 2 h in 37° C., and counted. The 384-well tissue culture plate was coated overnight at 4° C. with 20 μL anti-human CD3 at 0.05 μg/mL in PBS, which was removed off the plate before 40,000 cells per 40 microliters CD8 T cells with 0.1 μg/mL soluble anti-human CD28 were added to each well. The compounds were transferred using an Echo liquid handler to the cell plate immediately after the cells were plated. After 20 h incubation at 37° C. incubator, 3 microliters per well supernatants transferred into a new 384-well white assay plate for cytokine measurement.

Interferon-γ (IFN-γ) was quantitated using the AlphLISA kit (Cat#AL217) as described by the manufacturer manual (Perkin Elmer). The counts from each well were converted to IFN-γ concentration (pg/mL). The compound EC₅₀ values were determined by setting 0.05 μg/mL anti-CD3 plus 0.1 μg/mL anti-CD28 as the baseline, and co-stimulation of 3 μM of the reference compound, 8-(4-(bis(4-fluorophenyl)methyl) piperazin-1-yl)-5-methyl-7-nitro-6-oxo-5,6-dihydro-1,5-naphthyridine-2-carbonitrile, with anti-CD3 plus anti-CD28 as 100% activation.

8. Human CD8 T Cells pERK Assay

Frozen naïve human CD8 T cells were thawed in AIM-V media, incubated for 2 h in 37° C., and counted. The CD8 positive T cells were added to 384-well tissue culture plate at 20,000 cells per well in AIM-V media. One compound was added to each well, then bead bound anti-human CD3 and anti-CD28 mAb were added at final concentration of 0.3 μg/mL. The cells were incubated at 37° C. for 10 minutes. The reaction was stopped by adding lysis buffer from the AlphaLISA Surefire kit. (Perkin Elmer, cat#ALSU-PERK-A). Lysate (5 μL per well) was transferred into a new 384-well white assay plate for pERK activation measurement.

Compound EC₅₀ was determined as setting anti-CD3 plus anti-CD28 as baseline, and co-stimulation of 3 μM 8-(4-(bis(4-fluorophenyl)methyl)piperazin-1-yl)-5-methyl-7-nitro-6-oxo-5,6-dihydro-1,5-naphthyridine-2-carbonitrile with anti-CD3 plus anti-CD28 as 100% activation.

9. Human Whole Blood IFN-γ Assay

Human venous whole blood (22.5 μL per well), obtained from healthy donors, was pre-treated with compounds for one hour at 37° C. in a humidified 95% air/5% CO₂ incubator. The blood was stimulated with 2.5 μL anti-human CD3 and anti-CD28 mAb at a final concentration of 1 μg/mL each for 24 hours at 37° C. IFN-γ in the supermatants was measured using AIphLNSA kit (Cat#AL217).

Compound EC₅₀ determined as setting anti-CD3 plus anti-CD28 as baseline, and co-stimulation of 3 μM of the reference compound, 8-(4-(bis(4-fluorophenyl)methyl) piperazin-1-yl)-5-methyl-7-nitro-6-oxo-5,6-dihydro-1,5-naphthyridine-2-carbonitrile, with anti-CD3 plus anti-CD28 as 100% activation.

TABLE A
In vitro DGK Inhibition IC₅₀ Activity Values

| | | | | | INFg |
|---|---|---|---|---|---|
| | | | | | Whole |
| | | | HuCD8 | | Blood |
| Ex. | | | INFG | | Normal- |
| DGKi | DGKa | DGKz | Normal- | msCTL | ized |
| Compound | LIPGLO | LIPGLO | ized | INFg | Agonist |
| No. | IC₅₀ (μM) | IC₅₀ (μM) | EC₅₀ (μM) | IC₅₀ (μM) | EC₅₀ (μM) |
| 1 | 23 | 42 | — | — | — |
| 2 | 240 | 1.7 | 0.062 | 0.064 | 0.52 |
| 4 | 0.98 | 0.85 | 0.017 | 0.043 | 0.40 |
| 5 | 0.87 | 1.6 | 0.030 | — | 0.68 |
| 6 | 0.66 | 0.42 | 0.039 | 0.033 | 0.70 |
| 7 | 0.46 | 0.51 | 0.047 | 0.064 | 0.57 |
| 8 | 15 | 20 | 12 | — | — |
| 9 | 4.4 | 0.97 | 0.17 | — | 1.5 |
| 10 | 0.67 | 0.32 | 0.023 | 0.032 | 0.44 |
| 11 | 0.14 | 0.57 | 0.022 | — | 0.65 |
| 12 | 0.57 | 0.46 | 0.048 | — | 0.44 |
| 13 | 0.15 | 0.33 | 0.020 | — | 0.13 |
| 14 | 1.7 | 3.7 | — | — | — |
| 15 | 3.4 | 0.21 | — | — | — |
| 16 | 1.5 | 0.87 | 0.0095 | 0.072 | 0.52 |
| 17 | 0.16 | 0.13 | — | — | 0.02 |
| 18 | 4.66 | 2.44 | — | — | 3.75 |
| 19 | 0.39 | 0.81 | — | — | 0.52 |
| 20 | 0.12 | 0.044 | — | — | 0.032 |
| 21 | 0.094 | 0.060 | — | — | 0.043 |
| 22 | 0.099 | 0.054 | — | — | 0.020 |
| 23 | 0.15 | 0.29 | — | — | 10.48 |
| 24 | 0.32 | 0.13 | — | — | 0.20 |
| 25 | 0.067 | 0.029 | — | — | 0.015 |
| 26 | 0.014 | 0.033 | — | — | 0.090 |
| 27 | 0.19 | 0.44 | — | — | — |
| 28 | 0.18 | 0.56 | — | — | 0.24 |
| 29 | — | 0.038 | — | — | — |
| 30 | — | 0.92 | — | — | 11.3 |
| 31 | — | 0.039 | — | — | — |
| 33 | — | 0.064 | — | — | 20 |
| 34 | — | 1.30 | — | — | — |

The compounds described herein possess activity as an inhibitor(s) of one or both of the DGKα and DGKζ enzymes, and therefore, may be used in the treatment of diseases associated with the inhibition of DGKα and DGKζ activity.

Nucleotide sequence encoding hDGKu-(M1-S735)-Ct-TVMV-His:

(SEQ ID NO: 1)
0001 ATGGCCAAGG AGAGGGGCCT AATAAGCCCC AGTGATTTTG CCCAGCTGCA
0051 AAAATACATG GAATACTCCA CCAAAAAGGT CAGTGATGTC CTAAAGCTCT
0101 TCGAGGATGG CGAGATGGCT AAATATGTCC AAGGAGATGC CATTGGGTAC
0151 GAGGGATTCC AGCAATTCCT GAAAATCTAT CTCGAAGTGG ATAATGTTCC
0201 CAGACACCTA AGCCTGGCAC TGTTTCAATC CTTTGAGACT GGTCACTGCT
0251 TAAATGAGAC AAATGTGACA AAAGATGTGG TGTGTCTCAA TGATGTTTCC
0301 TGCTACTTTT CCCTTCTGGA GGGTGGTCGG CCAGAAGACA AGTTAGAATT
0351 CACCTTCAAG CTGTACGACA CGGACAGAAA TGGGATCCTG GACAGCTCAG
0401 AAGTGGACAA AATTATCCTA CAGATGATGC GAGTGGCTGA ATACCTGGAT
0451 TGGGATGTGT CTGAGCTGAG GCCGATTCTT CAGGAGATGA TGAAAGAGAT
0501 TGACTATGAT GGCAGTGGCT CTGTCTCTCA AGCTGAGTGG GTCCGGGCTG
0551 GGGCCACCAC CGTGCCACTG CTAGTGCTGC TGGGTCTGGA GATGACTCTG
0601 AAGGACGACG GACAGCACAT GTGGAGGCCC AAGAGGTTCC CCAGACCAGT
0651 CTACTGCAAT CTGTGCGAGT CAAGCATTGG TCTTGGCAAA CAGGGACTGA
0701 GCTGTAACCT CTGTAAGTAC ACTGTTCACG ACCAGTGTGC CATGAAAGCC
0751 CTGCCTTGTG AAGTCAGCAC CTATGCCAAG TCTCGGAAGG ACATTGGTGT
0801 CCAATCACAT GTGTGGGTGC GAGGAGGCTG TGAGTCCGGG CGCTGCGACC
0851 GCTGTCAGAA AAAGATCCGG ATCTACCACA GTCTGACCGG GCTGCATTGT
0901 GTATGGTGCC ACCTAGAGAT CCACGATGAC TGCCTGCAAG CGGTGGGCCA
0951 TGAGTGTGAC TGTGGGCTGC TCCGGGATCA CATCCTGCCT CCATCTTCCA
1001 TCTATCCCAG TGTCCTGGCC TCTGGACCGG ATCGTAAAAA TAGCAAAACA
1051 AGCCAGAAGA CCATGGATGA TTTAAATTTG AGCACCTCTG AGGCTCTGCG
1101 GATTGACCCT GTTCCTAACA CCCACCCACT TCTCGTCTTT GTCAATCCTA
1151 AGAGTGGCGG GAAGCAGGGG CAGAGGGTGC TCTGGAAGTT CCAGTATATA
1201 TTAAACCCTC GACAGGTGTT CAACCTCCTA AAGGATGGTC CTGAGATAGG
1251 GCTCCGATTA TTCAAGGATG TTCCTGATAG CCGGATTTTG GTGTGTGGTG
1301 GAGACGGCAC AGTAGGCTGG ATTCTAGAGA CCATTGACAA AGCTAACTTG
1351 CCAGTTTTGC CTCCTGTTGC TGTGTTGCCC CTGGGTACTG GAAATGATCT
1401 GGCTCGATGC CTAAGATGGG GAGGAGGTTA TGAAGGACAG AATCTGGCAA
1451 AGATCCTCAA GGATTTAGAG ATGAGTAAAG TGGTACATAT GGATCGATGG
1501 TCTGTGGAGG TGATACCTCA ACAAACTGAA GAAAAAAGTG ACCCAGTCCC
1551 CTTTCAAATC ATCAATAACT ACTTCTCTAT TGGCGTGGAT GCCTCTATTG
1601 CTCATCGATT CCACATCATG CGAGAGAAAT ATCCGGAGAA GTTCAACAGC
1651 AGAATGAAGA ACAAGCTATG GTACTTCGAA TTTGCCACAT CTGAATCCAT
1701 CTTCTCAACA TGCAAAAAGC TGGAGGAGTC TTTGACAGTT GAGATCTGTG
1751 GGAAACCGCT GGATCTGAGC AACCTGTCCC TAGAAGGCAT CGCAGTGCTA
1801 AACATCCCTA GCATGCATGG TGGCTCCAAC CTCTGGGGTG ATACCAGGAG
1851 ACCCCATGGG GATATCTATG GGATCAACCA GGCCTTAGGT GCTACAGCTA
1901 AAGTCATCAC CGACCCTGAT ATCCTGAAAA CCTGTGTACC AGACCTAAGT
1951 GACAAGAGAC TGGAAGTGGT TGGGCTGGAG GGTGCAATTG AGATGGGCCA
2001 AATCTATACC AAGCTCAAGA ATGCTGGACG TCGGCTGGCC AAGTGCTCTG
2051 AGATCACCTT CCACACCACA AAAACCCTTC CCATGCAAAT TGACGGAGAA
2101 CCCTGGATGC AGACGCCCTG TACAATCAAG ATCACCCACA AGAACCAGAT
2151 GCCCATGCTC ATGGGCCCAC CCCCCCGCTC CACCAATTTC TTTGGCTTCT
2201 TGAGCGGATC CTCGGAGACA GTGCGGTTTC AGGGACACCA CCACCATCAC
2251 CACTGA

Amino acid sequence of hDGKu-(M1-S735)-Ct-TVMV-His:

(SEQ ID NO: 2)
0001 MAKERGLISP SDFAQLQKYM EYSTKKVSDV LKLFEDGEMA KYVQGDAIGY EGFQQFLKIY 0060
0061 LEVDNVPRHL SLALFQSFET GHCLNETNVT KDVVCLNDVS CYFSLLEGGR PEDKLEFTFK 0120
0121 LYDTDRNGIL DSSEVDKIIL QMMRVAEYLD WDVSELRPIL QEMMKEIDYD GSGSVSQAEW 0180
0181 VRAGATTVPL LVLLGLEMTL KDDGQHMWRP KRFPRPVYCN LCESSIGLGK QGLSCNLCKY 0240
0241 TVHDQCAMKA LPCEVSTYAK SRKDIGVQSH VWVRGGCESG RCDRCQKKIR IYHSLTGLHC 0300
0301 VWCHLEIHDD CLQAVGHECD CGLLRDHILP PSSIYPSVLA SGPDRKNSKT SQKTMDDLNL 0360
0361 STSEALRIDP VPNTHPLLVF VNPKSGGKQG QRVLWKFQYI LNPRQVFNLL KDGPEIGLRL 0420
0421 FKDVPDSRIL VCGGDGTVGW ILETIDKANL PVLPPVAVLP LGTGNDLARC LRWGGGYEGQ 0480
0481 NLAKILKDLE MSKWHMDRVV SVEVIPQQTE EKSDPVPFQI INNYFSIGVD ASIAHRFHIM 0540
0541 REKYPEKFNS RMKNKLWYFE FATSESIFST CKKLEESLTV EICGKPLDLS NLSLEGIAVL 0600
0601 NIPSMHGGSN LWGDTRRPHG DIYGINQALG ATAKVITDPD ILKTCVPDLS DKRLEVVGLE 0660
0661 GAIEMGQIYT KLKNAGRRLA KCSEITFHTT KTLPMQIDGE PWMQTPCTIK ITHKNQMPML 0720
0721 MGPPPRSTNF FGFLSGSSET VRFQGHHHHH H 0751

Nucleotide sequence encoding hDGK ζ-(M1-A928)-transcript variant-2 Ct-TVMV-His:

(SEQ ID NO: 3)
0001 ATGGAGCCGC GGGACGGTAG CCCCGAGGCC CGGAGCAGCG ACTCCGAGTC
0051 GGCTTCCGCC TCGTCCAGCG GCTCCGAGCG CGACGCCGGT CCCGAGCCGG
0101 ACAAGGCGCC GCGGCGACTC AACAAGCGGC GCTTCCCGGG GCTGCGGCTC
0151 TTCGGGCACA GGAAAGCCAT CACGAAGTCG GGCCTCCAGC ACCTGGCCCC
0201 CCCTCCGCCC ACCCCTGGGG CCCCGTGCAG CGAGTCAGAG CGGCAGATCC
0251 GGAGTACAGT GGACTGGAGC GAGTCAGCGA CATATGGGGA GCACATCTGG
0301 TTCGAGACCA ACGTGTCCGG GGACTTCTGC TACGTTGGGG AGCAGTACTG
0351 TGTAGCCAGG ATGCTGCAGA AGTCAGTGTC TCGAAGAAAG TGCGCAGCCT
0401 GCAAGATTGT GGTGCACACG CCCTGCATCG AGCAGCTGGA GAAGATAAAT
0451 TTCCGCTGTA AGCCGTCCTT CCGTGAATCA GGCTCCAGGA ATGTCCGCGA
0501 GCCAACCTTT GTACGGCACC ACTGGGTACA CAGACGACGC CAGGACGGCA
0551 AGTGTCGGCA CTGTGGGAAG GGATTCCAGC AGAAGTTCAC CTTCCACAGC
0601 AAGGAGATTG TGGCCATCAG CTGCTCGTGG TGCAAGCAGG CATACCACAG
0651 CAAGGTGTCC TGCTTCATGC TGCAGCAGAT CGAGGAGCCG TGCTCGCTGG
0701 GGGTCCACGC AGCCGTGGTC ATCCCGCCCA CCTGGATCCT CCGCGCCCGG
0751 AGGCCCCAGA ATACTCTGAA AGCAAGCAAG AAGAAGAAGA GGGCATCCTT
0801 CAAGAGGAAG TCCAGCAAGA AAGGGCCTGA GGAGGGCCGC TGGAGACCCT
0851 TCATCATCAG GCCCACCCCC TCCCCGCTCA TGAAGCCCCT GCTGGTGTTT
0901 GTGAACCCCA AGAGTGGGGG CAACCAGGGT GCAAAGATCA TCCAGTCTTT
0951 CCTCTGGTAT CTCAATCCCC GACAAGTCTT CGACCTGAGC CAGGGAGGGC
1001 CCAAGGAGGC GCTGGAGATG TACCGCAAAG TGCACAACCT GCGGATCCTG
1051 GCGTGCGGGG GCGACGGCAC GGTGGGCTGG ATCCTCTCCA CCCTGGACCA
1101 GCTACGCCTG AAGCCGCCAC CCCCTGTTGC CATCCTGCCC CTGGGTACTG
1151 GCAACGACTT GGCCCGAACC CTCAACTGGG GTGGGGGCTA CACAGATGAG
1201 CCTGTGTCCA AGATCCTCTC CCACGTGGAG GAGGGGAACG TGGTACAGCT
1251 GGACCGCTGG GACCTCCACG CTGAGCCCAA CCCCGAGGCA GGGCCTGAGG
1301 ACCGAGATGA AGGCGCCACC GACCGGTTGC CCCTGGATGT CTTCAACAAC
1351 TACTTCAGCC TGGGCTTTGA CGCCCACGTC ACCCTGGAGT TCCACGAGTC
1401 TCGAGAGGCC AACCCAGAGA AATTCAACAG CCGCTTTCGG AATAAGATGT
1451 TCTACGCCGG GACAGCTTTC TCTGACTTCC TGATGGGCAG CTCCAAGGAC
1501 CTGGCCAAGC ACATCCGAGT GGTGTGTGAT GGAATGGACT TGACTCCCAA
1551 GATCCAGGAC CTGAAACCCC AGTGTGTTGT TTTCCTGAAC ATCCCCAGGT
1601 ACTGTGCGGG CACCATGCCC TGGGGCCACC CTGGGGAGCA CCACGACTTT
1651 GAGCCCCAGC GGCATGACGA CGGCTACCTC GAGGTCATTG GCTTCACCAT
1701 GACGTCGTTG GCCGCGCTGC AGGTGGGCGG ACACGGCGAG CGGCTGACGC
1751 AGTGTCGCGA GGTGGTGCTC ACCACATCCA AGGCCATCCC GGTGCAGGTG
1801 GATGGCGAGC CCTGCAAGCT TGCAGCCTCA CGCATCCGCA TCGCCCTGCG
1851 CAACCAGGCC ACCATGGTGC AGAAGGCCAA GCGGCGGAGC GCCGCCCCCC
1901 TGCACAGCGA CCAGCAGCCG GTGCCAGAGC AGTTGCGCAT CCAGGTGAGT
1951 CGCGTCAGCA TGCACGACTA TGAGGCCCTG CACTACGACA AGGAGCAGCT
2001 CAAGGAGGCC TCTGTGCCGC TGGGCACTGT GGTGGTCCCA GGAGACAGTG
2051 ACCTAGAGCT CTGCCGTGCC CACATTGAGA GACTCCAGCA GGAGCCCGAT
2101 GGTGCTGGAG CCAAGTCCCC GACATGCCAG AAACTGTCCC CCAAGTGGTG
2151 CTTCCTGGAC GCCACCACTG CCAGCCGCTT CTACAGGATC GACCGAGCCC
2201 AGGAGCACCT CAACTATGTG ACTGAGATCG CACAGGATGA GATTTATATC
2251 CTGGACCCTG AGCTGCTGGG GGCATCGGCC CGGCCTGACC TCCCAACCCC
2301 CACTTCCCCT CTCCCCACCT CACCCTGCTC ACCCACGCCC CGGTCACTGC
2351 AAGGGGATGC TGCACCCCCT CAAGGTGAAG AGCTGATTGA GGCTGCCAAG
2401 AGGAACGACT TCTGTAAGCT CCAGGAGCTG CACCGAGCTG GGGGCGACCT
2451 CATGCACCGA GACGAGCAGA GTCGCACGCT CCTGCACCAC GCAGTCAGCA
2501 CTGGCAGCAA GGATGTGGTC CGCTACCTGC TGGACCACGC CCCCCCAGAG
2551 ATCCTTGATG CGGTGGAGGA AAACGGGGAG ACCTGTTTGC ACCAAGCAGC
2601 GGCCCTGGGC CAGCGCACCA TCTGCCACTA CATCGTGGAG GCCGGGGCCT
2651 CGCTCATGAA GACAGACCAG CAGGGCGACA CTCCCCGGCA GCGGGCTGAG
2701 AAGGCTCAGG ACACCGAGCT GGCCGCCTAC CTGGAGAACC GGCAGCACTA
2751 CCAGATGATC CAGCGGGAGG ACCAGGAGAC GGCTGTGGGA TCCTCGGAGA
2801 CAGTGCGGTT TCAGGGACAC CACCACCATC ACCACTGA

Amino acid sequence of hDGKζ-(M1-A928)-transcript variant-2 Ct-TVMV-His:

(SEQ ID NO: 4)
0001 MEPRDGSPEA RSSDSESASA SSSGSERDAG PEPDKAPRRL NKRRFPGLRL FGHRKAITKS 0060
0061 GLQHLAPPPP TPGAPCSESE RQIRSTVDWS ESATYGEHIW FETNVSGDFC YVGEQYCVAR 0120
0121 mLQKSVSRRK CAACKIVVHT PCIEQLEKIN FRCKPSFRES GSRNVREPTF VRHHWVHRRR 0180
0181 QDGKCRHCGK GFQQKFTFHS KEIVAISCSW CKQAYHSKVS CFMLQQIEEP CSLGVHAAVV 0240
0241 IPPTWILRAR RPQNTLKASK KKKRASFKRK SSKKGPEEGR WRPFIIRPTP SPLMKPLLVF 0300
0301 VNPKSGGNQG AKIIQSFLWY LNPRQVFDLS QGGPKEALEM YRKVHNLRIL ACGGDGTVGW 0360
0361 ILSTLDQLRL KPPPPVAILP LGTGNDLART LNWGGGYTDE PVSKILSHVE EGNVVQLDRW 0420
0421 DLHAEPNPEA GPEDRDEGAT DRLPLDVFNN YFSLGFDAHV TLEFHESREA NPEKFNSRFR 0480
0481 NKMFYAGTAF SDFLMGSSKD LAKHIRVVCD GMDLTPKIQD LKPQCVVFLN IPRYCAGTMP 0540
0541 WGHPGEHHDF EPQRHDDGYL EVIGFTMTSL AALQVGGHGE RLTQCREVVL TTSKAIPVQV 0600
0601 DGEPCKLAAS RIRIALRNQA TMVQKAKRRS AAPLHSDQQP VPEQLRIQVS RVSMHDYEAL 0660
0661 HYDKEQLKEA SVPLGTVVVP GDSDLELCRA HIERLQQEPD GAGAKSPTCQ KLSPKWCFLD 0720
0721 ATTASRFYRI DRAQEHLNYV TEIAQDEIYI LDPELLGASA RPDLPTPTSP LPTSPCSPTP 0780
0781 RSLQGDAAPP QGEELIEAAK RNDFCKLQEL HRAGGDLMHR DEQSRTLLHH AVSTGSKDVV 0840
0841 RYLLDHAPPE ILDAVEENGE TCLHQAAALG QRTICHYIVE AGASLMKTDQ QGDTPRQRAE 0900
0901 KAQDTELAAY LENRQHYQMI QREDQETAVG SSETVRFQGH HHHHH 0945

1. A method of treating cancer in a subject, comprising administering to the subject a therapeutically effective amount of an inhibitor of DGKα and/or DGKζ and an antagonist of the PD1/PD-L1 axis.

2. A method of treating cancer in a subject, comprising administering to the subject a therapeutically effective amount of an inhibitor of DGKα and/or DGKζ and an antagonist of CTLA4.

3. A method of treating cancer in a subject, comprising administering to the subject a therapeutically effective amount of an inhibitor of DGKα and/or DGKζ, an antagonist of the PD1/PD-L1 axis and an antagonist of CTLA4.

4. The method of any one of claims 1-3, wherein the inhibitor of human DGKα and/or DGKζ is an inhibitor of DGKα and not a significant inhibitor of DGKζ.

5. The method of any one of claims 1-3, wherein the inhibitor of DGKα and/or DGKζ is an inhibitor of DGKζ and not a significant inhibitor of DGKα.

6. The method of any one of claims 1-5, wherein the inhibitor of DGKα and/or DGKζ is an inhibitor of DGKα and DGKζ.

7. The method of any one of claims 1-6, wherein the inhibitor of DGKα and/or DGKζ is not a significant inhibitor of other DGKs.

8. The method of any one of claims 1 and 3-7, wherein the antagonist of the PD1/PD-L1 axis is an antagonist of PD1, e.g., human PD1.

9. The method of claim 8, wherein the antagonist of PD-1 is nivolumab, pembrolizumab, or any other PD-1 antagonist described herein.

10. The method of any one of claims 1 and 3-7, wherein the antagonist of the PD1/PD-L1 axis is an antagonist of PD-L1, such as human PD-L1.

11. The method of claim 10, wherein the antagonist of PD-L1 is atezolizumab or any other PD-L1 antagonist described herein.

12. The method of any one of claims 2-11, wherein the antagonist of CTLA4 is ipilimumab or any other CTLA4 antagonist described herein.

13. The method of any one of claims 1-12, wherein the DGKα and/or DGKζ antagonist increases primary T cell signaling, as evidenced, e.g., by an increase in pERK/pPKC signaling.

14. The method of any one of claims 1-13, wherein the inhibitor of DGKα and/or DGKζ lowers the threshold for antigen stimulation; lowers the affinity requirement and/or lowers the concentration requirement of antigen for T cell antigen recognition and activation.

15. The method of any one of claims 1-14, wherein the inhibitor of DGKα and/or DGKζ increases CTL effector function.

16. The method of any one of claims 1-15, wherein the inhibitor of DGKα and/or DGKζ enhances tumor cell killing.

17. The method of any one of claims 1-16, wherein the anti-tumor activity of the inhibitor of DGKα and/or DGKζ is dependent on CD8+ T cells in the CT26 animal model.

18. The method of any one of claims 1-17, wherein the anti-tumor activity of the inhibitor of DGKα and/or DGKζ is dependent on NK cells in the CT26 animal model.

19. The method of any one of claims 1-18, wherein the anti-tumor activity of the inhibitor of DGKα and/or DGKζ is enhanced by CD4 cell depletion in the CT-26 animal model.

20. The method of any one of claims 1-19, wherein the inhibitor of DGKα and/or DGKζ enhances AH1+ Tetramer antigen presentation in the CT-26 animal model or overcomes decreased B2M levels to restore T cell effector function.

21. The method of any one of claims 1-20, wherein the inhibitor of DGKα and/or DGKζ is a compound of Formula (I): 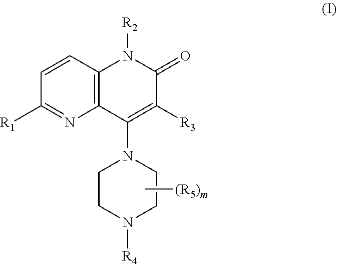
or a pharmaceutically acceptable salt thereof, wherein:
R₁ is H, F, Cl, Br, —CN, C₁₋₃ alkyl substituted with zero to 4 R₁ₐ, C₃₋₄ cycloalkyl substituted with zero to 4 R₁ₐ, C₁₋₃ alkoxy substituted with zero to 4 R₁ₐ, —NRₐRₐ, —S(O)ₙRₑ, or —P(O)RₑRₑ;
each R₁ₐ is independently F, Cl, —CN, —OH, —OCH₃, or —NRₐRₐ;
each Rₐ is independently H or C₁₋₃ alkyl;
each Rₑ is independently C₃₋₄ cycloalkyl or C₁₋₃ alkyl substituted with zero to 4 R₁ₐ;
R₂ is H, C₁₋₃ alkyl substituted with zero to 4 R₂ₐ, or C₃₋₄ cycloalkyl substituted with zero to 4 R₂ₐ;
each R₂ₐ is independently F, Cl, —CN, —OH, —O(C₁₋₂ alkyl), C₃₋₄ cycloalkyl, C₃₋₄ alkenyl, or C₃₋₄ alkynyl;
R₃ is H, F, Cl, Br, —CN, C₁₋₃ alkyl, C₁₋₂ fluoroalkyl, C₃₋₄ cycloalkyl, C₃₋₄ fluorocycloalkyl, or —NO₂;
R₄ is —CH₂R₄ₐ, —CH₂CH₂R₄ₐ, —CH₂CHR₄ₐR₄ₐ, —CHR₄ₐR_{4b}, or —CR₄ₐR_{4b}R_{4c};
R₄ₐ and R_{4b} are independently:
(i) C₁₋₆ alkyl substituted with zero to 4 substituents independently selected from F, Cl, —CN, —OH, —OCH₃, —SCH₃, C₁₋₃ fluoroalkoxy, —NRₐRₐ, —S(O)₂Rₑ, or —NRₐS(O)₂Rₑ;
(ii) C₃₋₆ cycloalkyl, heterocyclyl, phenyl, or heteroaryl, each substituted with zero to 4 substituents independently selected from F, Cl, Br, —CN, —OH, C₁₋₆ alkyl, C₁₋₃ fluoroalkyl, C₁₋₄ hydroxyalkyl, —(CH₂)₁₋₂O(C₁₋₃ alkyl), C₁₋₄ alkoxy, —O(C₁₋₄ hydroxyalkyl), —O(CH)₁₋₃O(C₁₋₃ alkyl), C₁₋₃ fluoroalkoxy, —O(CH)₁₋₃NR_{c}R_{c}, —OCH₂CH═CH₂, —OCH₂C—CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₃ alkyl)₂, —S(O)₂(C₁₋₃ alkyl), —O(CH₂)₁₋₂(C₃₋₆ cycloalkyl), —O(CH₂)₁₋₂(morpholinyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, and R_{d}; or
(iii) C₁₋₄ alkyl substituted with one cyclic group selected from C₃₋₆ cycloalkyl, heterocyclyl, aryl, and heteroaryl, said cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C═CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₆ cycloalkyl;
or R₄ₐ and R_{4b} together with the carbon atom to which they are attached form a C₃₋₆ cycloalkyl or a 3- to 6-membered heterocyclyl, each substituted with zero to 3 R_{f};
each R_{f} is independently F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C═CH, —NR_{c}R_{c}, or a cyclic group selected from C₃₋₆ cycloalkyl, 3- to 6-membered heterocyclyl, phenyl, monocyclic heteroaryl, and bicyclic heteroaryl, each cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, and —NR_{c}R_{c};
R_{4c} is C₁₋₆ alkyl or C₃₋₆ cycloalkyl, each substituted with zero to 4 substituents independently selected from F, Cl, —OH, C₁₋₂ alkoxy, C₁₋₂ fluoroalkoxy, and —CN;
R_{4d} is —OCH₃;
each R_{c} is independently H or C₁₋₂ alkyl;
R_{d} is phenyl substituted with zero to 1 substituent selected from F, Cl, —CN, —CH₃, and —OCH₃;
each R₅ is independently —CN, C₁₋₆ alkyl substituted with zero to 4 R_{g}, C₂₋₄ alkenyl substituted with zero to 4 R_{g}, C₂₋₄ alkynyl substituted with zero to 4 R_{g}, C₃₋₄ cycloalkyl substituted with zero to 4 R_{g}, phenyl substituted with zero to 4 R_{g}, oxadiazolyl substituted with zero to 3 R_{g}, pyridinyl substituted with zero to 4 R_{g}, —(CH₂)₁₋₂ (heterocyclyl substituted with zero to 4 R_{g}), —(CH₂)₁₋₂NR_{c}C(O)(C₁₋₄ alkyl), —(CH₂)₁₋₂NR_{c}C(O)O(C₁₋₄ alkyl), —(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl);
each R_{g} is independently F, Cl, —CN, —OH, C₁₋₃ alkoxy, C₁₋₃ fluoroalkoxy, —O(CH₂)₁₋₂O(C₁₋₂ alkyl), or —NR_{c}R_{c};
m is zero, 1, 2, or 3; and
n is zero, 1, or 2.

22. The method of claim 21, wherein the inhibitor of DGKα and/or DGKζ is a compound of Formula (I) or a pharmaceutically acceptable salt thereof, wherein:
R₁ is H, F, Cl, Br, —CN, C₁₋₃ alkyl substituted with zero to 4 R₁ₐ, cyclopropyl substituted with zero to 3 R₁ₐ, C₁₋₃ alkoxy substituted with zero to 3 R₁ₐ, —NRₐRₐ, —S(O)ₙCH₃, or —P(O)(CH₃)₂;
each R₁ₐ is independently F, Cl, or —CN;
each Rₐ is independently H or C₁₋₃ alkyl;
R₂ is H or C₁₋₂ alkyl substituted with zero to 2 R₂ₐ;
each R₂ₐ is independently F, Cl, —CN, —OH, —O(C₁₋₂ alkyl), cyclopropyl, C₃₋₄ alkenyl, or C₃₋₄ alkynyl;
R₃ is H, F, Cl, Br, —CN, C₁₋₂ alkyl, —CF₃, cyclopropyl, or —NO₂;
R₄ₐ and R_{4b} are independently:
(i) C₁₋₄ alkyl substituted with zero to 4 substituents independently selected from F, Cl, —CN, —OH, —OCH₃, —SCH₃, C₁₋₃ fluoroalkoxy, and —NRₐRₐ;
(ii) C₃₋₆ cycloalkyl, heterocyclyl, phenyl, or heteroaryl, each substituted with zero to 4 substituents independently selected from F, Cl, Br, —CN, —OH, C₁₋₆ alkyl, C₁₋₃ fluoroalkyl, —CH₂OH, —(CH₂)₁₋₂O(C₁₋₂ alkyl), C₁₋₄ alkoxy, —O(C₁₋₄ hydroxyalkyl), —O(CH)₁₋₂O(C₁₋₂ alkyl), C₁₋₃ fluoroalkoxy, —O(CH)₁₋₂NR_{c}R_{c}, —OCH₂CH═CH₂, —OCH₂C—CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₂ alkyl)₂, —S(O)₂(C₁₋₃ alkyl), —O(CH₂)₁₋₂(C₃₋₄ cycloalkyl), —O(CH₂)₁₋₂(morpholinyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, and R_{d}; or
(iii) C₁₋₃ alkyl substituted with one cyclic group selected from C₃₋₆ cycloalkyl, heterocyclyl, phenyl, and heteroaryl, said cyclic group substituted with zero to 3 substituents independently selected from F, Cl, Br, —OH, —CN, C₁₋₃ alkyl, C₁₋₂ fluoroalkyl, C₁₋₃ alkoxy, C₁₋₂ fluoroalkoxy, —OCH₂CH═CH₂, —OCH₂C═CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₄ cycloalkyl;
or R₄ₐ and R_{4b} together with the carbon atom to which they are attached, form a C₃₋₆ cycloalkyl or a 3- to 6-membered heterocyclyl, each substituted with zero to 3 R_{f};
each R_{f} is independently F, Cl, Br, —OH, —CN, C₁₋₄ alkyl, C₁₋₂ fluoroalkyl, C₁₋₃ alkoxy, C₁₋₂ fluoroalkoxy, —OCH₂CH═CH₂, —OCH₂C═CH, —NR_{c}R_{c}, or a cyclic group selected from C₃₋₆ cycloalkyl, 3- to 6-membered heterocyclyl, phenyl, monocyclic heteroaryl, and bicyclic heteroaryl, each cyclic group substituted with zero to 3 substituents independently selected from F, Cl, Br, —OH, —CN, C₁₋₄ alkyl, C₁₋₂ fluoroalkyl, C₁₋₃ alkoxy, C₁₋₂ fluoroalkoxy, and —NR_{c}R_{c};
R_{4c} is C₁₋₄ alkyl or C₃₋₆ cycloalkyl, each substituted with zero to 4 substituents independently selected from F, Cl, —OH, C₁₋₂ alkoxy, C₁₋₂ fluoroalkoxy, and —CN;
and each R₅ is independently —CN, C₁₋₅ alkyl substituted with zero to 4 R_{g}, C₂₋₃ alkenyl substituted with zero to 4 R_{g}, C₂₋₃ alkynyl substituted with zero to 4 R_{g}, C₃₋₄ cycloalkyl substituted with zero to 4 R_{g}, phenyl substituted with zero to 3 R_{g}, oxadiazolyl substituted with zero to 3 R_{g}, pyridinyl substituted with zero to 3 R_{g}, —(CH₂)₁₋₂(heterocyclyl substituted with zero to 4 R_{g}), —(CH₂)₁₋₂NR_{c}C(O)(C₁₋₄ alkyl), —(CH₂)₁₋₂NR_{c}C(O)O(C₁₋₄ alkyl), —(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl).

23. The method of claim 22, wherein the inhibitor of DGKα and/or DGKζ is a compound of Formula (I) or a pharmaceutically acceptable salt thereof having the structure: 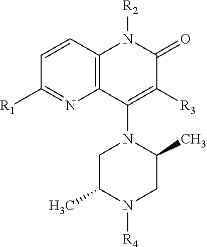
wherein:
R₁ is —CN;
R₂ is —CH₃;
R₃ is H, F, or —CN;
R₄ is:

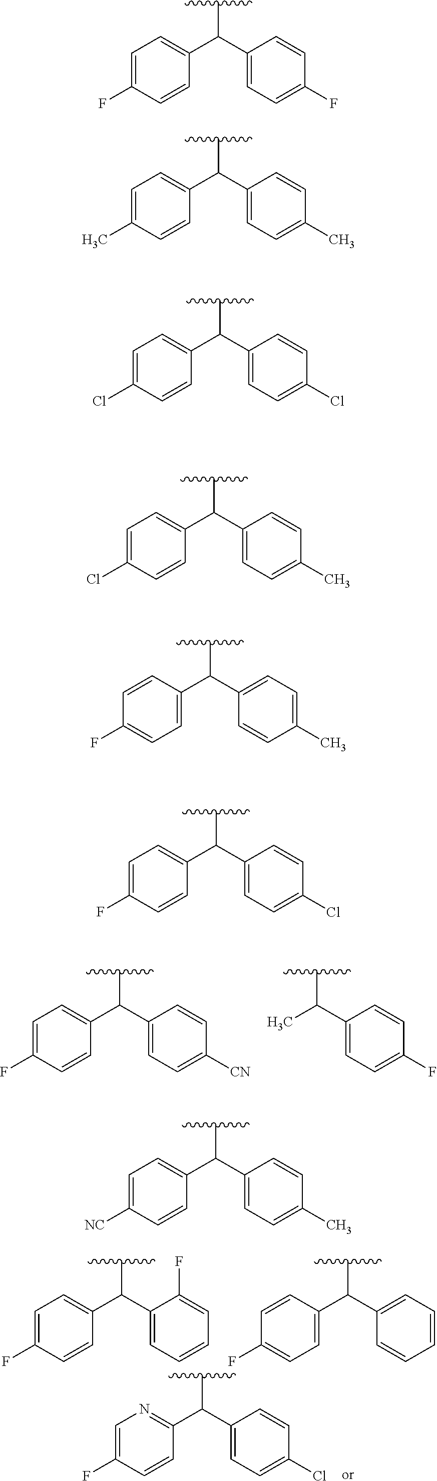

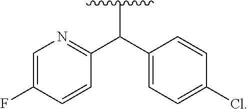 [SMILES: *C(c1ccc(Cl)cc1)c1ccc(F)cn1]

24. The method of claim 21, wherein the inhibitor of DGKα and/or DGKζ is a compound of Formula (I) or a pharmaceutically acceptable salt thereof having the structure:

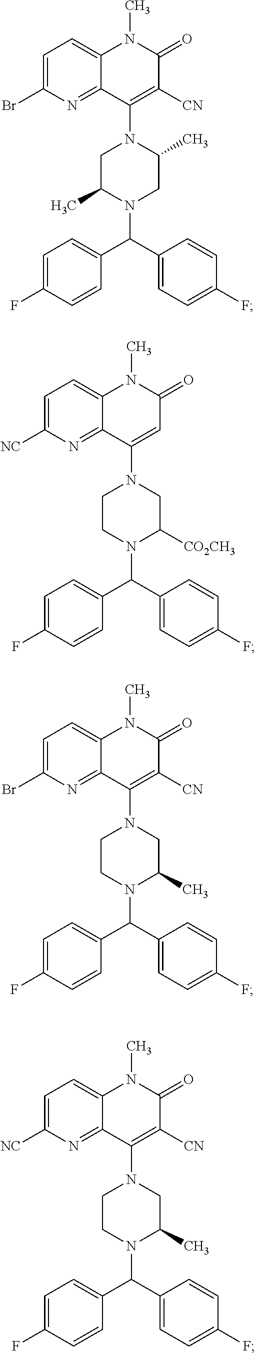 [SMILES: C[C@@H]1CN(C(c2ccc(F)cc2)c2ccc(F)cc2)[C@@H](C)CN1c1c(C#N)c(=O)n(C)c2ccc(Br)nc12 ; COC(=O)C1CN(c2cc(=O)n(C)c3ccc(C#N)nc23)CCN1C(c1ccc(F)cc1)c1ccc(F)cc1 ; C[C@@H]1CN(c2c(C#N)c(=O)n(C)c3ccc(Br)nc23)CCN1C(c1ccc(F)cc1)c1ccc(F)cc1 ; C[C@@H]1CN(c2c(C#N)c(=O)n(C)c3ccc(C#N)nc23)CCN1C(c1ccc(F)cc1)c1ccc(F)cc1]

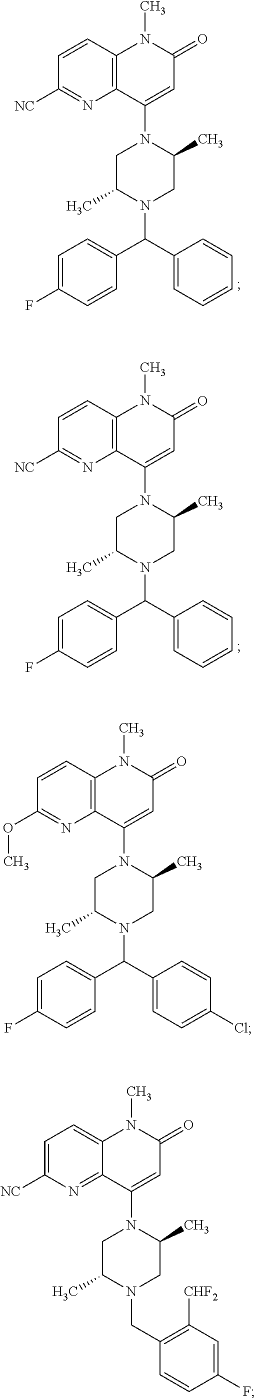 [SMILES: C[C@@H]1CN(c2cc(=O)n(C)c3ccc(C#N)nc23)[C@@H](C)CN1C(c1ccccc1)c1ccc(F)cc1 ; C[C@@H]1CN(c2cc(=O)n(C)c3ccc(C#N)nc23)[C@@H](C)CN1C(c1ccccc1)c1ccc(F)cc1 ; COc1ccc2c(n1)c(N1C[C@@H](C)N(C(c3ccc(F)cc3)c3ccc(Cl)cc3)C[C@@H]1C)cc(=O)n2C ; C[C@@H]1CN(c2cc(=O)n(C)c3ccc(C#N)nc23)[C@@H](C)CN1Cc1ccc(F)cc1C(F)F]

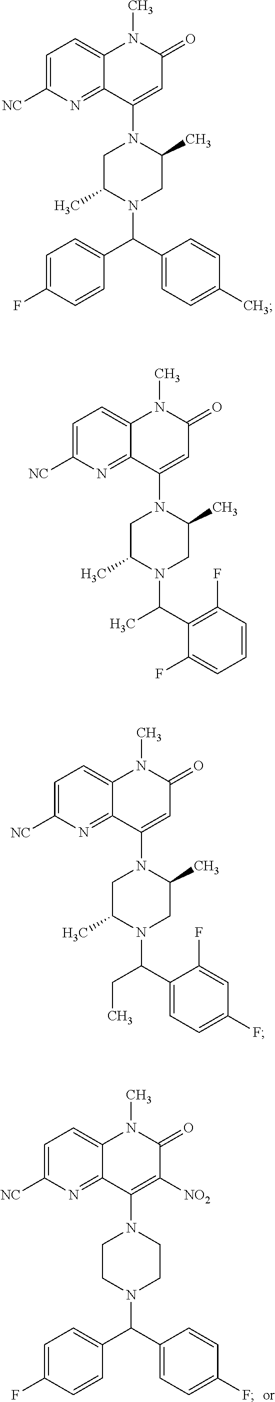 [SMILES: Cc1ccc(C(c2ccc(F)cc2)N2C[C@H](C)N(c3cc(=O)n(C)c4ccc(C#N)nc34)C[C@H]2C)cc1 ; CC(c1c(F)cccc1F)N1C[C@H](C)N(c2cc(=O)n(C)c3ccc(C#N)nc23)C[C@H]1C ; CCC(c1ccc(F)cc1F)N1C[C@H](C)N(c2cc(=O)n(C)c3ccc(C#N)nc23)C[C@H]1C ; Cn1c(=O)c([N+](=O)[O-])c(N2CCN(C(c3ccc(F)cc3)c3ccc(F)cc3)CC2)c2nc(C#N)ccc21]

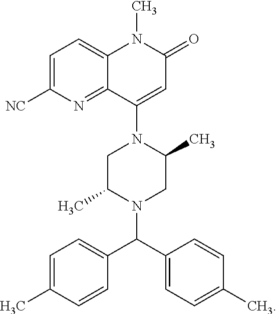 [SMILES: Cc1ccc(C(c2ccc(C)cc2)N2C[C@H](C)N(c3cc(=O)n(C)c4ccc(C#N)nc34)C[C@H]2C)cc1]

25. The method of any one of claims 1-20, wherein the inhibitor of DGKα and/or DGKζ is a compound of Formula (II): 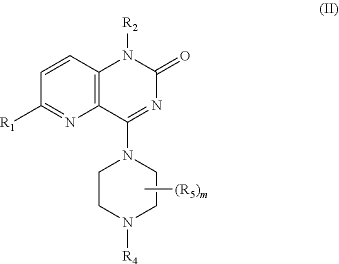
or a salt thereof, wherein:
R₁ is H, F, Cl, Br, —CN, —OH, C₁₋₃ alkyl substituted with zero to 4 R₁ₐ, C₃₋₄ cycloalkyl substituted with zero to 4 R₁ₐ, C₁₋₃ alkoxy substituted with zero to 4 R₁ₐ, —NRₐRₐ, —S(O)ₙRₑ, or —P(O)RₑRₑ;
each R₁ₐ is independently F, Cl, —CN, —OH, —OCH₃, or —NRₐRₐ;
each Rₐ is independently H or C₁₋₃ alkyl;
each Rₑ is independently C₃₋₄ cycloalkyl or C₁₋₃ alkyl substituted with zero to 4 R₁ₐ;
R₂ is H, C₁₋₃ alkyl substituted with zero to 4 R₂ₐ, or C₃₋₄ cycloalkyl substituted with zero to 4 R₂ₐ;
each R₂ₐ is independently F, Cl, —CN, —OH, —O(C₁₋₂ alkyl), C₃₋₄ cycloalkyl, C₃₋₄ alkenyl, or C₃₋₄ alkynyl;
R₄ is —CH₂R₄ₐ, —CH₂CH₂R₄ₐ, —CH₂CHR₄ₐR₄ₐ, —CHR₄ₐR_{4b}, or —CR₄ₐR_{4b}R_{4c};
R₄ₐ and R_{4b} are independently:
(i) —CN or C₁₋₆ alkyl substituted with zero to 4 substituents independently selected from F, Cl, —CN, —OH, —OCH₃, —SCH₃, C₁₋₃ fluoroalkoxy, —NRₐRₐ, —S(O)₂Rₑ, or —NRₐS(O)₂Rₑ;
(ii) C₃₋₆ cycloalkyl, 4- to 10-membered heterocyclyl, phenyl, or 5- to 10-membered heteroaryl, each substituted with zero to 4 substituents independently selected from F, Cl, Br, —CN, —OH, C₁₋₆ alkyl, C₁₋₃ fluoroalkyl, C₁₋₂ bromoalkyl, C₁₋₂ cyanoalkyl, C₁₋₄ hydroxyalkyl, —(CH₂)₁₋₂O(C₁₋₃ alkyl), C₁₋₄ alkoxy, C₁₋₃ fluoroalkoxy, C₁₋₃ cyanoalkoxy, —O(C₁₋₄ hydroxyalkyl), —O(CRₓRₓ)₁₋₃O(C₁₋₃ alkyl), C₁₋₃ fluoroalkoxy, —O(CH₂)₁₋₃NR_{c}R_{c}, —OCH₂CH═CH₂, —OCH₂C═CH, —C(O)(C₁₋₄ alkyl), —C(O)OH, —C(O)O(C₁₋₄ alkyl), —NR_{c}R_{c}, —CH₂NRₐRₐ, —NRₐS(O)₂(C₁₋₃ alkyl), —NRₐC(O)(C₁₋₃ alkyl), —(CRₓRₓ)₀₋₂NRₐC(O)O(C₁₋₄ alkyl), —P(O)(C₁₋₃ alkyl)₂, —S(O)₂(C₁₋₃ alkyl), —(CRₓRₓ)₁₋₂(C₃₋₄ cycloalkyl), —(CRₓRₓ)₁₋₂(morpholinyl), —(CRₓRₓ)₁₋₂(difluoromorpholinyl), —(CRₓRₓ)₁₋₂(dimethylmorpholinyl), —(CRₓRₓ)₁₋₂(oxaazabicyclo[2.2.1]heptanyl), (CRₓRₓ)₁₋₂(oxaazaspiro[3.3]heptanyl), —(CRₓRₓ)₁₋₂(methylpiperazinonyl), —(CRₓRₓ)₁₋₂(acetylpiperazinyl), —(CRₓRₓ)₁₋₂(piperidinyl), —(CRₓRₓ)₁₋₂(difluoropiperidinyl), —(CRₓRₓ)₁₋₂(methoxypiperidinyl), —(CRₓRₓ)₁₋₂(hydroxypiperidinyl), —O(CRₓRₓ)₀₋₂(C₃₋₆ cycloalkyl), —O(CRₓRₓ)₀₋₂(methylcyclopropyl), —O(CRₓRₓ)₀₋₂((ethoxycarbonyl)cyclopropyl), —O(CRₓRₓ)₀₋₂(oxetanyl), —O(CRₓRₓ)₀₋₂(methylazetidinyl), —O(CRₓRₓ)₀₋₂(tetrahydropyranyl), —O(CRₓRₓ)₁₋₂(morpholinyl), —O(CRₓRₓ)₀₋₂(thiazolyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, dioxolanyl, pyrrolidinonyl, and R_{d}; or
(iii) C₁₋₄ alkyl substituted with one cyclic group selected from C₃₋₆ cycloalkyl, 4- to 10-membered heterocyclyl, mono- or bicyclic aryl, or 5- to 10-membered heteroaryl, said cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C═CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₆ cycloalkyl;
or R₄ₐ and R_{4b} together with the carbon atom to which they are attached form a C₃₋₆ cycloalkyl or a 3- to 6-membered heterocyclyl, each substituted with zero to 3 R_{f};
each R_{f} is independently F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C—CH, —NR_{c}R_{c}, or a cyclic group selected from C₃₋₆ cycloalkyl, 3- to 6-membered heterocyclyl, phenyl, monocyclic heteroaryl, and bicyclic heteroaryl, each cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, and —NR_{c}R_{c};
R_{4c} is C₁₋₆ alkyl or C₃₋₆ cycloalkyl, each substituted with zero to 4 substituents independently selected from F, Cl, —OH, C₁₋₂ alkoxy, C₁₋₂ fluoroalkoxy, and —CN;
R_{4d} is —OCH₃;
each R_{c} is independently H or C₁₋₂ alkyl;
R_{d} is phenyl substituted with zero to 1 substituent selected from F, Cl, —CN, —CH₃, and —OCH₃;
each R₅ is independently —CN, C₁₋₆ alkyl substituted with zero to 4 R_{g}, C₂₋₄ alkenyl substituted with zero to 4 R_{g}, C₂₋₄ alkynyl substituted with zero to 4 R_{g}, C₃₋₄ cycloalkyl substituted with zero to 4 R_{g}, phenyl substituted with zero to 4 R_{g}, oxadiazolyl substituted with zero to 3 R_{g}, pyridinyl substituted with zero to 4 R_{g}, —(CH₂)₁₋₂ (4- to 10-membered heterocyclyl substituted with zero to 4 R_{g}), —(CH₂)₁₋₂NR_{c}C(O)(C₁₋₄ alkyl), —(CH₂)₁₋₂NR_{c}C(O)O(C₁₋₄ alkyl), —(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl);
each R_{g} is independently F, Cl, —CN, —OH, C₁₋₃ alkoxy, C₁₋₃ fluoroalkoxy, —O(CH₂)₁₋₂O(C₁₋₂ alkyl), or —NR_{c}R_{c};
m is zero, 1, 2, or 3; and
n is zero, 1, or 2.

26. The method of claim 25, wherein the inhibitor of DGKα and/or DGKζ is a compound of Formula (II) or a pharmaceutically acceptable salt thereof, wherein:
R₁ is H, F, Cl, Br, —CN, —OH, C₁₋₃ alkyl substituted with zero to 4 R₁ₐ, cyclopropyl substituted with zero to 3 R₁ₐ, C₁₋₃ alkoxy substituted with zero to 3 R₁ₐ, —NRₐRₐ, —S(O)ₙCH₃, or —P(O)(CH₃)₂;
R₂ is H or C₁₋₂ alkyl substituted with zero to 2 R₂ₐ;
each R₂ₐ is independently F, Cl, —CN, —OH, —O(C₁₋₂ alkyl), cyclopropyl, C₃₋₄ alkenyl, or C₃₋₄ alkynyl;
R₄ₐ and R_{4b} are independently:
(i) —CN or C₁₋₄ alkyl substituted with zero to 4 substituents independently selected from F, Cl, —CN, —OH, —OCH₃, —SCH₃, C₁₋₃ fluoroalkoxy, and —NRₐRₐ;
(ii) C₃₋₆ cycloalkyl, 4- to 10-membered heterocyclyl, phenyl, or 5- to 10-membered heteroaryl, each substituted with zero to 4 substituents independently selected from F, Cl, Br, —CN, —OH, C₁₋₆ alkyl, C₁₋₃ fluoroalkyl, C₁₋₂ bromoalkyl, C₁₋₂ cyanoalkyl, C₁₋₂ hydroxyalkyl, —CH₂NRₐRₐ, —(CH₂)₁₋₂O(C₁₋₂ alkyl), —(CH₂)₁₋₂NRₓC(O)O(C₁₋₂ alkyl), C₁₋₄ alkoxy, —O(C₁₋₄ hydroxyalkyl), —O(CRₓRₓ)₁₋₂O(C₁₋₂ alkyl), C₁₋₃ fluoroalkoxy, C₁₋₃ cyanoalkoxy, —O(CH₂)₁₋₂NR_{c}R_{c}, —OCH₂CH═CH₂, —OCH₂C═CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₂ alkyl)₂, —S(O)₂(C₁₋₃ alkyl), —(CH₂)₁₋₂(C₃₋₄ cycloalkyl), —CRₓRₓ(morpholinyl), —CRₓRₓ(difluoromorpholinyl), —CRₓRₓ(dimethylmorpholinyl), —CRₓRₓ(oxaazabicyclo[2.2.1]heptanyl), —CRₓRₓ(oxaazaspiro[3.3]heptanyl), —CRₓRₓ(methylpiperazinonyl), —CRₓRₓ(acetylpiperazinyl), —CRₓRₓ(piperidinyl), —CRₓRₓ(difluoropiperidinyl), —CRₓRₓ(methoxypiperidinyl), —CRₓRₓ(hydroxypiperidinyl), —O(CH₂)₀₋₂(C₃₋₄ cycloalkyl), —O(CH₂)₀₋₂(methylcyclopropyl), —O(CH₂)₀₋₂((ethoxycarbonyl)cyclopropyl), —O(CH₂)₀₋₂(oxetanyl), —O(CH₂)₀₋₂(methylazetidinyl), —O(CH₂)₁₋₂(morpholinyl), —O(CH₂)₀₋₂(tetrahydropyranyl), —O(CH₂)₀₋₂(thiazolyl), cyclopropyl, cyanocyclopropyl, methylazetidinyl, acetylazetidinyl, (tert-butoxycarbonyl)azetidinyl, dioxolanyl, pyrrolidinonyl, triazolyl, tetrahydropyranyl, morpholinyl, thiophenyl, methylpiperidinyl, and R_{d}; or
(iii) C₁₋₃ alkyl substituted with one cyclic group selected from C₃₋₆ cycloalkyl, 4- to 10-membered heterocyclyl, mono- or bicyclic aryl, or 5- to 10-membered heteroaryl, said cyclic group substituted with zero to 3 substituents independently selected from F, Cl, Br, —OH, —CN, C₁₋₃ alkyl, C₁₋₂ fluoroalkyl, C₁₋₃ alkoxy, C₁₋₂ fluoroalkoxy, —OCH₂CH═CH₂, —OCH₂C═CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₄ cycloalkyl;
or R₄ₐ and R_{4b} together with the carbon atom to which they are attached, form a C₃-6 cycloalkyl or a 3- to 6-membered heterocyclyl, each substituted with zero to 3 R_{f};
each R_{f} is independently F, Cl, Br, —OH, —CN, C₁₋₄ alkyl, C₁₋₂ fluoroalkyl, C₁₋₃ alkoxy, C₁₋₂ fluoroalkoxy, —OCH₂CH═CH₂, —OCH₂C—CH, —NR_{c}R_{c}, or a cyclic group selected from C₃₋₆ cycloalkyl, 3- to 6-membered heterocyclyl, phenyl, monocyclic heteroaryl, and bicyclic heteroaryl, each cyclic group substituted with zero to 3 substituents independently selected from F, Cl, Br, —OH, —CN, C₁₋₄ alkyl, C₁₋₂ fluoroalkyl, C₁₋₃ alkoxy, C₁₋₂ fluoroalkoxy, and —NR_{c}R_{c};
R_{4c} is C₁₋₄ alkyl or C₃₋₆ cycloalkyl, each substituted with zero to 4 substituents independently selected from F, Cl, —OH, C₁₋₂ alkoxy, C₁₋₂ fluoroalkoxy, and —CN;
each R₅ is independently —CN, C₁₋₅ alkyl substituted with zero to 4 R_{g}, C₂₋₃ alkenyl substituted with zero to 4 R_{g}, C₂₋₃ alkynyl substituted with zero to 4 R_{g}, C₃₋₄ cycloalkyl substituted with zero to 4 R_{g}, phenyl substituted with zero to 3 R_{g}, oxadiazolyl substituted with zero to 3 R_{g}, pyridinyl substituted with zero to 3 R_{g}, —(CH₂)₁₋₂(4- to 10-membered heterocyclyl substituted with zero to 4 R_{g}), —(CH₂)₁₋₂NR_{c}C(O)(C₁₋₄ alkyl), —(CH₂)₁₋₂NR_{c}C(O)O(C₁₋₄ alkyl), —(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl);
each Rₓ is independently H or —CH₃; and
m is 1, 2, or 3.

27. The method of claim 26, wherein the inhibitor of DGKα and/or DGKζ is a compound of Formula (II) or a pharmaceutically acceptable salt thereof having the structure: 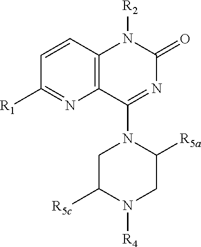
R₁ is —CN;
R₂ is —CH₃;
R₅ₐ is —CH₃ or —CH₂CH₃; and
R_{5c} is —CH₃, —CH₂CH₃, or —CH₂CH₂CH₃.

28. The method of claim 25, wherein the inhibitor of DGKα and/or DGKζ is a compound of Formula (II) or a pharmaceutically acceptable salt thereof having the structure:

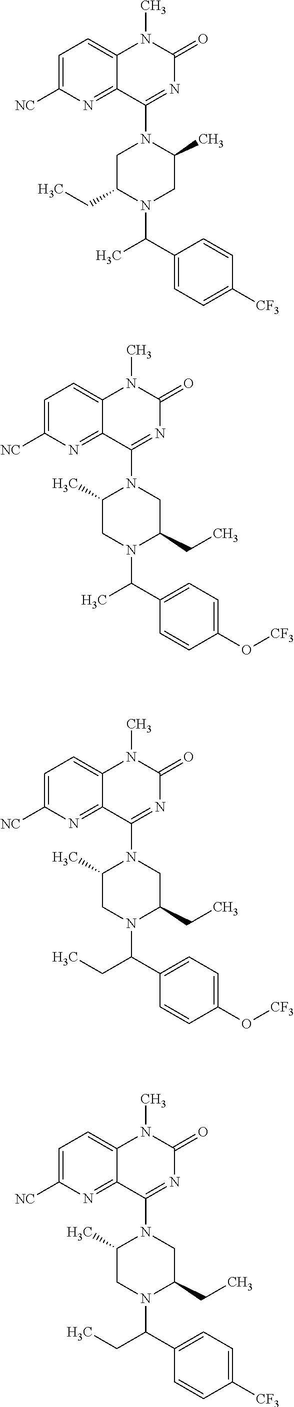 [SMILES: CC[C@@H]1CN(c2nc(=O)n(C)c3ccc(C#N)nc23)[C@@H](C)CN1C(C)c1ccc(C(F)(F)F)cc1 ; CC[C@@H]1CN(c2nc(=O)n(C)c3ccc(C#N)nc23)[C@@H](C)CN1C(C)c1ccc(OC(F)(F)F)cc1 ; CCC(c1ccc(OC(F)(F)F)cc1)N1C[C@H](C)N(c2nc(=O)n(C)c3ccc(C#N)nc23)C[C@H]1CC ; CCC(c1ccc(C(F)(F)F)cc1)N1C[C@H](C)N(c2nc(=O)n(C)c3ccc(C#N)nc23)C[C@H]1CC]

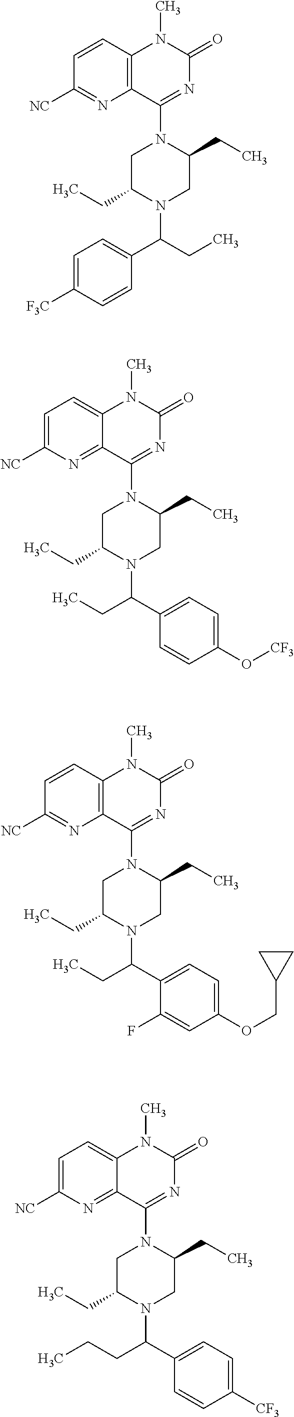 [SMILES: CCC(c1ccc(C(F)(F)F)cc1)N1C[C@H](CC)N(c2nc(=O)n(C)c3ccc(C#N)nc23)C[C@H]1CC ; CCC(c1ccc(OC(F)(F)F)cc1)N1C[C@H](CC)N(c2nc(=O)n(C)c3ccc(C#N)nc23)C[C@H]1CC ; CCC(c1ccc(OCC2CC2)cc1F)N1C[C@H](CC)N(c2nc(=O)n(C)c3ccc(C#N)nc23)C[C@H]1CC ; CCCC(c1ccc(C(F)(F)F)cc1)N1C[C@H](CC)N(c2nc(=O)n(C)c3ccc(C#N)nc23)C[C@H]1CC]

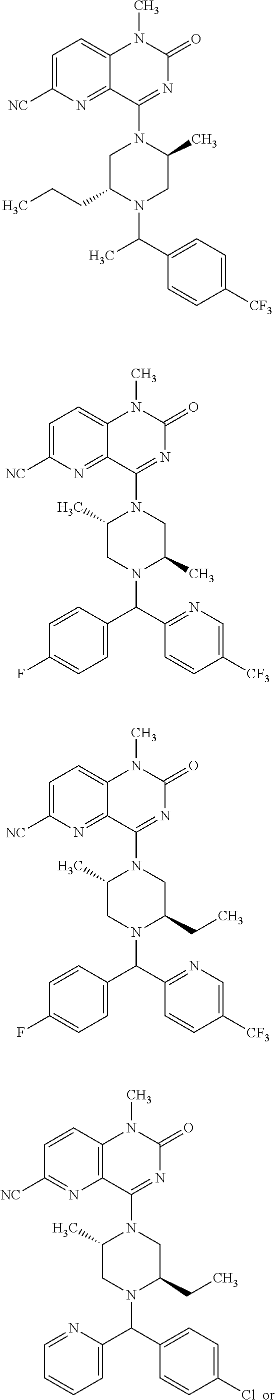 [SMILES: CCC[C@@H]1CN(c2nc(=O)n(C)c3ccc(C#N)nc23)[C@@H](C)CN1C(C)c1ccc(C(F)(F)F)cc1 ; C[C@@H]1CN(c2nc(=O)n(C)c3ccc(C#N)nc23)[C@@H](C)CN1C(c1ccc(F)cc1)c1ccc(C(F)(F)F)cn1 ; CC[C@@H]1CN(c2nc(=O)n(C)c3ccc(C#N)nc23)[C@@H](C)CN1C(c1ccc(F)cc1)c1ccc(C(F)(F)F)cn1 ; CC[C@@H]1CN(c2nc(=O)n(C)c3ccc(C#N)nc23)[C@@H](C)CN1C(c1ccc(Cl)cc1)c1ccccn1]

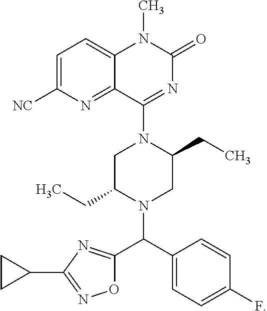 [SMILES: CC[C@H]1CN(C(c2ccc(F)cc2)c2nc(C3CC3)no2)[C@H](CC)CN1c1nc(=O)n(C)c2ccc(C#N)nc12]

29. The method of any one of claims 1-28, wherein the cancer is a solid tumor or a hematological (liquid) tumor.

30. The method of any one of claims 1-29, wherein the cancer is selected from the group of cancers described herein.

31. The method of any one of claims 1-30, wherein the method comprises administering one or more other cancer treatments.

32. The method of claim 31, wherein the one or more other cancer treatments include radiation, surgery, chemotherapy or administration of a biologic drug.

33. The method of claim 31, wherein the one or more other cancer treatments is the administration of a biologic drug and the biologic drug is a drug that stimulates the immune system.

34. The method of any one of claims 1-30, wherein the method does not comprise administering another cancer treatment during the treatment with an inhibitor of DGKα and/or DGKζ, an antagonist of the PD1/PD-L1 axis and/or an antagonist of CTLA4.

35. The method of any one of claims 1-34, wherein the subject has not been treated with an antagonist of the PD1/PD-L1 axis or an antagonist of CTLA4 prior to the administration of an inhibitor of DGKα and/or DGKζ, an antagonist of the PD1/PD-L1 axis and/or an antagonist of CTLA4.

36. The method of claim 35, wherein the method comprises administering to the subject an inhibitor of DGKα and/or DGKζ, an antagonist of the PD1/PD-L1 axis and an antagonist of CTLA4.

37. The method of any one of claims 1-34, wherein the subject is resistant or refractory to treatment with an antagonist of a checkpoint inhibitor, such as an antagonist of the PD1/PD-L1 axis and/or an antagonist of CTLA4.

38. The method of claim 37, wherein the method comprises administering to the subject an inhibitor of DGKα and/or DGKζ, an antagonist of the PD1/PD-L1 axis and an antagonist of CTLA4.

39. The method of claim 21 or claim 25, comprising administering to the subject an antagonist of the PD1/PD-L1 axis and an antagonist of CTLA4.

40. The method of any one of claims 1-39, comprising administering to the subject an antagonist of the PD1/PD-L1 axis and an antagonist of CTLA4, wherein the antagonist of the PD1/PD-L1 axis is a PD1/PD-L1 or CTLA4 antagonist described herein or a variant or derivative thereof.

41. The method of claim 40, wherein the antagonist of the PD1/PD-L1 axis is nivolumab or a variant thereof and the antagonist of CTLA4 is ipilimumab or a variant thereof, e.g., a variant having reduced toxicity relative to ipilimumab.

42. The method of any one of claims 1-3 and 6-41, wherein the inhibitor of DGKα and/or DGKζ is an inhibitor of DGKα and DGKζ.


SEQUENCE LISTING (10 sequences)

SEQ ID NO: 1 - DNA, 2256 bp, Artificial Sequence
  Synthetic Nucleotide sequence encoding hDGK-alpha-(M1-S735)-Ct-TVMV-His
atggccaaggagaggggcctaataagccccagtgattttgcccagctgcaaaaatacatggaatactccaccaaaaaggtcagtgatgtcctaaagctcttcgaggatggcgagatggctaaatatgtccaaggagatgccattgggtacgagggattccagcaattcctgaaaatctatctcgaagtggataatgttcccagacacctaagcctggcactgtttcaatcctttgagactggtcactgcttaaatgagacaaatgtgacaaaagatgtggtgtgtctcaatgatgtttcctgctacttttcccttctggagggtggtcggccagaagacaagttagaattcaccttcaagctgtacgacacggacagaaatgggatcctggacagctcagaagtggacaaaattatcctacagatgatgcgagtggctgaatacctggattgggatgtgtctgagctgaggccgattcttcaggagatgatgaaagagattgactatgatggcagtggctctgtctctcaagctgagtgggtccgggctggggccaccaccgtgccactgctagtgctgctgggtctggagatgactctgaaggacgacggacagcacatgtggaggcccaagaggttccccagaccagtctactgcaatctgtgcgagtcaagcattggtcttggcaaacagggactgagctgtaacctctgtaagtacactgttcacgaccagtgtgccatgaaagccctgccttgtgaagtcagcacctatgccaagtctcggaaggacattggtgtccaatcacatgtgtgggtgcgaggaggctgtgagtccgggcgctgcgaccgctgtcagaaaaagatccggatctaccacagtctgaccgggctgcattgtgtatggtgccacctagagatccacgatgactgcctgcaagcggtgggccatgagtgtgactgtgggctgctccgggatcacatcctgcctccatcttccatctatcccagtgtcctggcctctggaccggatcgtaaaaatagcaaaacaagccagaagaccatggatgatttaaatttgagcacctctgaggctctgcggattgaccctgttcctaacacccacccacttctcgtctttgtcaatcctaagagtggcgggaagcaggggcagagggtgctctggaagttccagtatatattaaaccctcgacaggtgttcaacctcctaaaggatggtcctgagatagggctccgattattcaaggatgttcctgatagccggattttggtgtgtggtggagacggcacagtaggctggattctagagaccattgacaaagctaacttgccagttttgcctcctgttgctgtgttgcccctgggtactggaaatgatctggctcgatgcctaagatggggaggaggttatgaaggacagaatctggcaaagatcctcaaggatttagagatgagtaaagtggtacatatggatcgatggtctgtggaggtgatacctcaacaaactgaagaaaaaagtgacccagtcccctttcaaatcatcaataactacttctctattggcgtggatgcctctattgctcatcgattccacatcatgcgagagaaatatccggagaagttcaacagcagaatgaagaacaagctatggtacttcgaatttgccacatctgaatccatcttctcaacatgcaaaaagctggaggagtctttgacagttgagatctgtgggaaaccgctggatctgagcaacctgtccctagaaggcatcgcagtgctaaacatccctagcatgcatggtggctccaacctctggggtgataccaggagaccccatggggatatctatgggatcaaccaggccttaggtgctacagctaaagtcatcaccgaccctgatatcctgaaaacctgtgtaccagacctaagtgacaagagactggaagtggttgggctggagggtgcaattgagatgggccaaatctataccaagctcaagaatgctggacgtcggctggccaagtgctctgagatcaccttccacaccacaaaaacccttcccatgcaaattgacggagaaccctggatgcagacgccctgtacaatcaagatcacccacaagaaccagatgcccatgctcatgggcccacccccccgctccaccaatttctttggcttcttgagcggatcctcggagacagtgcggtttcagggacaccaccaccatcaccactga

SEQ ID NO: 2 - protein, 751 aa, Artificial Sequence
  Synthetic Amino acid sequence of hDGK-alpha-(M1-S735)-Ct-TVMV-His
MAKERGLISPSDFAQLQKYMEYSTKKVSDVLKLFEDGEMAKYVQGDAIGYEGFQQFLKIYLEVDNVPRHLSLALFQSFETGHCLNETNVTKDVVCLNDVSCYFSLLEGGRPEDKLEFTFKLYDTDRNGILDSSEVDKIILQMMRVAEYLDWDVSELRPILQEMMKEIDYDGSGSVSQAEWVRAGATTVPLLVLLGLEMTLKDDGQHMWRPKRFPRPVYCNLCESSIGLGKQGLSCNLCKYTVHDQCAMKALPCEVSTYAKSRKDIGVQSHVWVRGGCESGRCDRCQKKIRIYHSLTGLHCVWCHLEIHDDCLQAVGHECDCGLLRDHILPPSSIYPSVLASGPDRKNSKTSQKTMDDLNLSTSEALRIDPVPNTHPLLVFVNPKSGGKQGQRVLWKFQYILNPRQVFNLLKDGPEIGLRLFKDVPDSRILVCGGDGTVGWILETIDKANLPVLPPVAVLPLGTGNDLARCLRWGGGYEGQNLAKILKDLEMSKVVHMDRWSVEVIPQQTEEKSDPVPFQIINNYFSIGVDASIAHRFHIMREKYPEKFNSRMKNKLWYFEFATSESIFSTCKKLEESLTVEICGKPLDLSNLSLEGIAVLNIPSMHGGSNLWGDTRRPHGDIYGINQALGATAKVITDPDILKTCVPDLSDKRLEVVGLEGAIEMGQIYTKLKNAGRRLAKCSEITFHTTKTLPMQIDGEPWMQTPCTIKITHKNQMPMLMGPPPRSTNFFGFLSGSSETVRFQGHHHHHH

SEQ ID NO: 3 - DNA, 2838 bp, Artificial Sequence
  Synthetic Nucleotide sequence encoding hDGK-zeta-(M1-A928)-transcript variant-2 Ct-TVMV-His
atggagccgcgggacggtagccccgaggcccggagcagcgactccgagtcggcttccgcctcgtccagcggctccgagcgcgacgccggtcccgagccggacaaggcgccgcggcgactcaacaagcggcgcttcccggggctgcggctcttcgggcacaggaaagccatcacgaagtcgggcctccagcacctggccccccctccgcccacccctggggccccgtgcagcgagtcagagcggcagatccggagtacagtggactggagcgagtcagcgacatatggggagcacatctggttcgagaccaacgtgtccggggacttctgctacgttggggagcagtactgtgtagccaggatgctgcagaagtcagtgtctcgaagaaagtgcgcagcctgcaagattgtggtgcacacgccctgcatcgagcagctggagaagataaatttccgctgtaagccgtccttccgtgaatcaggctccaggaatgtccgcgagccaacctttgtacggcaccactgggtacacagacgacgccaggacggcaagtgtcggcactgtgggaagggattccagcagaagttcaccttccacagcaaggagattgtggccatcagctgctcgtggtgcaagcaggcataccacagcaaggtgtcctgcttcatgctgcagcagatcgaggagccgtgctcgctgggggtccacgcagccgtggtcatcccgcccacctggatcctccgcgcccggaggccccagaatactctgaaagcaagcaagaagaagaagagggcatccttcaagaggaagtccagcaagaaagggcctgaggagggccgctggagacccttcatcatcaggcccaccccctccccgctcatgaagcccctgctggtgtttgtgaaccccaagagtgggggcaaccagggtgcaaagatcatccagtctttcctctggtatctcaatccccgacaagtcttcgacctgagccagggagggcccaaggaggcgctggagatgtaccgcaaagtgcacaacctgcggatcctggcgtgcgggggcgacggcacggtgggctggatcctctccaccctggaccagctacgcctgaagccgccaccccctgttgccatcctgcccctgggtactggcaacgacttggcccgaaccctcaactggggtgggggctacacagatgagcctgtgtccaagatcctctcccacgtggaggaggggaacgtggtacagctggaccgctgggacctccacgctgagcccaaccccgaggcagggcctgaggaccgagatgaaggcgccaccgaccggttgcccctggatgtcttcaacaactacttcagcctgggctttgacgcccacgtcaccctggagttccacgagtctcgagaggccaacccagagaaattcaacagccgctttcggaataagatgttctacgccgggacagctttctctgacttcctgatgggcagctccaaggacctggccaagcacatccgagtggtgtgtgatggaatggacttgactcccaagatccaggacctgaaaccccagtgtgttgttttcctgaacatccccaggtactgtgcgggcaccatgccctggggccaccctggggagcaccacgactttgagccccagcggcatgacgacggctacctcgaggtcattggcttcaccatgacgtcgttggccgcgctgcaggtgggcggacacggcgagcggctgacgcagtgtcgcgaggtggtgctcaccacatccaaggccatcccggtgcaggtggatggcgagccctgcaagcttgcagcctcacgcatccgcatcgccctgcgcaaccaggccaccatggtgcagaaggccaagcggcggagcgccgcccccctgcacagcgaccagcagccggtgccagagcagttgcgcatccaggtgagtcgcgtcagcatgcacgactatgaggccctgcactacgacaaggagcagctcaaggaggcctctgtgccgctgggcactgtggtggtcccaggagacagtgacctagagctctgccgtgcccacattgagagactccagcaggagcccgatggtgctggagccaagtccccgacatgccagaaactgtcccccaagtggtgcttcctggacgccaccactgccagccgcttctacaggatcgaccgagcccaggagcacctcaactatgtgactgagatcgcacaggatgagatttatatcctggaccctgagctgctgggggcatcggcccggcctgacctcccaacccccacttcccctctccccacctcaccctgctcacccacgccccggtcactgcaaggggatgctgcaccccctcaaggtgaagagctgattgaggctgccaagaggaacgacttctgtaagctccaggagctgcaccgagctgggggcgacctcatgcaccgagacgagcagagtcgcacgctcctgcaccacgcagtcagcactggcagcaaggatgtggtccgctacctgctggaccacgcccccccagagatccttgatgcggtggaggaaaacggggagacctgtttgcaccaagcagcggccctgggccagcgcaccatctgccactacatcgtggaggccggggcctcgctcatgaagacagaccagcagggcgacactccccggcagcgggctgagaaggctcaggacaccgagctggccgcctacctggagaaccggcagcactaccagatgatccagcgggaggaccaggagacggctgtgggatcctcggagacagtgcggtttcagggacaccaccaccatcaccactga

SEQ ID NO: 4 - protein, 945 aa, Artificial Sequence
  Synthetic Amino acid sequence of hDGK-zeta- (M1-A928)-transcript variant-2 Ct-TVMV-His
MEPRDGSPEARSSDSESASASSSGSERDAGPEPDKAPRRLNKRRFPGLRLFGHRKAITKSGLQHLAPPPPTPGAPCSESERQIRSTVDWSESATYGEHIWFETNVSGDFCYVGEQYCVARMLQKSVSRRKCAACKIVVHTPCIEQLEKINFRCKPSFRESGSRNVREPTFVRHHWVHRRRQDGKCRHCGKGFQQKFTFHSKEIVAISCSWCKQAYHSKVSCFMLQQIEEPCSLGVHAAVVIPPTWILRARRPQNTLKASKKKKRASFKRKSSKKGPEEGRWRPFIIRPTPSPLMKPLLVFVNPKSGGNQGAKIIQSFLWYLNPRQVFDLSQGGPKEALEMYRKVHNLRILACGGDGTVGWILSTLDQLRLKPPPPVAILPLGTGNDLARTLNWGGGYTDEPVSKILSHVEEGNVVQLDRWDLHAEPNPEAGPEDRDEGATDRLPLDVFNNYFSLGFDAHVTLEFHESREANPEKFNSRFRNKMFYAGTAFSDFLMGSSKDLAKHIRVVCDGMDLTPKIQDLKPQCVVFLNIPRYCAGTMPWGHPGEHHDFEPQRHDDGYLEVIGFTMTSLAALQVGGHGERLTQCREVVLTTSKAIPVQVDGEPCKLAASRIRIALRNQATMVQKAKRRSAAPLHSDQQPVPEQLRIQVSRVSMHDYEALHYDKEQLKEASVPLGTVVVPGDSDLELCRAHIERLQQEPDGAGAKSPTCQKLSPKWCFLDATTASRFYRIDRAQEHLNYVTEIAQDEIYILDPELLGASARPDLPTPTSPLPTSPCSPTPRSLQGDAAPPQGEELIEAAKRNDFCKLQELHRAGGDLMHRDEQSRTLLHHAVSTGSKDVVRYLLDHAPPEILDAVEENGETCLHQAAALGQRTICHYIVEAGASLMKTDQQGDTPRQRAEKAQDTELAAYLENRQHYQMIQREDQETAVGSSETVRFQGHHHHHH

SEQ ID NO: 5 - protein, 8 aa, Artificial Sequence
  Synthetic OVA peptide
SIINFEKL

SEQ ID NO: 6 - protein, 8 aa, Artificial Sequence
  Synthetic A2 peptide
SAINFEKL

SEQ ID NO: 7 - protein, 8 aa, Artificial Sequence
  Synthetic Q4 peptide
SIIQFEKL

SEQ ID NO: 8 - protein, 8 aa, Artificial Sequence
  Synthetic T4 peptide
SIITFEKL

SEQ ID NO: 9 - protein, 8 aa, Artificial Sequence
  Synthetic Q4H7 peptide
SIIQFEHL

SEQ ID NO: 10 - protein, 8 aa, Artificial Sequence
  Synthetic Scrambled peptide
FILKSINE